(19) United States
(12) Patent Application Publication
(10) Pub. No.: US 2024/0344078 A1
(43) Pub. Date: Oct. 17, 2024

(54) GENETICALLY ALTERED NFR5 RECEPTOR KINASES

(71) Applicant: Aarhus Universitet, Aarhus (DK)

(72) Inventors: Elena Simona RADUTOIU, Aarhus (DK); Kasper Røjkjær ANDERSEN, Aarhus (DK); Magdalini TSITSIKLI, Aarhus (DK); Simon Boje HANSEN, Aarhus (DK); Camilla Gottlieb ANDERSEN, Aarhus (DK); Damiano LIRONI, Aarhus (DK); Christina KRÖNAUER, Aarhus (DK); Maria Meisner LARSEN, Aarhus (DK); Kira Dorothea GYSEL, Aarhus (DK); Marie Vogel KOLTE, Aarhus (DK); Jens Stougaard JENSEN, Aarhus (DK); Bine Wissendorf SIMONSEN, Aarhus (DK)

(73) Assignee: Aarhus Universitet, Aarhus (DK)

(21) Appl. No.: 18/605,686

(22) Filed: Mar. 14, 2024

Related U.S. Application Data
(60) Provisional application No. 63/490,195, filed on Mar. 14, 2023.

(51) Int. Cl. C12N 15/82; C07K 14/415; C12N 9/22; C12N 15/11; C12N 15/74

(52) U.S. Cl. CPC C12N 15/8279; C07K 14/415; C12N 9/22; C12N 15/11; C12N 15/743; C12N 15/8201; C12N 15/8227; C12N 2310/20

(57) ABSTRACT

The present disclosure relates to modified plant LysM receptor polypeptides with modified intracellular domains, specifically, modified αA motifs and/or modified αA′ motifs in the juxtamembrane domain. The present disclosure further relates to genetically modified plants including the modified plant LysM receptor polypeptides, and methods of producing the same.

### CROSS REFERENCE TO RELATED APPLICATIONS

This application claims the benefit of U.S. Provisional Application No. 63/490,195, filed Mar. 14, 2023, which is hereby incorporated by reference in its entirety.

### REFERENCE TO AN ELECTRONIC SEQUENCE LISTING

The contents of the electronic sequence listing (7794542002400SEQLIST.xml; Size: 217,902 bytes; and Date of Creation: Mar. 13, 2024) are herein incorporated by reference in their entirety.

### TECHNICAL FIELD

The present disclosure relates to modified plant LysM receptor polypeptides with modified intracellular domains, specifically, modified αA motifs and/or modified αA′ motifs in the juxtamembrane domain. The present disclosure further relates to genetically modified plants including the modified plant LysM receptor polypeptides, and methods of producing the same.

### BACKGROUND

Plants interact with a wealth of microbes and need to distinguish between those that pose a risk and those that offer a potential benefit. To this end, plants use pattern-recognition transmembrane receptor complexes that perceive microbial-derived carbohydrate signals and mount an intracellular response. These microbial-derived signals include conserved cell-wall components such as chitin from pathogenic fungi (Kaku et al. (2006) PNAS 103, 11086-11091; Willmann et al. (2011) PNAS 108, 19824-19829) and Nod factors in the context of nitrogen-fixing symbiosis with bacteria (rhizobia) (Limpens et al. (2003) Science 302, 630-633; Radutoiu et al. (2003) Nature 425, 585-592; Broghammer et al. (2012) PNAS 109, 13859-13864; Bozsoki et al. (2020) Science 369, 663-670; Gysel et al. (2021) PNAS 118, e2111031118).

All plants use LysM receptor kinases (CERKs) to recognize chitin oligomers (CO6-8; chitooligosaccharides) produced by pathogenic fungi and to activate an immune response. Legume plants, on the other hand, have evolved highly similar LysM receptor kinases (NFRs) with increased sensitivity for decorated chitin oligomers (LCOs; lipochitooligosaccharides) produced by nitrogen-fixing soil bacteria. These receptors provide legumes with an ecological advantage as they are able to initiate symbiosis signaling leading to nodule organogenesis, bacterial infection, and symbiotic nitrogen fixation in nutrient-poor soils. Structurally, CERK and NFR receptors contain three tandem LysM domains in their extracellular region followed by a single-pass transmembrane domain, a juxtamembrane domain, and an intracellular kinase domain. The kinase domains phosphorylate specific substrates and initiate distinct signaling pathways: immunity or symbiosis.

Rhizobia-legume root nodule symbiosis requires the plant LysM receptor kinase pair NFR1 and NFR5 for the perception of Nod factors (Radutoiu et al. (2003) Nature 425, 585-592) and the subsequent initiation of the symbiosis pathway. In Lotus japonicus, NFR1-NFR5 dimerization has been demonstrated to be essential for nodule organogenesis (Rübsam et al. 2023. “Nanobody-Driven Signaling Reveals the Core Receptor Complex in Root Nodule Symbiosis.” Science 379 (6629): 272-77). It has been shown that receptor signaling requires the catalytic activity of the NFR1 protein kinase, while the mechanistic role for the catalytically inactive NFR5 pseudokinase is less well understood. Further, the structural domains of the receptors that are responsible for determining the downstream symbiosis pathway have remained unidentified.

There exists a need to understand the mechanistic role for the NFR5 pseudokinase in the initiation of the symbiosis pathway. More broadly, there is a need to identify the structural domains of LysM receptors that determine the downstream symbiosis pathway is initiated instead of the downstream immunity pathway. The identification of these domains will allow the engineering of existing LysM receptor kinases involved in immunity signaling (CERKs) into LysM receptor kinases involved in symbiosis signaling (NFRs). This is a key piece needed to engineer non-legume receptors so that they are able to activate the intracellular pathway leading to root nodule symbiosis.

### BRIEF SUMMARY

The present disclosure provides the crystal structure of the intracellular domain of the NFR5 receptor, including the juxtamembrane domain and the protein kinase domain. Two distinct and structured regions present in the NFR5 juxtamembrane domain are critical for root nodule symbiosis, in particular the αA motif and the αA′ motif. The αA motif and the αA′ motif in NFR5 are required to ensure that symbiosis signaling is initiated. Thus, the present disclosure identifies key residues and regions that are targets for the engineering of non-legume receptors to activate the intracellular pathway leading to root nodule symbiosis.

An aspect of the disclosure includes a modified plant LysM receptor polypeptide including a first juxtamembrane domain including a first αA motif, wherein the first αA motif has been modified as compared to a second αA motif of an unmodified plant LysM receptor polypeptide by insertion, deletion, or substitution of one or more amino acids, two or more amino acids, three or more amino acids, four or more amino acids, five or more amino acids, six or more amino acids, or all seven amino acids, or wherein the first juxtamembrane domain lacks the first αA motif and the second αA motif is inserted into the corresponding position in the first juxtamembrane domain. In a further embodiment of this aspect, the first αA motif, the second αA motif, or both are selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the first αA motif, the second αA motif, or both are selected, include, or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or correspond to amino acids T286-S292 when aligned to SEQ ID NO: 8 (SEQ ID NO: 159). In a further embodiment of this aspect, which may be combined with any of the preceding embodiments, the first αA motif, the second αA motif, or both include the αA motif consensus sequence including X₁X₂X₃X₄LLX₅, where X₁ is T, I, G, or K; X₂ is A, Q, P, or G; X₃ is D or G; X₄ is K or E; X₅ is S, T, or P. In still another embodiment of this aspect, which may be combined with any of the preceding embodiments, the modification includes disruption of the first αA motif, wherein disruption of the αA motif includes removing, replacing, or substituting one or more residues, two or more residues, three or more residues, four or more residues, five or more residues, six or more residues, seven or more residues, seven or more residues, eight or more residues, nine or more residues, or all ten residues within the first αA motif. An additional embodiment of this aspect includes the modified plant LysM receptor polypeptide being a plant NFR5 LysM receptor polypeptide. In a further embodiment of this aspect, the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In yet another embodiment of this aspect, which may be combined with any of the preceding embodiments that have a NFR5 LysM receptor polypeptide, the modified plant NFR5 LysM receptor polypeptide has reduced NFR5 nodulation signaling, as compared to the unmodified plant NFR5 LysM receptor polypeptide. In still another embodiment of this aspect, which may be combined with any of the preceding embodiments that have a NFR5 LysM receptor polypeptide, the modified αA motif is selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the modified αA motif includes or corresponds to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or corresponds to amino acids T286-S292 when aligned to SEQ ID NO: 8 (SEQ ID NO: 159). In yet another embodiment of this aspect, which may be combined with any of the preceding embodiments, the plant LysM receptor polypeptide is an immunity pseudokinase-type receptor.

Some aspects of the disclosure include a modified plant or part thereof including the modified plant LysM receptor polypeptide of any one of the preceding embodiments. In a further embodiment of this aspect, the modified plant LysM receptor polypeptide includes a modified αA motif selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the modified αA motif includes or corresponds to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or corresponds to amino acids T286-S292 when aligned to SEQ ID NO: 8 (SEQ ID NO: 159). In a further embodiment of this aspect, which may be combined with any of the preceding embodiments, the modified αA motif includes the αA motif consensus sequence including X₁X₂X₃X₄LLX₅, where X₁ is T, I, G, or K; X₂ is A, Q, P, or G; X₃ is D or G; X₄ is K or E; X₅ is S, T, or P. In still another embodiment of this aspect, which may be combined with any of the preceding embodiments, the plant part is a leaf, a stem, a root, a root primordia, a flower, a seed, a fruit, a kernel, a grain, a cell, or a portion thereof. In yet a further embodiment of this aspect, which may be combined with any of the preceding embodiments, the plant is selected from the group of cassava, yam, sweet potato, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, pigcon pea, lentil, Bambara groundnut, lupin, pulses, Medicago spp., Lotus spp., forage legumes, indigo, legume trees, or hemp.

Further aspects of the disclosure include methods of making the genetically modified plant or part thereof of any of the preceding embodiments, including introducing a genetic alteration to the plant or part thereof including a nucleic acid sequence encoding a heterologous plant LysM receptor polypeptide including a juxtamembrane domain including a modified αA motif. In a further embodiment of this aspect, the nucleic acid sequence is operably linked to a promoter. In an additional embodiment of this aspect, the promoter is a root specific promoter, an inducible promoter, a constitutive promoter, or a combination thereof. In still another embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR 1 promoter (SEQ ID NO: 124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothioncin promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In a further embodiment of this aspect, the nucleic acid sequence is inserted into the genome of the plant so that the nucleic acid sequence is operably linked to an endogenous promoter. In another embodiment of this aspect, the endogenous promoter is a root specific promoter.

Additional aspects of the disclosure include methods of making the genetically modified plant or part thereof of any of the preceding embodiments, including genetically modifying the plant or part thereof by transforming the plant or part thereof with one or more gene editing components that target an endogenous nuclear genome sequence encoding an endogenous plant LysM receptor polypeptide including a juxtamembrane domain including an αA motif, wherein the αA motif is genetically modified by removing, replacing, or substituting one or more residues, two or more residues, three or more residues, four or more residues, five or more residues, six or more residues, seven or more residues, seven or more residues, eight or more residues, nine or more residues, or all ten residues within the αA motif. In some embodiments of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

In some embodiments, which may be combined with any of the preceding method embodiments, the modified plant LysM receptor polypeptide is a plant NFR5 LysM receptor polypeptide. An additional embodiment of this aspect includes the NFR5 receptor polypeptide being selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In a further embodiment of this aspect, which may be combined with any of the preceding embodiments that has a NFR5 LysM receptor polypeptide, the modified plant NFR5 LysM receptor polypeptide has reduced NFR5 nodulation signaling, as compared to the unmodified plant NFR5 LysM receptor polypeptide.

In some embodiments, which may be combined with any of the preceding method embodiments, the modified plant LysM receptor polypeptide is an immunity pseudokinase-type receptor.

In some embodiments, which may be combined with any of the preceding method embodiments, the αA motif and/or the modified αA motif is selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the αA motif and/or the modified αA motif includes or corresponds to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or corresponds to amino acids T286-S292 when aligned to SEQ ID NO: 8 (SEQ ID NO: 159). In a further embodiment of this aspect, which may be combined with any of the preceding method embodiments, the αA motif and/or the modified αA motif includes the αA motif consensus sequence including X₁X₂X₃X₄LLX₅, where X₁ is T, I, G, or K; X₂ is A, Q, P, or G; X₃ is D or G; X₄ is K or E; X₅ is S, T, or P.

A further aspect of the disclosure includes methods of identifying a plant LysM receptor polypeptide able to initiate a downstream symbiosis pathway, by: (a) providing a polypeptide sequence or a polypeptide model of a plant NFR5 receptor polypeptide juxtamembrane domain or an αA motif portion thereof and a candidate plant LysM receptor polypeptide; and (b) aligning the candidate plant LysM receptor polypeptide to the polypeptide sequence or the polypeptide model. In a further embodiment of this aspect, the plant LysM receptor polypeptide is able to initiate a downstream symbiosis pathway if it aligns with the αA motif of the NFR5 polypeptide juxtamembrane domain or is structurally similar to the αA motif of the NFR5 polypeptide juxtamembrane domain. In a further embodiment of this aspect, which may be combined with any of the preceding embodiments that has a NFR5 LysM receptor polypeptide, the plant NFR5 receptor αA motif includes SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 159.

An additional aspect of the disclosure includes an expression vector, isolated DNA molecule, or recombinant nucleic acid including one or more nucleic acid sequences encoding a heterologous plant LysM receptor polypeptide including a juxtamembrane domain including a modified αA motif, wherein the one or more nucleic acid sequences are operably linked to at least one expression control sequence. In a further embodiment of this aspect, the plant LysM receptor polypeptide is a NFR5 receptor polypeptide. In another embodiment of this aspect, the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In yet another embodiment of this aspect, the plant LysM receptor polypeptide is an immunity pseudokinase-type receptor. In still another embodiment of this aspect, which may be combined with any of the preceding embodiments, the at least one expression control sequence includes a promoter selected from the group of a root specific promoter, a constitutive promoter, or a combination thereof. In a further embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR1 promoter (SEQ ID NO: 124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In an additional embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

A further aspect of the disclosure includes a modified plant LysM receptor polypeptide including a first juxtamembrane domain including a first αA′ motif, wherein the first αA′ motif has been modified as compared to a second αA′ motif of an unmodified plant LysM receptor polypeptide by insertion, deletion, or substitution of one or more amino acids, two or more amino acids, three or more amino acids, four or more amino acids, five or more amino acids, six or more amino acids, all seven amino acids, or wherein the first juxtamembrane domain lacks the first αA′ motif and the second αA′ motif is inserted into the corresponding position in the first juxtamembrane domain. In a further embodiment of this aspect, the first αA′ motif, the second αA′ motif, or both are selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In an additional embodiment of this aspect, the first αA′ motif, the second αA′ motif, or both are selected, include, or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In yet another embodiment of this aspect, which may be combined with any of the preceding embodiments, the first αA′ motif, the second αA′ motif, or both are selected, include, or correspond to X₆VSX₇X₈X₉X₁₀, where X₆ is G or S; X₇ is G, S, E, D, or Q; X₈ is Y or F; X₉ is V, L, or I; X₁₀ is S, G, or D. In still another embodiment of this aspect, which may be combined with any of the preceding embodiments, the modification includes disruption of the first αA′ motif, wherein disruption of the αA′ motif includes removing, replacing, or substituting one or more residues, two or more residues, three or more residues, four or more residues, five or more residues, six or more residues, or all seven residues within the first αA′ motif. An additional embodiment of this aspect includes the modified plant LysM receptor polypeptide being a plant NFR5 LysM receptor polypeptide. In a further embodiment of this aspect, the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In yet another embodiment of this aspect, which may be combined with any of the preceding embodiments that have a NFR5 LysM receptor polypeptide, the modified plant NFR5 LysM receptor polypeptide has reduced NFR5 nodulation signaling, as compared to the unmodified plant NFR5 LysM receptor polypeptide. In still another embodiment of this aspect, which may be combined with any of the preceding embodiments that have a NFR5 LysM receptor polypeptide, the modified αA′ motif is selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In a further embodiment of this aspect, the modified αA motif includes or corresponds to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In yet another embodiment of this aspect, which may be combined with any of the preceding embodiments, the plant LysM receptor polypeptide is an immunity pseudokinase-type receptor.

Some aspects of the disclosure include a modified plant or part thereof including the modified plant LysM receptor polypeptide of any one of the preceding embodiments. In a further embodiment of this aspect, the modified plant LysM receptor polypeptide includes a modified αA′ motif selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In an additional embodiment of this aspect, the modified αA motif includes or corresponds to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In yet another embodiment of this aspect, which may be combined with any of the preceding embodiments, the modified αA′ motif includes the αA′ motif consensus sequence including X₆ VSX₇X₈X₉X₁₀, where X₆ is G or S; X₇ is G, S, E, D, or Q; X₈ is Y or F; X₉ is V, L, or I; X₁₀ is S, G, or D. In still another embodiment of this aspect, which may be combined with any of the preceding embodiments, the plant part is a leaf, a stem, a root, a root primordia, a flower, a seed, a fruit, a kernel, a grain, a cell, or a portion thereof. In yet a further embodiment of this aspect, which may be combined with any of the preceding embodiments, the plant is selected from the group of cassava, yam, sweet potato, corn, cowpca, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, pigeon pea, lentil, Bambara groundnut, lupin, pulses, Medicago spp., Lotus spp., forage legumes, indigo, legume trees, or hemp.

Further aspects of the disclosure include methods of making the genetically modified plant or part thereof of any of the preceding embodiments, including introducing a genetic alteration to the plant or part thereof including a nucleic acid sequence encoding a heterologous plant LysM receptor polypeptide including a juxtamembrane domain including a modified αA′ motif. In a further embodiment of this aspect, the nucleic acid sequence is operably linked to a promoter. In an additional embodiment of this aspect, the promoter is a root specific promoter, an inducible promoter, a constitutive promoter, or a combination thereof. In still another embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR 1 promoter (SEQ ID NO: 124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In a further embodiment of this aspect, the nucleic acid sequence is inserted into the genome of the plant so that the nucleic acid sequence is operably linked to an endogenous promoter. In another embodiment of this aspect, the endogenous promoter is a root specific promoter.

Additional aspects of the disclosure include methods of making the genetically modified plant or part thereof of any of the preceding embodiments, including genetically modifying the plant or part thereof by transforming the plant or part thereof with one or more gene editing components that target an endogenous nuclear genome sequence encoding an endogenous plant LysM receptor polypeptide including a juxtamembrane domain including an αA′ motif, wherein the αA′ motif is genetically modified by removing, replacing, or substituting one or more residues, two or more residues, three or more residues, four or more residues, five or more residues, six or more residues, or all seven residues within the αA′ motif. In some embodiments of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

In some embodiments, which may be combined with any of the preceding method embodiments, the modified plant LysM receptor polypeptide is a plant NFR5 LysM receptor polypeptide. An additional embodiment of this aspect includes the NFR5 receptor polypeptide being selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In a further embodiment of this aspect, which may be combined with any of the preceding embodiments that has a NFR5 LysM receptor polypeptide, the modified plant NFR5 LysM receptor polypeptide has reduced NFR5 nodulation signaling, as compared to the unmodified plant NFR5 LysM receptor polypeptide.

In some embodiments, which may be combined with any of the preceding method embodiments, the modified plant LysM receptor polypeptide is an immunity pseudokinase-type receptor.

In some embodiments, which may be combined with any of the preceding method embodiments, the αA′ motif and/or the modified αA′ motif is selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In an additional embodiment of this aspect, the αA motif and/or the modified αA motif includes or corresponds to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In a further embodiment of this aspect, which may be combined with any of the preceding method embodiments, the αA motif and/or the modified αA motif includes the αA motif consensus sequence including X₆ VSX₇X₈X₉X₁₀, where X₆ is G or S; X₇ is G, S, E, D, or Q; X₈ is Y or F; X₉ is V, L, or I; X₁₀ is S, G, or D.

A further aspect of the disclosure includes methods of identifying a plant LysM receptor polypeptide able to initiate a downstream symbiosis pathway, by: (a) providing a polypeptide sequence or a polypeptide model of a plant NFR5 receptor polypeptide juxtamembrane domain or an αA′ motif portion thereof and a candidate plant LysM receptor polypeptide; and (b) aligning the candidate plant LysM receptor polypeptide to the polypeptide sequence or the polypeptide model. In a further embodiment of this aspect, the plant LysM receptor polypeptide is able to initiate a downstream symbiosis pathway if it aligns with the αA′ motif of the NFR5 polypeptide juxtamembrane domain or is structurally similar to the αA′ motif of the NFR5 polypeptide juxtamembrane domain. In a further embodiment of this aspect, which may be combined with any of the preceding embodiments that has a NFR5 LysM receptor polypeptide, the plant NFR5 receptor αA′ motif includes SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160.

An additional aspect of the disclosure includes an expression vector, isolated DNA molecule, or recombinant nucleic acid including one or more nucleic acid sequences encoding a heterologous plant LysM receptor polypeptide including a juxtamembrane domain including a modified αA′ motif, wherein the one or more nucleic acid sequences are operably linked to at least one expression control sequence. In a further embodiment of this aspect, the plant LysM receptor polypeptide is a NFR5 receptor polypeptide. In another embodiment of this aspect, the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In yet another embodiment of this aspect, the plant LysM receptor polypeptide is an immunity pseudokinase-type receptor. In still another embodiment of this aspect, which may be combined with any of the preceding embodiments, the at least one expression control sequence includes a promoter selected from the group of a root specific promoter, a constitutive promoter, or a combination thereof. In a further embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR1 promoter (SEQ ID NO: 124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In an additional embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

In a further embodiment of the disclosure, which may be combined with any of the preceding embodiments that have a modified plant LysM receptor polypeptide including a modified αA motif, the modified plant LysM receptor polypeptide further includes a modified αA′ motif. In a further embodiment of the disclosure, which may be combined with any of the preceding embodiments that have a modified plant LysM receptor polypeptide including a modified αA′ motif, the modified plant LysM receptor polypeptide further includes a modified αA motif.

Further aspects of the disclosure include a bacterial cell or an Agrobacterium cell including the expression vector, isolated DNA molecule, or recombinant nucleic acid of any one of the preceding embodiments.

Other aspects of the disclosure include a genetically modified plant, plant part, plant cell, or seed including the expression vector, isolated DNA molecule, or recombinant nucleic acid of any one of the preceding embodiments.

Additional aspect of the disclosure include a kit including the expression vector, isolated DNA molecule, or recombinant nucleic acid of any one of the preceding embodiments, the bacterial cell or the Agrobacterium cell of any one the preceding embodiments, or the genetically modified plant, plant part, plant cell, or seed of any one of the preceding embodiments.

Some aspects of the disclosure include methods of reducing NFR5 nodulation signaling including: introducing a genetic alteration via an expression vector, isolated DNA molecule, or recombinant nucleic acid of any one of the preceding embodiments to a cell. A further embodiment of this aspect includes the plant being a plant cell.

A further aspect of the disclosure includes a modified plant NFR5 LysM receptor polypeptide including a first intracellular domain including a first juxtamembrane domain and a first kinase domain, wherein the first intracellular domain was modified by substituting one or more amino acids in the first intracellular domain with the corresponding amino acids from a second intracellular domain including a second juxtamembrane domain and a second kinase domain. In an additional embodiment of this aspect, the first juxtamembrane domain includes a first αA motif, and wherein the second juxtamembrane domain includes a second αA motif. In a further embodiment of this aspect, the first αA motif, the second αA motif, or both are selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the first αA motif, the second αA motif, or both include or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or correspond to amino acids T286-S292 when aligned to SEQ ID NO: 8 (SEQ ID NO: 159). In still another embodiment of this aspect, which may be combined with any of the preceding embodiments, the first intracellular domain is from a first NFR5 LysM receptor polypeptide of a first plant species, and wherein the second intracellular domain is from a second NFR5 LysM receptor polypeptide of a second plant species. In yet another embodiment of this aspect, which may be combined with any of the preceding embodiments, the first plant species is a legume plant species, and wherein the second plant species is a non-legume plant species; or wherein the first plant species is a non-legume plant species, and the second plant species is a legume plant species. In a further embodiment of this aspect, the legume plant species is selected from the group of bean, soybean, pea, chickpea, cowpea, pigcon pea, Bambara groundnut, lentil, pulses, Medicago spp., Lotus spp., forage legumes, indigo, or legume trees. In another embodiment of this aspect, the non-legume plant species is selected from the group of cassava, yam, sweet potato, corn, rice, barley, wheat, Trema spp., apple, pear, plum, apricot, peach, almond, walnut, strawberry, raspberry, blackberry, red currant, black currant, melon, cucumber, pumpkin, squash, grape, or hemp.

A further aspect of the disclosure includes a modified plant NFR5 LysM receptor polypeptide including a first intracellular domain including a first juxtamembrane domain and a first kinase domain, wherein the first intracellular domain was modified by substituting one or more amino acids in the first intracellular domain with the corresponding amino acids from a second intracellular domain including a second juxtamembrane domain and a second kinase domain. In an additional embodiment of this aspect, the first juxtamembrane domain includes a first αA′ motif, and wherein the second juxtamembrane domain includes a second αA′ motif. In a further embodiment of this aspect, the first αA′ motif, the second αA′ motif, or both are selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In yet another embodiment of this aspect, the first αA′ motif, the second αA′ motif, or both include or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In still another embodiment of this aspect, which may be combined with any of the preceding embodiments, the first intracellular domain is from a first NFR5 LysM receptor polypeptide of a first plant species, and wherein the second intracellular domain is from a second NFR5 LysM receptor polypeptide of a second plant species. In yet another embodiment of this aspect, which may be combined with any of the preceding embodiments, the first plant species is a legume plant species, and wherein the second plant species is a non-legume plant species; or wherein the first plant species is a non-legume plant species, and the second plant species is a legume plant species. In a further embodiment of this aspect, the legume plant species is selected from the group of bean, soybean, pea, chickpea, cowpca, pigeon pea, Bambara groundnut, lentil, pulses, Medicago spp., Lotus spp., forage legumes, indigo, or legume trees. In another embodiment of this aspect, the non-legume plant species is selected from the group of cassava, yam, sweet potato, corn, rice, barley, wheat, Trema spp., apple, pear, plum, apricot, peach, almond, walnut, strawberry, raspberry, blackberry, red currant, black currant, melon, cucumber, pumpkin, squash, grape, or hemp.

A further aspect of the disclosure includes a modified plant NFR5 LysM receptor polypeptide including a first intracellular domain including a first juxtamembrane domain and a first kinase domain, wherein the first intracellular domain was modified by substituting one or more amino acids in the first intracellular domain with the corresponding amino acids from a second intracellular domain including a second juxtamembrane domain and a second kinase domain. In an additional embodiment of this aspect, the first juxtamembrane domain includes a first αA and a first αA′ motif, and wherein the second juxtamembrane domain includes a second αA motif and a second αA′ motif. In a further embodiment of this aspect, the first αA motif, the second αA motif, or both are selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the first αA motif, the second αA motif, or both include or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or correspond to amino acids T286-S292 when aligned to SEQ ID NO: 8 (SEQ ID NO: 159). In a further embodiment of this aspect, the first αA′ motif, the second αA′ motif, or both are selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In yet another embodiment of this aspect, the first αA′ motif, the second αA′ motif, or both include or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In still another embodiment of this aspect, which may be combined with any of the preceding embodiments, the first intracellular domain is from a first NFR5 LysM receptor polypeptide of a first plant species, and wherein the second intracellular domain is from a second NFR5 LysM receptor polypeptide of a second plant species. In yet another embodiment of this aspect, which may be combined with any of the preceding embodiments, the first plant species is a legume plant species, and wherein the second plant species is a non-legume plant species; or wherein the first plant species is a non-legume plant species, and the second plant species is a legume plant species. In a further embodiment of this aspect, the legume plant species is selected from the group of bean, soybean, pea, chickpea, cowpea, pigeon pea, Bambara groundnut, lentil, pulses, Medicago spp., Lotus spp., forage legumes, indigo, or legume trees. In another embodiment of this aspect, the non-legume plant species is selected from the group of cassava, yam, sweet potato, corn, rice, barley, wheat, Trema spp., apple, pear, plum, apricot, peach, almond, walnut, strawberry, raspberry, blackberry, red currant, black currant, melon, cucumber, pumpkin, squash, grape, or hemp.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1 shows constructs used. The first row shows a Thioredoxin, 6×His, and Tobacco Etch Virus (TEV) protease site fusion tag N-terminal to a full-length NFR5 intracellular domain construct, comprising the juxtamembrane region, the kinase core, and the C-terminal tail (NFR5 (residues 276-595), SEQ ID NO: 146). The second row shows the VHH Nb200, designed with a non-cleavable C-terminal 6x-His-tag (6×His (SEQ ID NO: 123), shown in blue).

FIGS. 2A-2F show NFR5 intracellular domain purification. FIG. 2A shows the chromatogram of the initial NFR5 (residues: 276-595; SEQ ID NO: 146) purification via nickel affinity chromatography (Ni-AC). The x-axis indicates volume through the column in mL. The left y-axis and the black line indicate the UV280 milli absorbance units (mAu). The right y-axis and the green line indicate the percentage of total Buffer B (% B) added to the column. Stages of the Ni-AC purification are demarcated by vertical blue lines, and are defined above the graph. FIG. 2B shows SDS-PAGE of fractions from the Ni-AC of FIG. 2A. The first lane, “M”, contains a molecular weight ladder, labeled to the left of the gel picture with the respective kDa amount. Lane “Inp” contains a sample of the input. Lane “FT” contains a fraction from the flow through. Lanes labeled “Wash” contain fractions from the Wash section of the purification, as labeled in FIG. 2A. Fraction numbers are indicated above the gel picture. Lanes labeled “Elution” contain fractions from the Elution section of the purification, as labeled in FIG. 2A. Fraction numbers are indicated above the gel picture. The arrow indicates the protein of interest. FIG. 2C shows the chromatogram of the second NFR5 (residues: 276-595; SEQ ID NO: 146) purification via Ni-AC, following TEV digestion. NFR5 was collected from the column flow through and separated from the Ni-NTA (nickel-nitrilotriacetic acid) captured His-tagged TEV protease and cleaved fusion tag. The x-axis indicates volume through the column in mL. The left y-axis and the black line indicate the UV280 milli absorbance units (mAu). The right y-axis and the green line indicate the percentage of total Buffer B added to the column. Stages of the Ni-AC purification are demarcated by vertical blue lines, and are defined above the graph. FIG. 2D shows SDS-PAGE of fractions from the Ni-AC of FIG. 2C. The first lane, “M”, contains a molecular weight ladder, labeled to the left of the gel picture with the respective kDa amount. Lane “-TEV” contains the non-digested sample. Lane “Inp” contains a sample of the TEV-digested input. Lane “FT” contains a sample from the Flow through stage of the purification, as labeled in FIG. 2C. Lane “Elu” contains a fraction from the Elution section of the purification, as labeled in FIG. 2C. The arrow indicates the protein of interest. FIG. 2E shows the chromatogram of the NFR5 size exclusion chromatography (SEC) purification using a HiLoad® 16/600 Superdex® 75 pg column. The x-axis indicates volume through the column in mL. The y-axis and the black line indicate the UV280 milli absorbance units (mAu). Fractions 16-26 are indicated by vertical blue dotted lines, and were pooled. FIG. 2F shows SDS-PAGE of fractions from the Ni-AC of FIG. 2E. The NFR5 band fits the expected molecular size of ˜36 kDa. The first lane, “M”, contains a molecular weight ladder, labeled to the left of the gel picture with the respective kDa amount. Lane “Inp” contains a sample of the input. Numbered lanes contain samples of the corresponding SEC fractions from FIG. 2E. The arrow indicates the protein of interest.

FIGS. 3A-3B show the functionality of the NFR5 intracellular domain in signaling. FIG. 3A shows SDS-PAGE of a phosphorylation assay of NFR1 (left lane) and NFR5 (right lane). The top image shows staining with Coomassie® brilliant blue, with a molecular weight ladder labeled to the left. The bottom image shows an autoradiograph of the gel from the top image. A band is visible for NFR1, but no band is visible for NFR5. FIG. 3B shows a nodulation assay for complementation of nfr5. A box-and-whisker plot shows the number of nodules per plant on the y-axis formed on nfr5 plants transformed with the construct indicated on the x-axis and infected with M. loti R7A. Cartoon protein schematics of the constructs are shown below the plot. From left to right, the constructs are tYFP, full length NFR5, and NFR5 with a deletion of the kinase domain (“NFR5 Akinase”). Numbers below each plot indicate the number of nodulating plants out of total plants assayed. Different letters above the plot indicate significant differences.

FIGS. 4A-4F show that a VHH binds and forms a stable complex with NFR5, but does not interfere with NFR5 signaling. FIG. 4A shows SDS PAGE analysis of Avi-tagged NFR5. The first lane, “M”, contains a molecular weight ladder, labeled to the left of the gel picture with the respective kDa amount. The second lane, “NFR5 Avitag”, contains the Avi-tagged NFR5 protein preparation (SEQ ID NO: 150). FIG. 4B shows SDS PAGE analysis of VHH Nb200 and VHH Nb200 mutant (A51E Y60E V100E; SEQ ID NO: 151). The first lane, “M”, contains a molecular weight ladder, labeled to the left of the gel picture with the respective kDa amount. The second lane, “Nb200”, contains the VHH Nb200 protein preparation. The third lane, “Nb200 control”, contains the negative control VHH Nb200 mutant (A51E Y60E V100E; SEQ ID NO: 151) protein preparation. FIG. 4C shows the results of a bio-layer interferometry (BLI) assay with Avi-tagged NFR5 assayed against VHH Nb200 at 5 μM, 2.5 μM, 1.3 μM, 0.63 μM, 0.31 μM, 0.16 μM, and 0.08 μM concentration. Data was fitted using an association/dissociation 1:1 binding model with R²=0.99, giving a derived K_{d}=174.4±0.8 nM. The x-axis indicates time in seconds. The y-axis indicates the wavelength shift in nm. FIG. 4D shows the results of a bio-layer interferometry (BLI) assay with Avi-tagged NFR5 assayed against VHH Nb200 control (A51E Y60E V100E; SEQ ID NO: 151) at 5 μM, 2.5 μM, 1.3 μM, 0.63 μM, 0.31 μM, 0.16 μM, and 0.08 μM concentration. The x-axis indicates time in seconds. The y-axis indicates the wavelength shift in nm. FIG. 4E shows a chromatogram of the isolation of the crystallization complex between NFR5 (residues: 276-563; SEQ ID NO: 147) and the VHH Nb200 using size exclusion chromatography (SEC) and an excess of Nb200. Blue dotted lines demarcate fractions with complex formation, labelled as P1. Elution of excess Nb200 is labelled as P2. The x-axis indicates volume through the column in mL. The y-axis indicates the UV₂₈₀ milli absorbance units (mAu). FIG. 4F shows SDS PAGE of the fractions identified in FIG. 4E. The first lane, “M”, contains a molecular weight ladder, labeled to the left of the gel picture with the respective kDa amount. Lane “Inp” contains a sample of the input. Lanes “P1” contain fractions from the P1 complex section of the purification. Lanes “P2” contain fractions from the P2 section of the purification with excess Nb200. The black horizontal line over lanes indicates fractions pooled. Arrows indicate the proteins of interest.

FIGS. 5A-5H show structural determination and analysis of NFR5 intracellular domain (residues: 276-563; SEQ ID NO: 147). FIG. 5A shows an X-ray diffraction image of the NFR5:Nb200 complex. Protein diffraction spots are clearly visible and well resolved (white on black). Data processing detected signal to 2.6 Å. Resolution shells for 5 Å and 2.6 Å are indicated by red circles. The inset in the bottom right corner shows the corresponding crystal. The scale bar in the inset indicates 200 μm. FIG. 5B shows a structure model of NFR5-Nb200. NFR5 is shown in purple on the left. The VHH Nb200 is shown in grey on the bottom right. N-lobe and C-lobe are labelled at the left, and C-lobe α-helices of NFR5 are labelled with white text. FIG. 5C shows a structural model of the NFR5 intracellular domain. Secondary structure clements (α-helices and β-sheets) are indicated with black and white text. The juxtamembrane αA and αA′ helices precede the pseudokinase domain with the N- and C-lobes marked. An ATP molecule from the structure of PKA (PDB: 1ATP, (Zheng et al. (1993), Biochemistry 32, 2154-2161) (labelled “ATP (docked)”) can be docked into the ATP binding pocket of NFR5 without any steric clashes. The K339-E349 salt bridge, diverged DFG to ‘NFA’ motif, and HRD motifs are additionally highlighted. FIG. 5D shows a structural model of the NFR5 intracellular domain, with the juxtamembrane spanning region marked along with the truncated glycine-rich and activation loops. The catalytic spine (C-spine) and regulatory spine (R-spine) are highlighted in yellow and pink transparent surface representations, respectively, showing the C-spine is broken due to the unoccupied ATP pocket, while the R-spine is fully formed. Residues A337, L440 in the C-spine and L353 in the R-spine are highlighted. FIG. 5E shows a model of the NFR5-Nb200 complex resolved in the crystal structure, with NFR5 shown in purple and the VHH Nb200 shown in grey. Secondary structural features (α-helices and β-sheets) are labelled with white text. The right image is a zoom in on the dashed area on the left, showing the major interaction area between Nb200 and NFR5. Nb200 residues A51, Y60, and V100 are shown. NFR5 W501 and W497 are primarily contacted by Nb200. FIG. 5F shows a nodulation assay in wild-type Lotus japonicus (ecotype Gifu). A box-and-whisker plot shows the number of nodules per plant on the y-axis formed on wild-type plants transformed with the construct indicated on the x-axis and infected with M. loti R7A. From left to right, the constructs are tYFP (negative control), overexpression construct of the VHH Nb200 driven by p35S, and overexpression construct of mutant VHH Nb200 driven by p35S. Numbers below each plot indicate the number of nodulating plants out of total plants assayed. Letters above the plot indicate a lack of significant differences. FIG. 5G shows nodulation assays in wild-type Lotus japonicus (ecotype Gifu; left) and Lotus japonicus nfr/mutant plants. A box-and-whisker plot shows the number of nodules per plant on the y-axis formed on the plants indicated at the top transformed with the construct indicated on the x-axis. Cartoon protein schematics of the constructs are shown below the plot. From left to right, the constructs are tYFP, full length NFR1 (white), and fusion of the VHH Nb200 (grey) as a C-terminal tag on the NFR1 receptor (white), which interacts with endogenous NFR5 (purple). Numbers below each plot indicate the number of nodulating plants out of total plants assayed. Different letters above the plot indicate significant differences. FIG. 5H shows nodulation assays in Lotus japonicus nfr5 mutant plants. A box-and-whisker plot shows the number of nodules per plant on the y-axis formed on wild-type plants transformed with the construct indicated on the x-axis and infected with M. loti R7A. Numbers below cach plot indicate the number nodulating plants out of total plants assayed. Different letters above the plot indicate significant differences. Lotus japonicus nfr5 knockout plants are incapable of nodulation when inoculated with M. loti (see first construct, negative control triple YFP transformation marker). Nodulation is restored when complemented with wild-type NFR5 (second column) and many NFR5 mutants targeting classical protein kinase motifs and structural segments. From left to right, constructs are tYFP, NFR5, NFR5 ΔC-term residue 1-563 (c-terminal tail truncated version of NFR5), NFR5 S292A S295A, NFR5 S292D S295D, NFR5 K339A, NFR5 K339 E, NFR5 D433A, NFR5 D443N, NFR5 L440F, NFR5 A337F, and NFR5 L353D. NFR5 S292 S295 are NFR1 phosphorylation sites located in the NFR5 juxtamembrane. NFR5 K339 is a β3 lysine that forms the hallmark electrostatic interaction to αC E349. NFR5 D433 is a catalytic aspartate in the HRD motif. NFR5 L440F and A337F introduce steric occlusion of the ATP binding pocket by mutagenesis of catalytic spine residues. NFR5 L353D disrupts hydrophobic interactions in the regulatory spine.

FIGS. 6A-6J show structural modeling and analysis of the NFR5 intracellular domain. FIG. 6A shows a structural model of the NFR: Nb200 asymmetrical unit (ASU), consisting of two copies of the NFR5-Nb200 complex related by a ˜50° translational rotation. The core protein kinase domain was modelled for both NFR5 chains. An additional large part of the juxtamembrane region was modelled in chain A (purple). Both Nb200 polypeptides (grey) were modelled almost completely, except for a few N- and C-terminal residues and short flexible loops. The two NFR5 molecules contact each other through non-covalent interactions both in the N-lobe and C-lobe. The chain A juxtamembrane segment contacts the ASU chain B C-lobe (bluc). FIG. 6B shows a cartoon partial representation of NFR5 with the juxtamembrane highlighted (full saturation, on the right). Secondary structure elements (α-helices and β-sheets) are indicated in black text. FIG. 6C shows a higher magnification of the juxtamembrane region showing the 2Fo-Fc electron density map (contour σ=1, mesh representation) shown as a grey mesh surrounding the atomic model (cartoon representation). The polypeptide backbone and residue sidechains are well defined in the electron density and support modelling of two α-helices. FIG. 6D shows an electron density (2Fo/Fc) map of the chain A N-lobe, shown as a grey mesh surrounding a cartoon representation with side-chain sticks. The map was contoured at σ=1.0. FIG. 6E shows an electron density (2Fo/Fc) map of the chain B N-lobe, shown as a grey mesh surrounding a cartoon representation with side-chain sticks. The map was contoured at σ=1.0, and is less well-defined than the map of the chain A N-lobe in FIG. 6D; the entire pseudokinase can be modelled, but not the juxtamembrane domain. FIG. 6F shows an overview of the αA and αA′ helices, labelled with white text. The αA residues L290 and L291 form the N-terminal part of the juxtamembrane motif, while the αA′ residues V294, Y297, and V298 form the C-terminal part. The helices are separated by a small linker region imposing a ˜65° bend. S292 and S295 are positioned at the linker bend separating the αA and αA′ helices and have previously been identified as NFR1 phosphorylation sites (Madsen et al. (2011), Plant J 65, 404-417). FIG. 6G shows an electrostatic potential surface representation of the αA and αA′ helices, displaying the hydrophobicity and spatial continuity of the motif. Red indicates more negative, while blue indicates more positive. The juxtamembrane motif is encircled by a red dotted linc. FIG. 6H shows a sequence alignment of Lotus japonicus NFR5 (SEQ ID NO: 125) and barley RLK10 (SEQ ID NO: 126) αA and αA′ along with the sequence logo (SEQ ID NO: 127) of the larger alignment shown in FIG. 6J. The juxtamembrane motif is highlighted in the sequence logo with red boxes below. FIG. 6I shows a conservation surface of the αA and αA′ helices showing the juxtamembrane motif region is conserved throughout NFR5-type receptors. Regions of low conservation are indicated in teal, while regions of high conservation are indicated in purple. The juxtamembrane motif is encircled by a red dotted linc. FIG. 6J shows an amino acid alignment of sequences spanning a first alpha helix (aA), a SG di-peptide linker, and a second alpha helix (aA′) to αB regions in NFR5-type receptors. The aA motif is the last two-thirds of the aA alpha helix and the S of the linker and the aA′ motif is the G of the linker and the entire aA′ alpha helix. The NFR5-type receptors shown are Lotus japonicus (LjNFR5=SEQ ID NO: 128), Medicago truncatula (MINFP=SEQ ID NO: 129), Pea SYM10 (SEQ ID NO: 130; from the full-length sequence of uniprot: Q70KR3, SEQ ID NO: 154), Bean NFR5 (SEQ ID NO: 131; from the full-length sequence of uniprot: V7CHW9, SEQ ID NO: 13), Soybean NFR5A (SEQ ID NO: 132; from the full-length sequence of uniprot: A5YJV9, SEQ ID NO: 158), Chickpea NFP (SEQ ID NO: 133; from the full-length sequence of uniprot: A0A1S3EF43, SEQ ID NO: 6), Lupin NFR5 (SEQ ID NO: 134; from the full-length sequence of uniprot: A0A1J7GXG0, SEQ ID NO: 7), Peanut NFR5 (SEQ ID NO: 135; from the full-length sequence of NCBI: XP_025698788.1, SEQ ID NO: 1), Fragaria NFR5 (SEQ ID NO: 136, from the full-length sequence of NCBI: XP_004300586, SEQ ID NO: 22), Apple NFP (SEQ ID NO: 137; from the full-length sequence of uniprot: A0A498KGG5, SEQ ID NO: 155), Poplar NFR5 (SEQ ID NO: 138; from the full-length sequence of uniprot: A0A2K2AG15, SEQ ID NO: 156), Parasponia NFP2 (SEQ ID NO: 139; from the full-length sequence of Genbank: PON37437.1, SEQ ID NO: 20), Datisca NFR5 (SEQ ID NO: 140; gene: Datgl376S09111), Rice MYR1 (SEQ ID NO: 141; gene: Os03g13080), Barley RLK10 (SEQ ID NO: 142; from the full-length sequence of NCBI: XP_044979240.1, SEQ ID NO: 23), and Maize NFP (SEQ ID: 143; from the full-length sequence of NCBI: XP_020399958.1, SEQ ID NO: 157). The juxtamembrane region upstream and initiating the αA is not conserved. The region surrounding the two exposed hydrophobic surface in αA and αA′ show a high level of specific residue conservation (αA, L290 L291) or high level of conservation in hydrophobicity (αA′, V294 Y297 V298). The conserved Lotus japonicus NFR5 K300 and P301 positions also show high conservation and demarcate the transition from the juxtamembrane to the pseudokinase core. β0, and αB are the first secondary structural elements of the pseudokinase core. A consensus sequence (SEQ ID NO: 145), conservation of each amino acid, and a sequence logo (SEQ ID NO: 144) are displayed below the alignment.

FIGS. 7A-7J show analysis of the functionality of the juxtamembrane motif. FIG. 7A shows an illustration of the NFR5 intracellular domain (left) along with the αA 2E (L290E L291E; SEQ ID NO: 148) (middle) and αA′ 3E (V294E Y297E V298E; SEQ ID NO: 149) mutants (right). Mutations in the a helices are indicated by yellow bars in the cylinders indicating the αA and αA′ helices. FIG. 7B shows an SDS-PAGE gel of purified proteins on the left, and a native PAGE gel on the right. In the SDS-PAGE gel, the first lane, “M”, contains a molecular weight ladder, labeled to the left of the gel picture with the respective kDa amount; Purified NFR5 WT (“WT”, second lanc), NFR5 L290E L291E (“2E” (SEQ ID NO: 148), third lanc), and NFR5 V294E Y297E V298E (“3E” (SEQ ID NO: 149), fourth lanc) appear as homogeneous single bands, each migrating to their respective theoretical molecular sizes. In the native PAGE gel of the purified proteins on the right, the first lane contains bovine serum albumin (BSA) as a positive control; Purified NFR5 WT (“WT”, second lanc), NFR5 L290E L291E (“2E” (SEQ ID NO: 148), third lane), and NFR5 V294E Y297E V298E (“3E” (SEQ ID NO: 149), fourth lane) are not homogeneous in solution and appear with distinct ladder patterns. Regions in which oligomers and monomers are expected are labelled to the right. FIG. 7C shows a graph of Dynamic light scattering (DLS) assay of NFR5 WT revealing a polydispersity index (PDI)>0.2 and particle radii larger than the ˜3.3 nm monomer form, indicating oligomer populations form in solution. The x-axis indicates particle radii in nm. The y-axis indicates the relative frequency in percentages. FIG. 7D shows a graph of Dynamic light scattering (DLS) assay over a concentration series (1-6 mg/ml) of NFR5 WT, showing negative self-interaction parameters (k_{St}=−14.6 mL/g) and self-interaction. The x-axis indicates protein concentration in mg/mL. The y-axis indicates Diffusion coefficient in μm²/s. FIG. 7E shows a graph of Dynamic Light Scattering (DLS) assay over a concentration series (1-6 mg/ml) of NFR5 2E (SEQ ID NO: 148) mutant protein, showing negative self-interaction parameters (k_{St}=−10.8 mL/g) and self-interaction. The x-axis indicates protein concentration in mg/mL. The y-axis indicates Diffusion coefficient in μm²/s. FIG. 7F shows a graph of Dynamic Light Scattering (DLS) assay over a concentration series (1-6 mg/ml) of NFR5 3E (SEQ ID NO: 149) mutant protein, showing negative self-interaction parameters (k_{St}=−7.1 mL/g) and self-interaction. The x-axis indicates protein concentration in mg/mL. The y-axis indicates Diffusion coefficient in μm²/s. FIG. 7G shows an illustration of a bio-layer interferometry interaction experiment. An Avi-tagged version of NFR5 WT (purple) was purified and immobilized on streptavidin biosensors (top box with four-pointed stars representing streptavidin). NFR5 loaded biosensors were assayed against NFR5 WT, 2E (SEQ ID NO: 148), and 3E (SEQ ID NO: 149). FIG. 7H shows a graph of a bio-layer interferometry interaction experiment for interaction between immobilized NFR5 WT and a concentration series of NFR5 WT (0.07-3.6 mg/ml). A clear concentration dependent interaction to NFR5 WT was measured. The x-axis indicates time in seconds; the y-axis indicates nm. FIG. 7I shows a graph of a bio-layer interferometry interaction experiment for interaction between immobilized NFR5 WT and a concentration series of NFR5 2E (SEQ ID NO: 148) mutants (0.07-3.6 mg/ml). No interaction was observed. The x-axis indicates time in seconds; the y-axis indicates nm. FIG. 7J shows a graph of a bio-layer interferometry interaction experiment for interaction between immobilized NFR5 WT and a concentration series of NFR5 3E (SEQ ID NO: 149) mutants (0.07-3.6 mg/ml). No interaction was observed. The x-axis indicates time in seconds; the y-axis indicates nm.

FIGS. 8A-8F show in planta testing of NFR5 and the NFR5 juxtamembrane functionality. FIG. 8A shows an illustration of the Lotus japonicus NFR5 receptor (purple, left) and the NFR5-RLK10 chimeric receptor (right; NFR5 in purple, RLK10 in teal). The chimeric receptor has the LysM ectodomain and ™ helix of NFR5, and the αA helix, αA′ helix, and kinase domain of RLK10. FIG. 8B shows a nodulation assay in Lotus japonicus nfr5 mutants. A box-and-whisker plot shows the number of nodules per plant on the y-axis formed on nfr5 mutant plants transformed with the construct indicated on the x-axis and infected with M. loti R7A. Cartoon protein schematics for each construct (except tYFP) are shown below the plot. From left to right, the constructs are tYFP (negative control), NFR5, NFR5 L290E L291E (“2E”; SEQ ID NO: 148), NFR5 L290F L291F, NFR5 V294E Y297E V298E (“3E”; SEQ ID NO: 149), NFR5/RLK10 chimera, RLK10 L322E L323E (“RLK10 2E”), and RLK10 V326E F329E 1330E (“RLK10 3E”). Numbers below each plot indicate the number of plants assayed. Different letters above the plot indicate significant differences. FIG. 8C shows images of Agrobacterium rhizogenes induced hairy roots for brightfield (top row), YFP (middle row), and DsRED rhizobia marker (bottom row) for Lotus japonicus nfr5 mutant plants inoculated with M. loti and expressing the construct indicated above each column. From left to right, the constructs are pNfr5_NFR5; pNfr5_NFR5 L290E L291E; pNfr5_NFR5 V294E Y297E V298E; pNfr5_NFR5/RLK10 chimera; pNfr5_NFR5/RLK10 L322E L323E; pNfr5_NFR5/RLK10 V326E F329E 1330E; and tYFP marker. Nodules are only visible in nfr5 plants expressing pNfr5_NFR5 or pNfr5_NFR5/RLK10. Scale bar=1 cm. FIG. 8D shows expression and localization of pUbi_NFR5-GFP constructs in Lotus japonicus root protoplasts. From left to right, constructs are: pUbi_NFR5-GFP; pUbi_NFR5-GFP L290E L291E; pUbi_NFR5/RLK10-GFP; and pUbi_NFR5/RLK10-GFP L322E L323E. FIG. 8E shows a nodulation assay in Lotus japonicus nfr5-1 pNIN:GUS mutant plants. A box-and-whisker plot shows the number of nodules per plant on the y-axis formed on the indicated plants transformed with the construct indicated on the x-axis. From left to right, the constructs are empty vector control, wildtype NFR5, and NFR5 Y2971. Numbers below each plot indicate the number of nodulating plants out of the total number of plants assayed. FIG. 8F shows a model of NFR5-NFR5 complex signaling. NFR5 is shown in purple; the cell membrane is shown as a transparent brown box; mutations are shown by yellow stripes. Complex formation is in part mediated by the juxtamembrane motif spanning the αA and αA′ helices. Disruption of this complex, by targeting the juxtamembrane motif, leads to NFR5 receptors incapable of signaling.

FIGS. 9A-9F show an amino acid sequence alignment of NFR5-type LysM receptor sequences from Arachis hypogaea (XP_025698788.1=SEQ ID NO: 1), Arachis ipaensis (XP_016200640.1=SEQ ID NO: 2), Medicago truncatula (Medtr; Medtr8g078300.1=SEQ ID NO: 3; Medtr5g019040.1=SEQ ID NO: 4), Cicer arietinum (XP_004509233.1=SEQ ID NO: 5; XP_012574460.1=SEQ ID NO: 6), Lupinus angustifolius (XP_019420412.1=SEQ ID NO: 7), Lotus japonicus (AER51027.1 (NFR5)=SEQ ID NO: 8; BAI79275.1 (LYS11)=SEQ ID NO: 9), Abrus precatorius (XP_027347386.1=SEQ ID NO: 10; XP_027362672.1=SEQ ID NO: 11), Cajanus cajan (KYP66704.1=SEQ ID NO: 12), and Phaseolus vulgaris (XP_007156886.1=SEQ ID NO: 13). FIG. 9A shows the first portion of the alignment. FIG. 9B shows the second portion of the alignment. FIG. 9C shows the third portion of the alignment. FIG. 9D shows the fourth portion of the alignment. FIG. 9E shows the fifth portion of the alignment. FIG. 9F shows the sixth portion of the alignment.

### DETAILED DESCRIPTION OF THE INVENTION

The following description sets forth exemplary methods, parameters, and the like. It should be recognized, however, that such description is not intended as a limitation on the scope of the present disclosure but is instead provided as a description of exemplary embodiments.

### Modified Plant LysM Receptor Polypeptides and Related Methods

An aspect of the disclosure includes a modified plant LysM receptor polypeptide including a first juxtamembrane domain including a first αA motif, wherein the first αA motif has been modified as compared to a second αA motif of an unmodified plant LysM receptor polypeptide by insertion, deletion, or substitution of one or more amino acids, two or more amino acids, three or more amino acids, four or more amino acids, five or more amino acids, six or more amino acids, all seven amino acids, or wherein the first juxtamembrane domain lacks the first αA motif and the second αA motif is inserted into the corresponding position in the first juxtamembrane domain. Another aspect of the disclosure includes a modified plant non-NFR5 LysM receptor polypeptide engineered for NFR5 nodulation signaling including a first juxtamembrane domain (i) including a first αA motif, wherein the first αA motif was modified by substitution of one or more amino acids in the first αA motif with the corresponding amino acids from (1) a second αA motif from an NFR5 LysM receptor polypeptide, (2) found in an αA motif consensus sequence, or (ii) lacking the first αA motif, wherein the second αA motif from an NFR5 LysM receptor polypeptide is inserted into the first juxtamembrane domain. In an additional embodiment of this aspect, substitution includes deletion of an amino acid not present in the NFR5 αA motif, and insertion of an amino acid found in the NFR5 αA motif but not in the non-NFR5 αA motif. A further aspect of the disclosure includes a modified plant NFR5 LysM receptor polypeptide engineered for reduced NFR5 nodulation signaling including a first juxtamembrane domain including a first αA motif, wherein the first αA motif was modified by (i) substitution of one or more amino acids in the first αA motif with the corresponding amino acids from a second αA motif from a non-NFR5 LysM receptor polypeptide, or (ii) substitution of one or more amino acids in the first αA motif with an corresponding amino acid not found in an αA motif consensus sequence. Yet another embodiment of this aspect includes a modified plant LysM receptor polypeptide with altered signaling including a first juxtamembrane domain (i) including a first αA motif, wherein the first αA motif was modified by substitution of one or more amino acids in the first αA motif with the corresponding amino acids from a second αA motif from a LysM receptor polypeptide with different signaling, or (ii) lacking the first αA motif, wherein the second αA motif from a LysM receptor polypeptide with different signaling is inserted into the first juxtamembrane domain.

In a further embodiment of this aspect, the first αA motif, the second αA motif, or both are selected from a polypeptide with 70% identity, 75% identity, 80% identity, 85% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the first αA motif, the second αA motif, or both include or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or correspond to amino acids T286-S292 when aligned to SEQ ID NO: 8 (SEQ ID NO: 159). In a further embodiment of this aspect, which may be combined with any of the preceding embodiments, the first αA motif, the second αA motif, or both include the αA motif consensus sequence including X₁X₂X₃X₄LLX₅, where X₁ is T, I, G, or K; X₂ is A, Q, P, or G; X₃ is D or G; X₄ is K or E; X₅ is S, T, or P. In still another embodiment of this aspect, which may be combined with any of the preceding embodiments, the modification includes disruption of the αA motif, wherein disruption of the αA motif includes removing, replacing, or substituting one or more residues, two or more residues, three or more residues, four or more residues, five or more residues, six or more residues, or all seven residues within the αA motif. An additional embodiment of this aspect, which may be combined with any of the preceding embodiments, includes the modified plant LysM receptor polypeptide being a plant NFR5 LysM receptor polypeptide. In a further embodiment of this aspect, which may be combined with any of the preceding embodiments, the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In yet another embodiment of this aspect, which may be combined with any of the preceding embodiments that have a NFR5 LysM receptor polypeptide, the modified plant NFR5 LysM receptor polypeptide has reduced NFR5 nodulation signaling, as compared to the unmodified plant NFR5 LysM receptor polypeptide; the modified plant non-NFR5 LysM receptor polypeptide is able to initiate NFR5 nodulation signaling as compared to the unmodified plant non-NFR5 LysM receptor polypeptide; or the modified plant LysM receptor polypeptide is able to initiate different signaling as compared to the unmodified plant LysM receptor polypeptide. Altered nodulation signaling or different downstream symbiosis signaling readouts include nodule organogenesis, infection threads, root hair curling/swelling, calcium spiking, or transcriptional markers (e.g., NIN). Methods to assay these readouts are presented in the Examples. In still another embodiment of this aspect, which may be combined with any of the preceding embodiments that have a NFR5 LysM receptor polypeptide, the modified αA motif is selected from a polypeptide with 70% identity, 75% identity, 80% identity, 85% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the modified αA motif includes or corresponds to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or corresponds to amino acids T286-S292 when aligned to SEQ ID NO: 8 (SEQ ID NO: 159). In yet another embodiment of this aspect, which may be combined with any of the preceding embodiments, the plant LysM receptor polypeptide is an immunity pseudokinase-type receptor. In some embodiments, the immunity pseudokinase-type receptor is a LYS13/14-type receptor.

A further aspect of the disclosure includes a modified plant LysM receptor polypeptide including a first juxtamembrane domain including a first αA′ motif, wherein the first αA′ motif has been modified as compared to a second αA′ motif of an unmodified plant LysM receptor polypeptide by insertion, deletion, or substitution of one or more amino acids, two or more amino acids, three or more amino acids, four or more amino acids, five or more amino acids, six or more amino acids, all seven amino acids, or wherein the first juxtamembrane domain lacks the first αA′ motif and the second αA′ motif is inserted into the corresponding position in the first juxtamembrane domain. Another aspect of the disclosure includes a modified plant non-NFR5 LysM receptor polypeptide engineered for NFR5 nodulation signaling including a first juxtamembrane domain (i) including a first αA′ motif, wherein the first αA′ motif was modified by substitution of one or more amino acids in the first αA′ motif with the corresponding amino acids from (1) a second αA′ motif from an NFR5 LysM receptor polypeptide, (2) found in an αA′ motif consensus sequence, or (ii) lacking the first αA′ motif, wherein the second αA′ motif from an NFR5 LysM receptor polypeptide is inserted into the first juxtamembrane domain. In an additional embodiment of this aspect, substitution includes deletion of an amino acid not present in the NFR5 αA′ motif, and insertion of an amino acid found in the NFR5 αA′ motif but not in the non-NFR5 αA′ motif. A further aspect of the disclosure includes a modified plant NFR5 LysM receptor polypeptide engineered for reduced NFR5 nodulation signaling including a first juxtamembrane domain including a first αA′ motif, wherein the first αA′ motif was modified by (i) substitution of one or more amino acids in the first αA′ motif with the corresponding amino acids from a second αA′ motif from a non-NFR5 LysM receptor polypeptide, or (ii) substitution of one or more amino acids in the first αA′ motif with an corresponding amino acid not found in an αA′ motif consensus sequence. Yet another embodiment of this aspect includes a modified plant LysM receptor polypeptide with altered signaling including a first juxtamembrane domain (i) including a first αA′ motif, wherein the first αA′ motif was modified by substitution of one or more amino acids in the first αA′ motif with the corresponding amino acids from a second αA′ motif from a LysM receptor polypeptide with different signaling, or (ii) lacking the first αA′ motif, whercin the second αA′ motif from a LysM receptor polypeptide with different signaling is inserted into the first juxtamembrane domain.

In a further embodiment of this aspect, the first αA′ motif, the second αA′ motif, or both are selected from a polypeptide with 70% identity, 75% identity, 80% identity, 85% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In an additional embodiment of this aspect, the first αA′ motif, the second αA′ motif, or both are selected, include, or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In yet another embodiment of this aspect, which may be combined with any of the preceding embodiments, the first αA′ motif, the second αA′ motif, or both are selected, include, or correspond to X₆VSX₇X₈X₉X₁₀, where X₆ is G or S; X₇ is G, S, E, D, or Q; X₈ is Y or F; X₉ is V, L, or I; X₁₀ is S, G, or D. In still another embodiment of this aspect, which may be combined with any of the preceding embodiments, the modification includes disruption of the αA′ motif, wherein disruption of the αA′ motif includes removing, replacing, or substituting one or more residues, two or more residues, three or more residues, four or more residues, five or more residues, six or more residues, or all seven residues within the αA′ motif. An additional embodiment of this aspect, which may be combined with any of the preceding embodiments, includes the modified plant LysM receptor polypeptide being a plant NFR5 LysM receptor polypeptide. In a further embodiment of this aspect, which may be combined with any of the preceding embodiments, the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In yet another embodiment of this aspect, which may be combined with any of the preceding embodiments that have a NFR5 LysM receptor polypeptide, the modified plant NFR5 LysM receptor polypeptide has reduced NFR5 nodulation signaling, as compared to the unmodified plant NFR5 LysM receptor polypeptide; the modified plant non-NFR5 LysM receptor polypeptide is able to initiate NFR5 nodulation signaling as compared to the unmodified plant non-NFR5 LysM receptor polypeptide; or the modified plant LysM receptor polypeptide is able to initiate different signaling as compared to the unmodified plant LysM receptor polypeptide. Altered nodulation signaling or different downstream symbiosis signaling readouts include nodule organogenesis, infection threads, root hair curling/swelling, calcium spiking, or transcriptional markers (e.g., NIN). Methods to assay these readouts are presented in the Examples. In still another embodiment of this aspect, which may be combined with any of the preceding embodiments that have a NFR5 LysM receptor polypeptide, the modified αA′ motif is selected from a polypeptide with 70% identity, 75% identity, 80% identity, 85% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In a further embodiment of this aspect, the modified αA motif includes or corresponds to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In yet another embodiment of this aspect, which may be combined with any of the preceding embodiments, the plant LysM receptor polypeptide is an immunity pseudokinase-type receptor. In some embodiments, the immunity pseudokinase-type receptor is a LYS13/14-type receptor.

In a further embodiment of the disclosure, which may be combined with any of the preceding embodiments that have a modified plant LysM receptor polypeptide including a modified αA motif, the modified plant LysM receptor polypeptide further includes a modified αA′ motif. In a further embodiment of the disclosure, which may be combined with any of the preceding embodiments that have a modified plant LysM receptor polypeptide including a modified αA′ motif, the modified plant LysM receptor polypeptide further includes a modified αA motif.

A further aspect of the disclosure includes methods of identifying a plant LysM receptor polypeptide able to initiate a downstream symbiosis pathway, by: (a) providing a polypeptide sequence or a polypeptide model of a plant NFR5 receptor polypeptide juxtamembrane domain or an αA motif portion thereof and a candidate plant LysM receptor polypeptide; and (b) aligning the candidate plant LysM receptor polypeptide to the polypeptide sequence or the polypeptide model. In a further embodiment of this aspect, the plant LysM receptor polypeptide is able to initiate a downstream symbiosis pathway if it aligns with the αA motif of the NFR5 polypeptide juxtamembrane domain or is structurally similar to the αA motif of the NFR5 polypeptide juxtamembrane domain. In a further embodiment of this aspect, which may be combined with any of the preceding embodiments that has a NFR5 LysM receptor polypeptide, the plant NFR5 receptor αA motif includes SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 159.

A further aspect of the disclosure includes methods of identifying a plant LysM receptor polypeptide able to initiate a downstream symbiosis pathway, by: (a) providing a polypeptide sequence or a polypeptide model of a plant NFR5 receptor polypeptide juxtamembrane domain or an αA′ motif portion thereof and a candidate plant LysM receptor polypeptide; and (b) aligning the candidate plant LysM receptor polypeptide to the polypeptide sequence or the polypeptide model. In a further embodiment of this aspect, the plant LysM receptor polypeptide is able to initiate a downstream symbiosis pathway if it aligns with the αA′ motif of the NFR5 polypeptide juxtamembrane domain or is structurally similar to the αA′ motif of the NFR5 polypeptide juxtamembrane domain. In a further embodiment of this aspect, which may be combined with any of the preceding embodiments that has a NFR5 LysM receptor polypeptide, the plant NFR5 receptor αA′ motif includes SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160.

A further aspect of the disclosure includes a modified plant NFR5 LysM receptor polypeptide including a first intracellular domain including a first juxtamembrane domain and a first kinase domain, wherein the first intracellular domain was modified by substituting one or more amino acids in the first intracellular domain with the corresponding amino acids from a second intracellular domain including a second juxtamembrane domain and a second kinase domain. In an additional embodiment of this aspect, the first juxtamembrane domain includes a first αA motif, and wherein the second juxtamembrane domain includes a second αA motif. In a further embodiment of this aspect, the first αA motif, the second αA motif, or both are selected from a polypeptide with 70% identity, 75% identity, 80% identity, 85% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the first αA motif, the second αA motif, or both include or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or correspond to amino acids T286-S292 when aligned to SEQ ID NO: 8 (SEQ ID NO: 159). In still another embodiment of this aspect, which may be combined with any of the preceding embodiments, the first intracellular domain is from a first NFR5 LysM receptor polypeptide of a first plant species, and wherein the second intracellular domain is from a second NFR5 LysM receptor polypeptide of a second plant species.

Yet another aspect of the disclosure includes a modified plant NFR5 LysM receptor polypeptide including a first intracellular domain including a first juxtamembrane domain and a first kinase domain, wherein the first intracellular domain was modified by substituting one or more amino acids in the first intracellular domain with the corresponding amino acids from a second intracellular domain including a second juxtamembrane domain and a second kinase domain. In an additional embodiment of this aspect, the first juxtamembrane domain includes a first αA′ motif, and wherein the second juxtamembrane domain includes a second αA′ motif. In a further embodiment of this aspect, the first αA′ motif, the second αA′ motif, or both are selected from a polypeptide with 70% identity, 75% identity, 80% identity, 85% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In yet another embodiment of this aspect, the first αA motif, the second αA motif, or both include or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In still another embodiment of this aspect, which may be combined with any of the preceding embodiments, the first intracellular domain is from a first NFR5 LysM receptor polypeptide of a first plant species, and wherein the second intracellular domain is from a second NFR5 LysM receptor polypeptide of a second plant species.

Still another aspect of the disclosure includes a modified plant NFR5 LysM receptor polypeptide including a first intracellular domain including a first juxtamembrane domain and a first kinase domain, wherein the first intracellular domain was modified by substituting one or more amino acids in the first intracellular domain with the corresponding amino acids from a second intracellular domain including a second juxtamembrane domain and a second kinase domain. In an additional embodiment of this aspect, the first juxtamembrane domain includes a first αA and a first αA′ motif, and wherein the second juxtamembrane domain includes a second αA motif and a second αA′ motif. In a further embodiment of this aspect, the first αA motif, the second αA motif, or both are selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the first αA motif, the second αA motif, or both include or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or correspond to amino acids T286-S292 when aligned to SEQ ID NO: 8 (SEQ ID NO: 159). In a further embodiment of this aspect, the first αA′ motif, the second αA′ motif, or both are selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In yet another embodiment of this aspect, the first αA′ motif, the second αA′ motif, or both include or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In still another embodiment of this aspect, which may be combined with any of the preceding embodiments, the first intracellular domain is from a first NFR5 LysM receptor polypeptide of a first plant species, and wherein the second intracellular domain is from a second NFR5 LysM receptor polypeptide of a second plant species.

In yet another embodiment of any of the preceding aspects that include a first intracellular domain from a first NFR5 LysM receptor polypeptide of a first plant species and a second intracellular domain from a second NFR5 LysM receptor polypeptide of a second plant species, the first plant species is a legume plant species, and wherein the second plant species is a non-legume plant species; or wherein the first plant species is a non-legume plant species, and the second plant species is a legume plant species. In a further embodiment of this aspect, the legume plant species is selected from the group of bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), pea (e.g., Pisum spp., Pisum sativum var. sativum, Pisum sativum var. arvense), chickpea (e.g., garbanzo, Bengal gram, Cicer arietinum), cowpea (e.g., Vigna unguiculata), pigeon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), or legume trees (e.g., locust trees, Gleditsia spp., Robinia spp., Kentucky coffeetree, Gymnocladus dioicus, Acacia spp., Laburnum spp., Wisteria spp.). In another embodiment of this aspect, the non-legume plant species is selected from the group of cassava (e.g., manioc, yucca, Manihot esculenta), yam (e.g., Dioscorea rotundata, Dioscorea alata, Dioscorea trifida, Dioscorea sp.), sweet potato (e.g., Ipomoea batatas), taro (e.g., Colocasia esculenta), oca (e.g., Oxalis tuberosa), corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tef), oat (e.g., Avena sativa), triticale (e.g., Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), wheat (e.g., common wheat, spelt, durum, einkorn, emmer, kamut, Triticum aestivum, Triticum spelta, Triticum durum, Triticum urrartu, Triticum monococcum, Triticum turanicum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), apple (e.g., Malus domestica, Malus pumila, Pyrus malus), pear (e.g., Pyrus communis, Pyrus×bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria×ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), or hemp (e.g., cannabis, Cannabis sativa).

### Genetically Modified Plants and Related Methods

Some aspects of the disclosure include a modified plant or part thereof including the modified plant LysM receptor polypeptide of any one of the preceding embodiments. In a further embodiment of this aspect, the modified plant LysM receptor polypeptide includes a modified αA motif selected from a polypeptide with 70% identity, 75% identity, 80% identity, 85% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the modified αA motif includes or corresponds to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or corresponds to amino acids T286-S292 when aligned to SEQ ID NO: 8 (SEQ ID NO: 159). In a further embodiment of this aspect, which may be combined with any of the preceding embodiments, the modified αA motif includes the αA motif consensus sequence including X₁X₂X₃X₄LLX₅, where X₁ is T, I, G, or K; X₂ is A, Q, P, or G; X₃ is D or G; X₄ is K or E; X₅ is S, T, or P. In an additional embodiment of this aspect, which may be combined with any of the preceding embodiments, the modified plant LysM receptor polypeptide includes a modified αA′ motif selected from a polypeptide with 70% identity, 75% identity, 80% identity, 85% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In an additional embodiment of this aspect, the modified αA′ motif includes or corresponds to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In yet another embodiment of this aspect, which may be combined with any of the preceding embodiments, the modified αA′ motif includes the αA′ motif consensus sequence including X₆ VSX₇X₈X₉X₁₀, where X₆ is G or S; X₇ is G, S, E, D, or Q; X₈ is Y or F; X₉ is V, L, or I; X₁₀ is S, G, or D.

In still another embodiment of this aspect, which may be combined with any of the preceding embodiments, the plant part is a leaf, a stem, a root, a root primordia, a flower, a seed, a fruit, a kernel, a grain, a cell, or a portion thereof. In yet a further embodiment of this aspect, which may be combined with any of the preceding embodiments, the plant is selected from the group of cassava (e.g., manioc, yucca, Manihot esculenta), yam (e.g., Dioscorea rotundata, Dioscorea alata, Dioscorea trifida, Dioscorea sp.), sweet potato (e.g., Ipomoea batatas), taro (e.g., Colocasia esculenta), oca (e.g., Oxalis tuberosa), corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tef), oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), apple (e.g., Malus domestica, Malus pumila, Pyrus malus), pear (e.g., Pyrus communis, Pyrus×bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria×ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), pea (e.g., Pisum spp., Pisum sativum var. sativum, Pisum sativum var. arvense), chickpea (e.g., garbanzo, Bengal gram, Cicer arietinum), cowpea (e.g., Vigna unguiculata), pigeon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

Further aspects of the disclosure include methods of making the genetically modified plant or part thereof of any of the preceding embodiments, including introducing a genetic alteration to the plant or part thereof including a nucleic acid sequence encoding a heterologous plant LysM receptor polypeptide including a juxtamembrane domain including a modified αA motif. In a further embodiment of this aspect, the nucleic acid sequence is operably linked to a promoter. In an additional embodiment of this aspect, the promoter is a root specific promoter, an inducible promoter, a constitutive promoter, or a combination thereof. In still another embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR 1 promoter (SEQ ID NO:124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In a further embodiment of this aspect, the nucleic acid sequence is inserted into the genome of the plant so that the nucleic acid sequence is operably linked to an endogenous promoter. In another embodiment of this aspect, the endogenous promoter is a root specific promoter.

Additional aspects of the disclosure include methods of making the genetically modified plant or part thereof of any of the preceding embodiments, including genetically modifying the plant or part thereof by transforming the plant or part thereof with one or more gene editing components that target an endogenous nuclear genome sequence encoding an endogenous plant LysM receptor polypeptide including a juxtamembrane domain including an αA motif, wherein the αA motif is genetically modified by removing, replacing, or substituting one or more residues, two or more residues, three or more residues, four or more residues, five or more residues, six or more residues, or all seven residues within the αA motif. In some embodiments of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

Further aspects of the disclosure include methods of making the genetically modified plant or part thereof of any of the preceding embodiments, including introducing a genetic alteration to the plant or part thereof including a nucleic acid sequence encoding a heterologous plant LysM receptor polypeptide including a juxtamembrane domain including a modified αA′ motif. In a further embodiment of this aspect, the nucleic acid sequence is operably linked to a promoter. In an additional embodiment of this aspect, the promoter is a root specific promoter, an inducible promoter, a constitutive promoter, or a combination thereof. In still another embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR 1 promoter (SEQ ID NO:124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In a further embodiment of this aspect, the nucleic acid sequence is inserted into the genome of the plant so that the nucleic acid sequence is operably linked to an endogenous promoter. In another embodiment of this aspect, the endogenous promoter is a root specific promoter.

Additional aspects of the disclosure include methods of making the genetically modified plant or part thereof of any of the preceding embodiments, including genetically modifying the plant or part thereof by transforming the plant or part thereof with one or more gene editing components that target an endogenous nuclear genome sequence encoding an endogenous plant LysM receptor polypeptide including a juxtamembrane domain including an αA′ motif, wherein the αA′ motif is genetically modified by removing, replacing, or substituting one or more residues, two or more residues, three or more residues, four or more residues, five or more residues, six or more residues, or all seven residues within the αA′ motif. In some embodiments of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZEN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

In some embodiments, which may be combined with any of the preceding method embodiments, the modified plant LysM receptor polypeptide is a plant NFR5 LysM receptor polypeptide. An additional embodiment of this aspect includes the NFR5 receptor polypeptide being selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In a further embodiment of this aspect, which may be combined with any of the preceding embodiments that has a NFR5 LysM receptor polypeptide, the modified plant NFR5 LysM receptor polypeptide has reduced NFR5 nodulation signaling, as compared to the unmodified plant NFR5 LysM receptor polypeptide.

In some embodiments, which may be combined with any of the preceding method embodiments, the modified plant LysM receptor polypeptide is an immunity pseudokinase-type receptor. In some embodiments, the immunity pseudokinase-type receptor is a LYS13/14-type receptor.

In some embodiments, which may be combined with any of the preceding method embodiments, the αA motif and/or the modified αA motif is selected from a polypeptide with 70% identity, 75% identity, 80% identity, 85% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. In an additional embodiment of this aspect, the αA motif and/or the modified αA motif includes or corresponds to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or corresponds to amino acids T286-S292 when aligned to SEQ ID NO: 8 (SEQ ID NO: 159). In a further embodiment of this aspect, which may be combined with any of the preceding embodiments, the αA motif and/or the modified αA motif includes the αA motif consensus sequence including X₁X₂X₃X₄LLX₅, where X₁ is T, I, G, or K; X₂ is A, Q, P, or G; X₃ is D or G; X₄ is K or E; X₅ is S, T, or P.

In some embodiments, which may be combined with any of the preceding method embodiments, the αA′ motif and/or the modified αA′ motif is selected from a polypeptide with 70% identity, 75% identity, 80% identity, 85% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. In an additional embodiment of this aspect, the αA motif and/or the modified αA motif includes or corresponds to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160). In a further embodiment of this aspect, which may be combined with any of the preceding method embodiments, the αA′ motif and/or the modified αA′ motif includes the αA′ motif consensus sequence including X₆VSX₇X₈X₉X₁₀, where X₆ is G or S; X₇ is G, S, E, D, or Q; X₈ is Y or F; X₉ is V, L, or I; X₁₀ is S, G, or D.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

### Expression Vectors, Isolated DNA Molecules, or Recombinant Nucleic Acids; Cells, Compositions, or Kits Including the Same; and Related Methods

An additional aspect of the disclosure includes an expression vector, isolated DNA molecule, or recombinant nucleic acid including one or more nucleic acid sequences encoding a heterologous plant LysM receptor polypeptide including a juxtamembrane domain including a modified αA motif and/or modified αA′ motif, wherein the one or more nucleic acid sequences are operably linked to at least one expression control sequence. In a further embodiment of this aspect, the plant LysM receptor polypeptide is a NFR5 receptor polypeptide. In another embodiment of this aspect, the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In yet another embodiment of this aspect, the plant LysM receptor polypeptide is an immunity pseudokinase-type receptor. In some embodiments, the immunity pseudokinase-type receptor is a LYS13/14-type receptor. In still another embodiment of this aspect, which may be combined with any of the preceding embodiments, the at least one expression control sequence includes a promoter selected from the group of a root specific promoter, a constitutive promoter, or a combination thereof. In a further embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR1 promoter (SEQ ID NO: 124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In an additional embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

Further aspects of the disclosure include a bacterial cell or an Agrobacterium cell including the expression vector, isolated DNA molecule, or recombinant nucleic acid of any one of the preceding embodiments.

Other aspects of the disclosure include a genetically modified plant, plant part, plant cell, or seed including the expression vector, isolated DNA molecule, or recombinant nucleic acid of any one of the preceding embodiments.

Additional aspect of the disclosure include a composition or kit including the expression vector, isolated DNA molecule, or recombinant nucleic acid of any one of the preceding embodiments, the bacterial cell or the Agrobacterium cell of any one of the preceding embodiments, or the genetically modified plant, plant part, plant cell, or seed of any one of the preceding embodiments.

Some aspects of the disclosure include methods of reducing NFR5 nodulation signaling including: introducing a genetic alteration via an expression vector, isolated DNA molecule, or recombinant nucleic acid of any one of the preceding embodiments to a cell. A further embodiment of this aspect includes the plant being a plant cell.

### Heavy-Chain Variable Domain (VHH)

A camelid heavy-chain variable domain (VHH) (also known as Nanobody®, Ablynx), also known as a single domain antibody (sdAb), single variable domain antibody or a single monomeric variable antibody domain, recombinant variable domain of a heavy-chain-only antibody, target-binding fragment of a monoclonal antibody, a polyclonal VHH fragment antibody, a miniature engineered antibody, or a multiple antigen-binding fragment, is an antibody fragment consisting of a single monomeric variable antibody domain. Like a whole antibody, the VHH is able to bind selectively to a specific antigen. With a molecular weight of only 12-15 kDa, VHHs are much smaller than common whole antibodies (150-160 kDa). A VHH is a peptide chain of about 110 amino acids in length, comprising one variable domain (VH) of a heavy chain antibody. Unlike whole antibodies, VHHs do not show complement system triggered cytotoxicity because they lack an Fc region. Camelid- and fish-derived VHHs are able to bind to hidden antigens that are not accessible to whole antibodies, for example to the active sites of enzymes. A VHH can be obtained by immunization of sharks or camelids with the desired antigen and subsequent isolation of the mRNA coding for heavy chain antibodies. Camelids are members of the biological family Camelidae, the only living family in the suborder Tylopoda. Camels, dromedarics, Bactrian Camels, llamas, alpacas, vicunas, and guanacos are in this group. Alternatively, VHHs can be made by screening synthetic libraries.

“VHH domains”, also known as VHHs, VHH domains, VHH antibody fragments, and VHH antibodies, were originally described as the antigen binding immunoglobulin (variable) domain of “heavy chain antibodies” (i.e., of “antibodies devoid of light chains”; Hamers-Casterman et al. (1993) Nature 363:446-448). The term “VHH domain” was chosen to distinguish these variable domains from the heavy chain variable domains that are present in conventional 4-chain antibodies (which are referred to hercin as “VH domains”) and from the light chain variable domains that are present in conventional 4-chain antibodies (which are referred to herein as “VL domains”). For a further description of VHHs, reference is made to the review article by Muyldermans (Reviews in J. Biotechnol. 74:277-302, 2001), as well as to the following patent applications, which are mentioned as general background art: WO 94/04678, WO 95/04079, WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, VHHs (in particular VHH sequences and partially humanized VHHs) can in particular be characterized by the presence of one or more “Hallmark residues” in one or more of the framework sequences. A further description of the VHHs, including humanization and/or camelization of VHHs, as well as other modifications, parts or fragments, derivatives or “VHH fusions”, multivalent constructs (including some non-limiting examples of linker sequences) and different modifications to increase the half-life of the VHHs and their preparations can be found, e.g., in WO 08/101985 and WO 08/142164

### LysM Receptors and NFR5

LysM receptors may be defined as proteins that contain three tandem LysM domains in their extracellular region, namely LysM1, LysM2, and LysM3, which are present in this order on the protein sequence and separated by CxC motifs. The LysMI domain is located toward the N-terminal end of the protein sequence, and is preceded by an N-terminal signal peptide. Moving into the cell, the LysM domains are followed by a single-pass transmembrane domain, a juxtamembrane domain, and an intracellular kinase or pseudokinase domain. Most plant LysM receptors contain an intracellular kinase domain, while some, including NFR5, contain an intracellular pseudokinase domain.

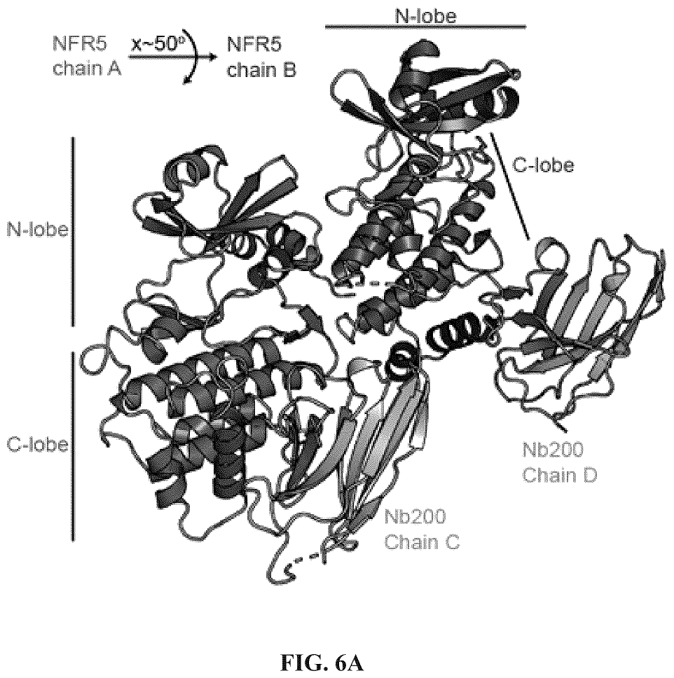

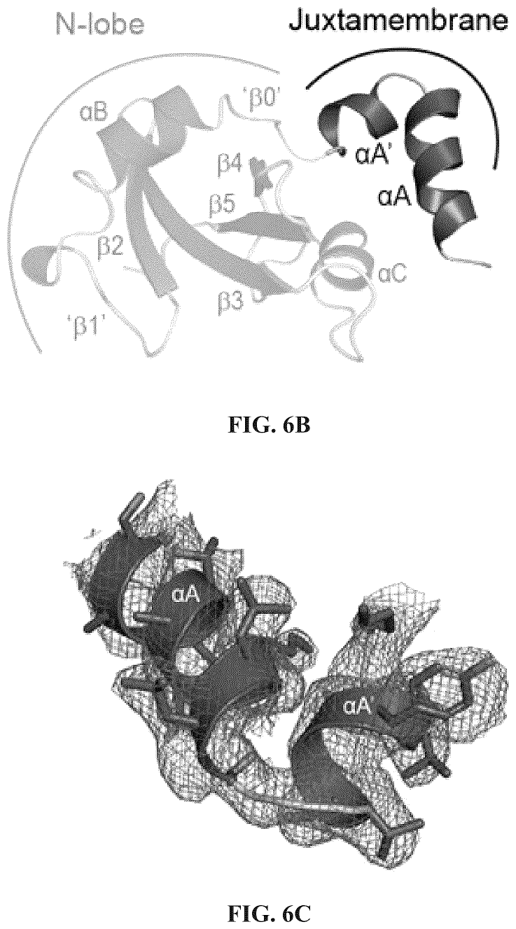

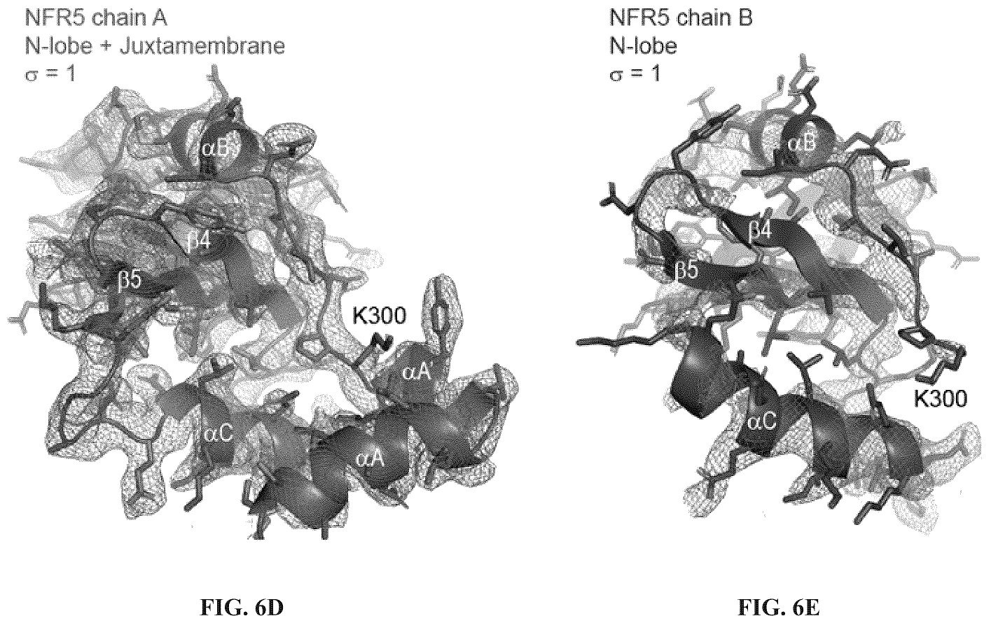

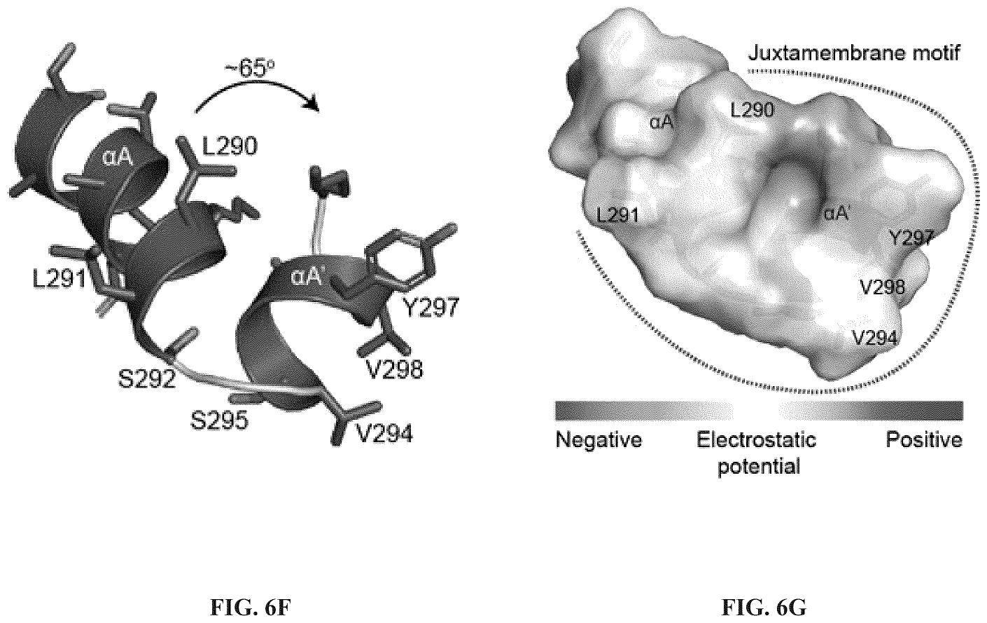

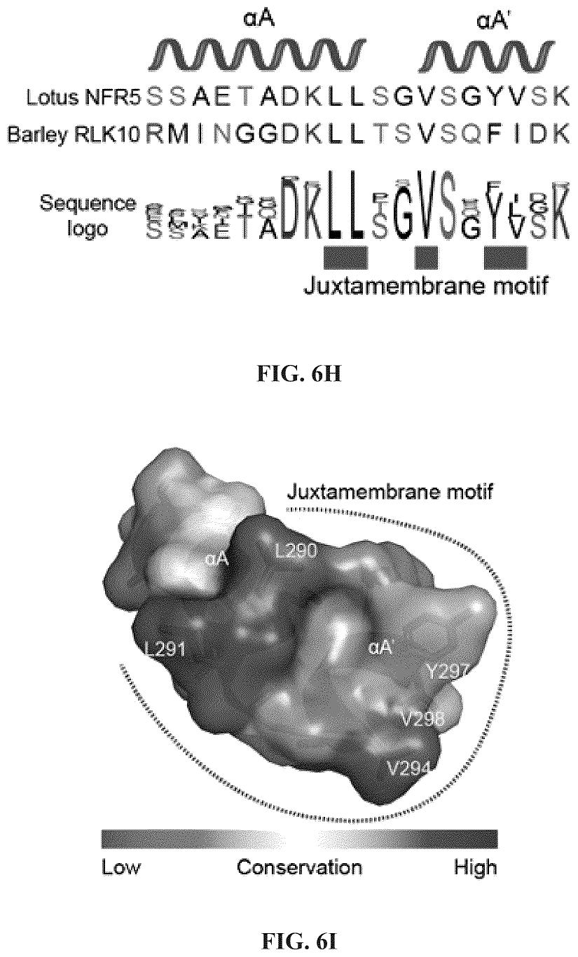

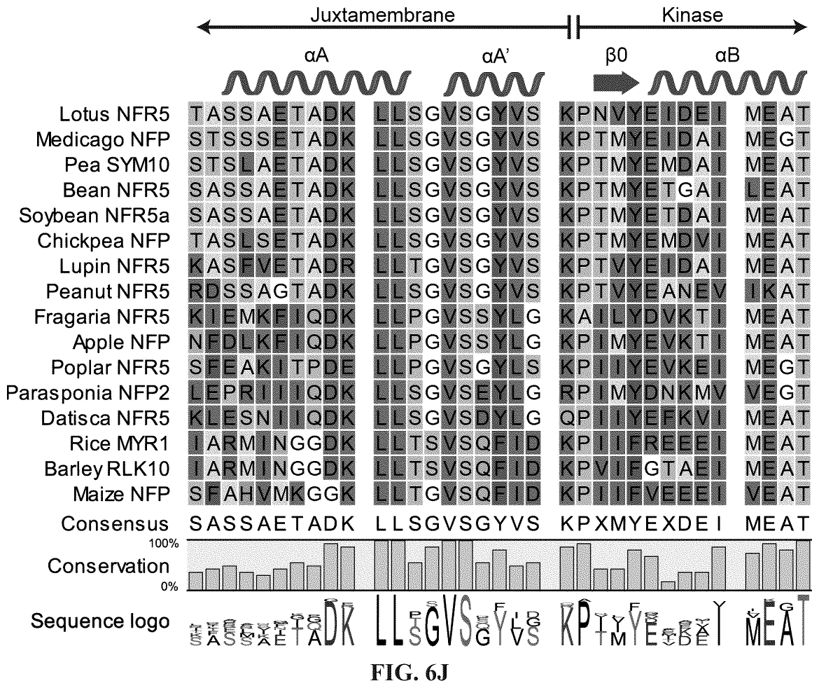

The present disclosure provides the crystal structure of the intracellular domain of the NFR5 receptor, including the juxtamembrane domain and the protein kinase domain. The structure revealed that the juxtamembrane of NFR5 contained two alphα-helices, αA and αA′, with αA containing a solvent exposed hydrophobic motif. The hydrophobic motif resembled a known EGFR juxtamembrane dimerization motif, and minimal substitution mutations in this motif were found to abolish NFR5 functionality in planta. Further, this motif was found to be conserved across NFR5-type receptors in different plant species (FIG. 6J).

Lohmann et al., 2010 presents additional characteristics of NFR5-type receptors (Lohmann G V, Shimoda Y, Nielsen M W, Jørgensen F G, Grossmann C, Sandal N, Sørensen K, Thirup S, Madsen L H, Tabata S, Sato S, Stougaard J, Radutoiu S. Evolution and regulation of the Lotus japonicus LysM receptor gene family. Mol Plant Microbe Interact. (2010) 23 (4), 510-21). In short, NFR5-type receptors are translated from single-exon genes. Further, NFR5-type receptors contain intracellular pseudokinases where subdomain I and VII/VIII are missing, or the conserved residues are substituted. NFR5-type receptors may also be referred to as NFP-type receptors or Lys11-type receptors, as each of Lotus japonicus NFR5, Medicago truncatula NFP, and Lotus japonicus Lys11 belong to this receptor type.

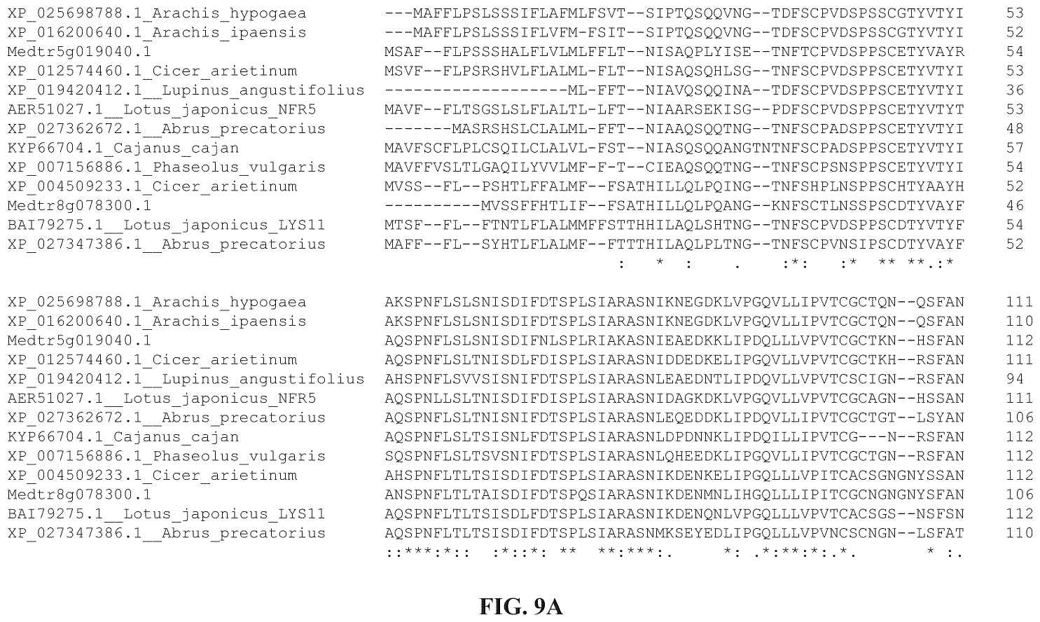

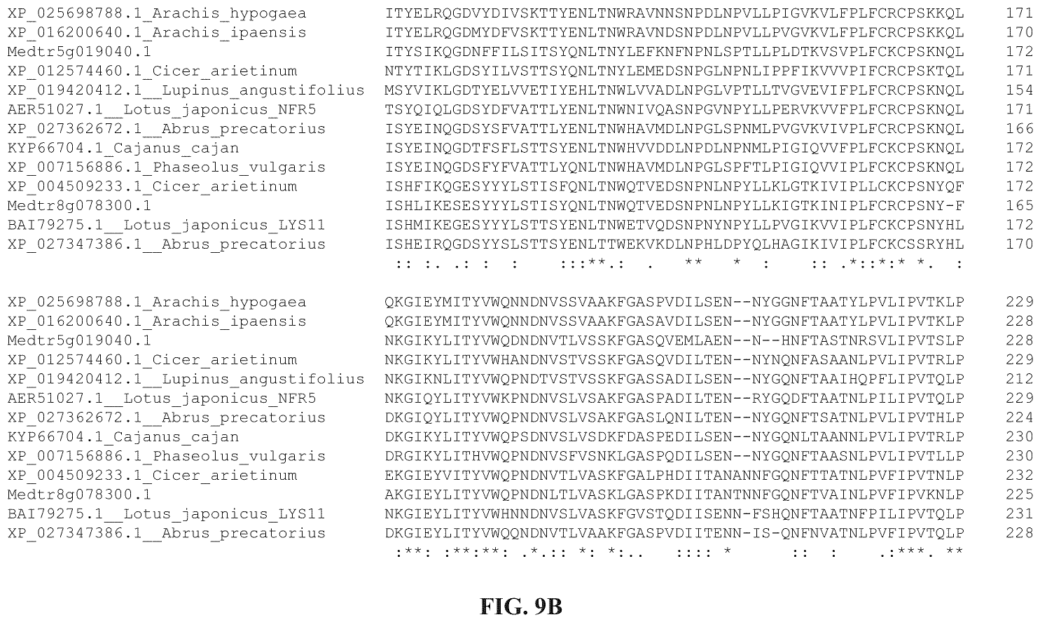

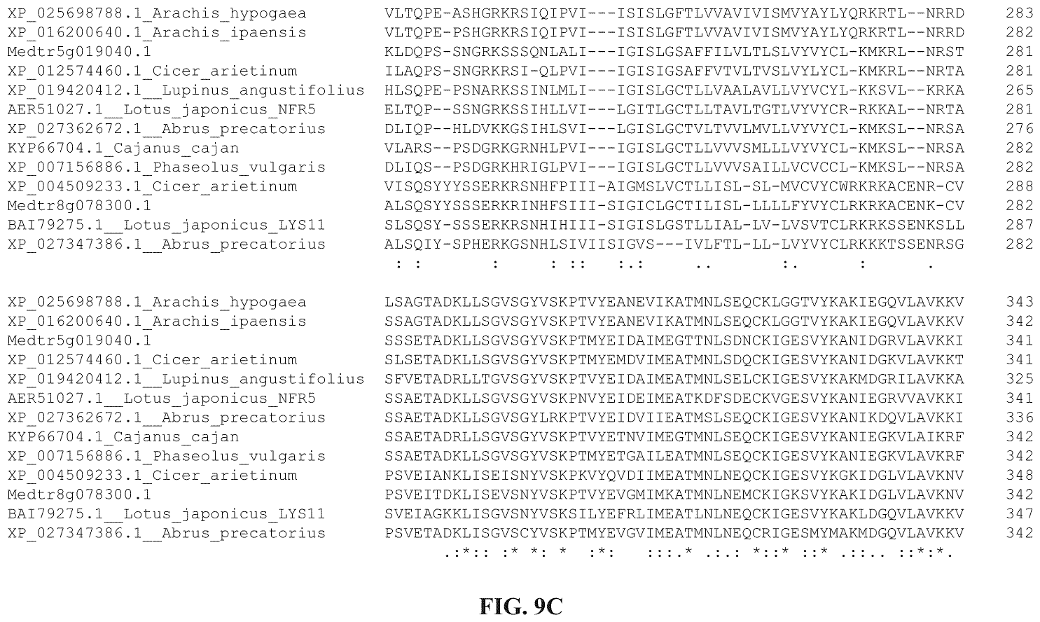

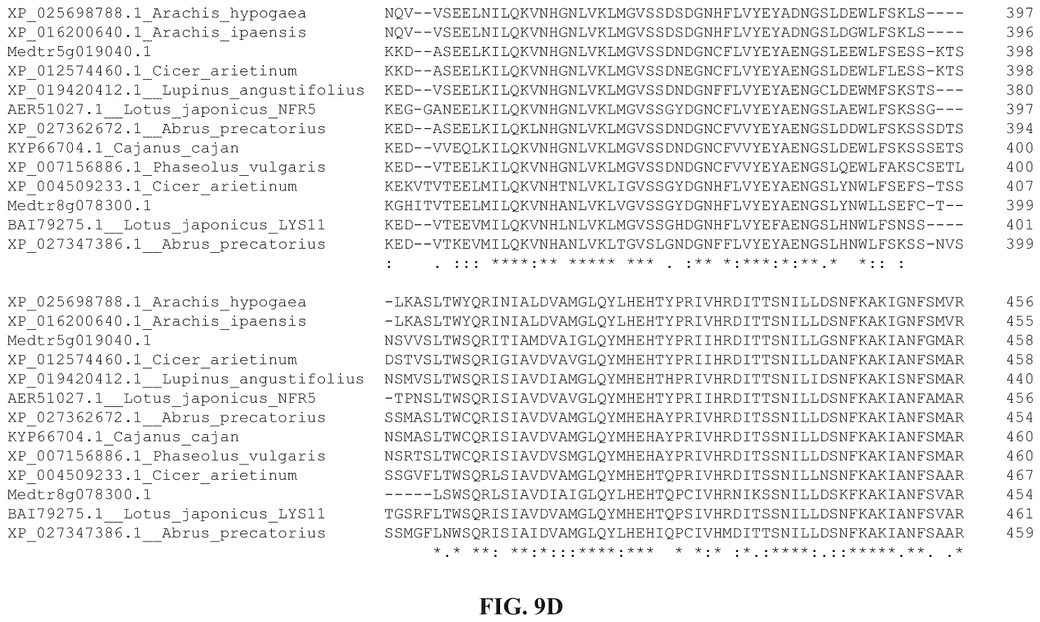

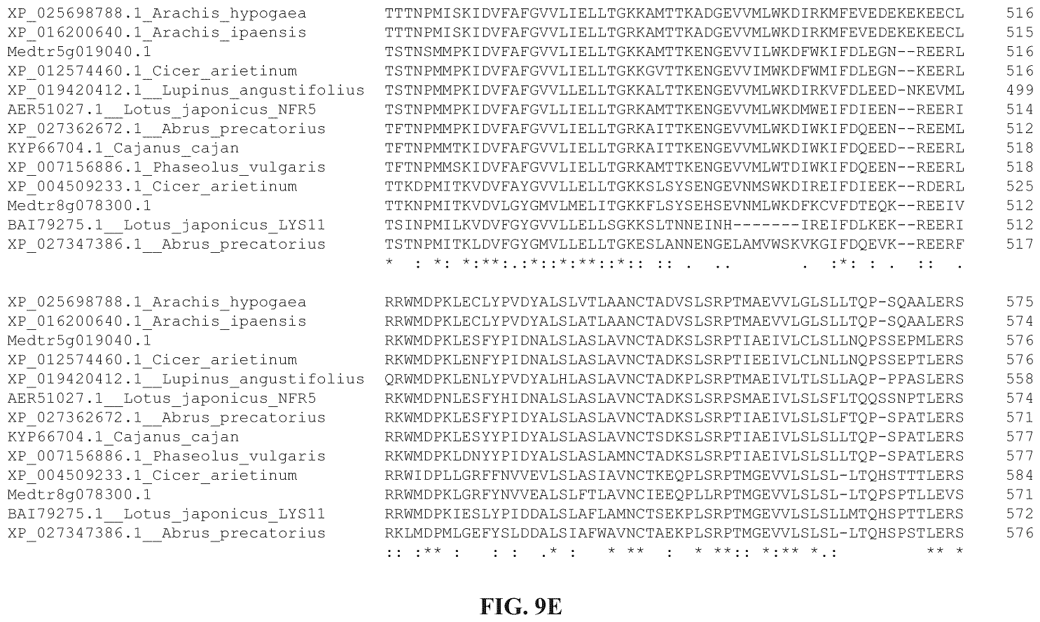

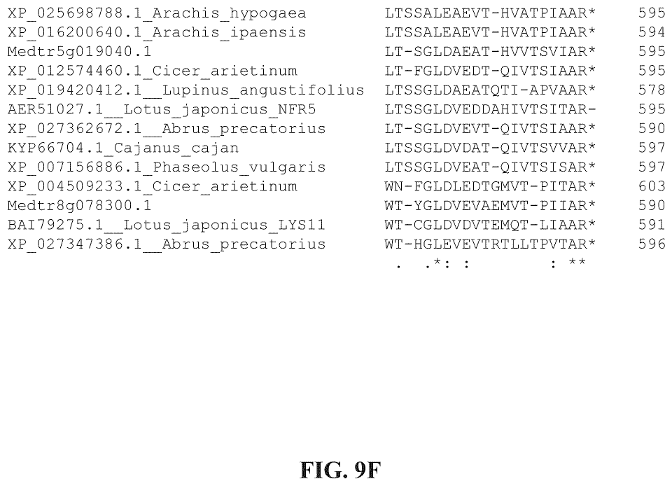

An alignment of NFR5-type LysM receptors is shown in FIGS. 9A-9F. Exemplary NFR5-type LysM Nod factor receptors include SEQ ID NO: 9, SEQ ID NO: 10, SEQ ID NO: 5, SEQ ID NO: 3, SEQ ID NO: 8, SEQ ID NO: 4, SEQ ID NO: 12, SEQ ID NO: 13, SEQ ID NO: 11, SEQ ID NO: 6, SEQ ID NO: 7, SEQ ID NO: 1, SEQ ID NO: 2, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. NFR5 receptors are also described in Gough et al., (2018) Evolutionary History of Plant LysM Receptor Proteins Related to Root Endosymbiosis, Front. Plant Sci. 9:923.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self-or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), Genotyping by Sequencing (GbS), and Next-generation Sequencing (NGS).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (lib[dot]dr[dot]iastate[dot]edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib.dr.iastate.edu/agron_books/1), which are herewith incorporated by reference.

### Molecular Biological Methods to Produce Transgenic Plant Cells, Plant Parts, and Plants

One aspect of the present disclosure provides transgenic plant cells, plant parts, or plants including a modified plant LysM receptor polypeptide including a juxtamembrane domain including an αA motif, and optionally further including an αA′ motif. In a further embodiment of this aspect, the plant LysM receptor polypeptide is a NFR5 receptor. In addition, the present disclosure provides isolated DNA molecules of vectors and gene editing components used to produce transgenic plants of the present disclosure.

Exemplary αA motif sequences include or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159. The αA motif consensus sequence is X₁X₂X₃X₄LLX₅, where X₁ is T, I, G, or K; X₂ is A, Q, P, or G; X₃ is D or G; X₄ is K or E; X₅ is S, T, or P.

Exemplary αA′ motif sequences include or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160. The αA′ motif consensus sequence is X₆ VSX₇X₈X₉X₁₀, where X₆ is G or S; X₇ is G, S, E, D, or Q; X₈ is Y or F; X₉ is V, L, or I; X₁₀ is S, G, or D.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application, and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors cach contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (Kay et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466) the maize ubiquitin promoter (Christensen & Quail, Transgenic Res, 5, 213-8, 1996), the polyubiquitin promoter (Ljubql, Mackawa et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in root epidermal cells or root cortex cells. In preferred embodiments, LysM receptor promoters will be used. Non-limiting examples include NFR1 promoters, NFR5 promoters, LYK3 promoters, NFP promoters, the Lotus japonicus NFR5 promoter (SEQ ID NO: 24), the Lotus japonicus NFR 1 promoter (SEQ ID NO: 124), the Medicago truncatula NFP promoter (SEQ ID NO: 25), the Lotus japonicus CERK6 promoter (SEQ ID NO: 26), and the Medicago truncatula LYK3 promoter (SEQ ID NO: 27). In additional preferred embodiments, root specific promoters will be used. Non-limiting examples include the promoter of the maize metallothionein (De Framond et al, FEBS 290, 103.-106, 1991 Application EP 452269), the chitinase promoter (Samac et al. Plant Physiol 93, 907-914, 1990), the glutamine synthetase soybean root promoter (Hirel et al. Plant Mol. Biol. 20, 207-218, 1992), the RCC3 promoter (PCT Application WO 2009/016104), the rice antiquitin promoter (PCT Application WO 2007/076115), the LRR receptor kinase promoter (PCT application WO 02/46439), the maize ZRP2 promoter (U.S. Pat. No. 5,633,363), the tomato LeExtl promoter (Bucher et al. Plant Physiol. 128, 911-923, 2002), and the Arabidopsis pCO2 promoter (Heidstra et al, Genes Dev. 18, 1964-1969, 2004). These plant promoters can be combined with enhancer elements, they can be combined with minimal promoter elements, or can comprise repeated elements to ensure the expression profile desired.

Examples of constitutive promoters that are often used in plant cells are the cauliflower mosaic (CaMV) 35S promoter (Kay et al. Science, 236, 4805, 1987), and various derivatives of the promoter, virus promoter vein mosaic cassava (International Application WO 97/48819), the maize ubiquitin promoter (Christensen & Quail, Transgenic Res, 5, 213-8, 1996), polyubiquitin (Ljubql, Mackawa et al. Mol Plant Microbe Interact. 21, 375-82, 2008) and Arabidopsis UBQ10 (Norris et al. Plant Mol. Biol. 21, 895-906, 1993).

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty: 2, Nucleic match: 1, Nucleic mismatch-3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit, the claimed disclosure.

### Example 1: A Heavy Chain Variable Domain (VHH) Facilitates the Structural Determination of the Signaling Competent NFR5 Intracellular Domain

The following example describes the isolation and characterization of the NFR5-intracellular domain and its role in signaling.

### Introduction

The LysM receptor kinase NFR5 is required for the perception of rhizobial nod factors and the subsequent initiation of root nodule symbiosis between legumes and nitrogen-fixing rhizobia bacteria (Radutoiu et al. (2003), Science 425, 585-592; Madsen et al. (2003), Science 425, 637-640; Radutoiu et al. (2007), EMBO J 26, 3923-3935; Rübsam et al. (2023), Science 379, 272-277). Lotus japonicus (hereafter Lotus) NFR5 is composed of an extracellular domain of three lysin-motifs (LysM), which selectively binds the nod factor signal produced by the symbiont of Lotus, Mesorhizobium loti (Radutoiu et al. (2007), EMBO J 26, 3923-3935; Broghammer et al. (2012), Proc Natl Acad Sci USA 109, 13859-13864; Bozsoki et al. (2020), Science 369, 663-670; Gysel et al. (2021), Proc Natl Acad Sci USA 118). NFR5 is embedded in the plant plasma membrane via a single transmembrane helix and on the intracellular side contains an N-terminal juxtamembrane part followed by a protein kinase domain and a C-terminal tail region (Madsen et al. (2003), Science 425, 637-640). The NFR5 kinase is degenerated in several conserved motifs and is consequently a catalytically inactive pseudokinase. In contrast, the receptor partner NFR1, contains a conventional protein kinase and NFR1 phosphorylation activity is required for symbiosis signaling (Radutoiu et al. (2003), Science 425, 585-592; Madsen et al. (2011), Plant J 65, 404-417). Despite the lack of catalytic activity, the intracellular domain of NFR5 is required for establishing root nodule symbiosis (Miyata et al. (2016), Plant Cell Physiol 57, 2283-2290). While protein kinase phosphorylation signaling is well described in biology, the signaling mechanisms for catalytically inactive pseudokinases are less understood. However, NFR5-type receptors are prevalent throughout plants and several studies have demonstrated the functional importance of this class of receptors in establishing symbioses between plants and microbes (Madsen et al. (2003), Science 425, 637-640; Amor et al. (2003), The Plant Journal 34, 495-506; Op Den Camp et al. (2011), Science 331, 909-912; Buendia et al. (2016), New Phytol 210, 184-195; He et al. (2019), Mol Plant 12, 1561-1576). This example and the following examples describe insights into the signaling function of NFR5-type receptors. A crystal structure of the Lotus NFR5 intracellular domain is provided that contains the core kinase and two juxtamembrane α-helices. NFR5 was found to readily self-interact and an exposed hydrophobic motif in the juxtamembrane α-helices was found to be important for this self-interaction. Analyses revealed that the juxtamembrane motif is conserved in NFR5-type receptors and is essential for symbiosis signaling in both Lotus NFR5 and barley RLK10.

### Materials and Methods

### Protein Expression and Purification

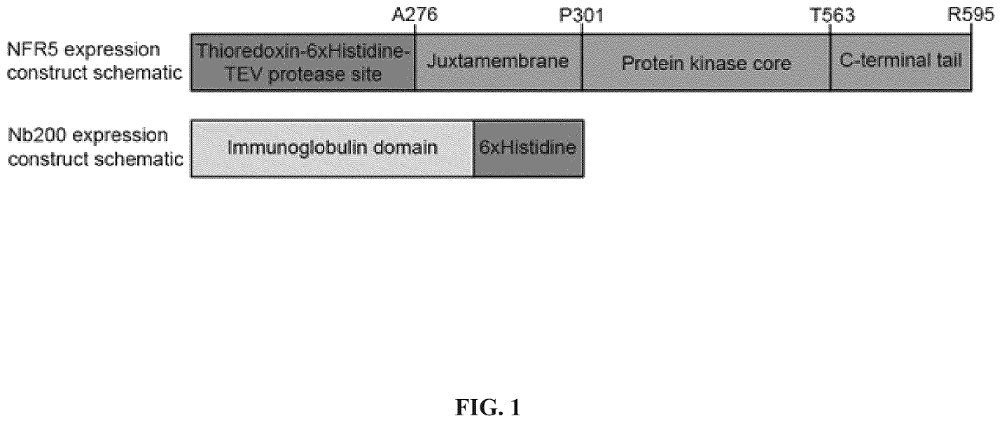

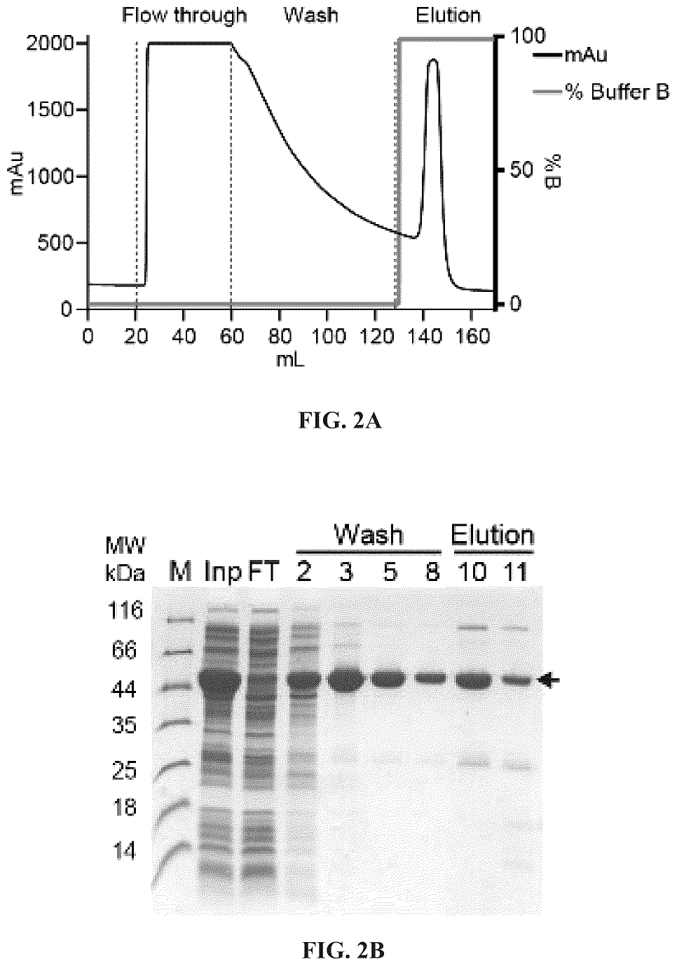

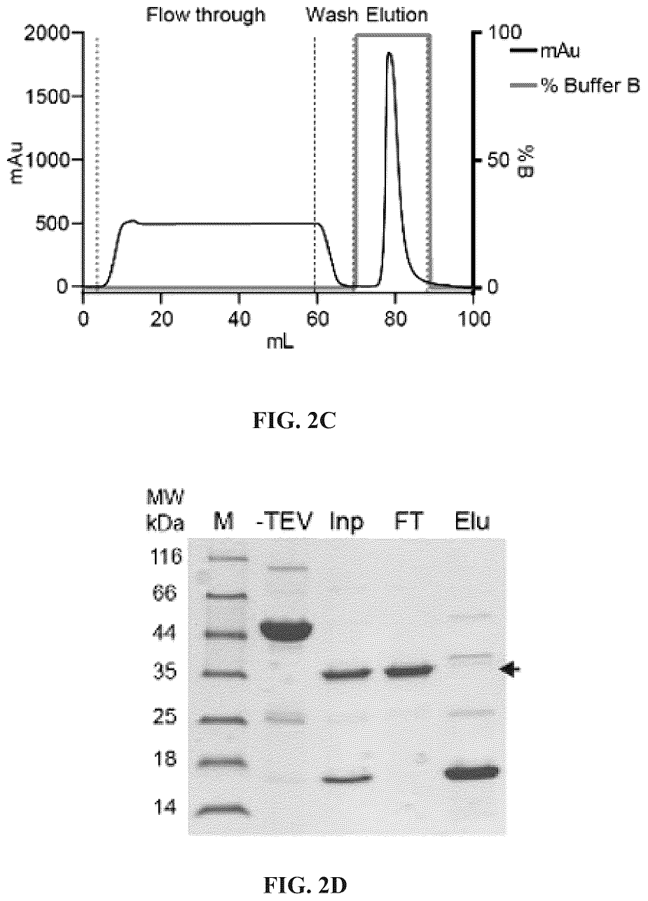

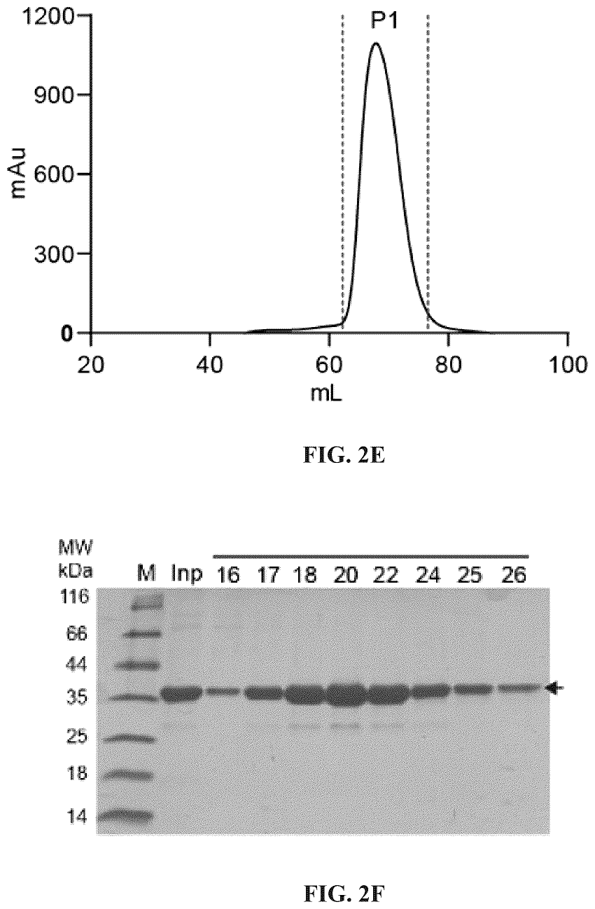

NFR5 (residues 276-595; SEQ ID NO: 146) and NFR 1 (residues 263-599; SEQ ID NO: 152) expression constructs were cloned in a pET32 Ek/LIC vector backbone containing an N-terminal thioredoxin and 6×Histidine tag. A tobacco etch virus (TEV) protease site was further introduced to allow fusion-tag removal (SEQ ID NO: 123). Heavy chain variable domains (VHHs) were cloned into the pET-22b(+) vector containing a C-terminal 6×Histidine tag (FIG. 1). Rosetta II and LOBSTR (Andersen et al. 2013, Proteins 81 (11): 1857-1861) E. coli strains were used for NFR5 and VHH expression, respectively. E. coli were heat-shock transformed and plated on lysogeny broth (LB) agar (LA) supplemented with 100 μg/ml ampicillin (AMP) and 50 μg/ml chloramphenicol (CAM) and incubated overnight at 37° C. 200 mL LB supplemented with 100 μg/ml ampicillin (AMP) and 50 μg/ml chloramphenicol (CAM) was inoculated with a single colony and cultured overnight in a shaking incubator at 37° C., 180 RPM. 20 mL overnight culture was used to seed 2 L LB supplemented with 100 μg/ml ampicillin (AMP) and 50 μg/ml chloramphenicol (CAM), and E. coli were cultured at 37° C., 180 RPM until reaching OD₆₀₀=0.5-0.8. Protein expression was induced by addition of isopropylthio-β-galactoside (IPTG) to a final concentration of 0.4 mM and cultures were incubated overnight with shaking at 18° C., 180 RPM. Cells were pelleted by centrifugation at 5000 g, 4° C. for 15 minutes. Cell pellets were suspended in lysis buffer (50 mM Tris-HCl pH 8, 500 mM NαCl, 20 mM imidazole, 5 mM β-mercaptocthanol, 10% v/v glycerol, 1 mM benzamidine). E. coli suspensions were sonicated on ice three times for 5 minutes each time using a LABSONIC® M ultrasonic homogenizer (Sartorious). Lysate was cleared by centrifugation at 16000 g, 4° C. for 30 minutes. Supernatant was collected and applied to a lysis buffer-equilibrated Protino® Ni-NTA column (Machercy-Nagel) using an ÄKTA™ start system (Cytiva). After sample application the column was extensively washed in lysis buffer until UV₂₈₀ baseline and protein was eluted in 5 column volumes of Buffer B (50 mM Tris-HCl pH 8, 500 mM NαCl, 250 mM imidazole, 5 mM β-mercaptocthanol, 5% glycerol) (FIGS. 2A-2B). Protein was efficiently captured by the Ni-NTA column as was evident from the large peak produced during the clution step. A dragging shoulder was observed during the wash and indicated over-saturation of the column. Few contaminants were observed. Wash fractions 3-9 were sufficiently pure and were pooled with clution fractions 10-11. Protein was dialyzed overnight at 4° C. against dialysis buffer (50 mM Tris-HCl pH 8, 500 mM NαCl, 5 mM β-mercaptoethanol) using a Spectra/Por® 14 kDa MWCO dialysis membrane (Spectrum Labs™) for NFR5 constructs while VHHs were dialyzed using a Spectra/Por® 3.5 kDa MWCO dialysis membrane (Spectrum Labs™). For NFR5 constructs, Histidine-tagged TEV protease was added into the dialysis bag in a 1:50 molar ratio. NFR5 was separated from TEV protease and cleaved fusion-tag by a second Ni-AC purification step using dialysis buffer and Buffer B (FIGS. 2C-2D). Successful cleavage was evident from the complete band shift between the non-digested and digested samples, as analyzed by SDS-PAGE (FIG. 2D). Size exclusion chromatography (SEC) was performed as a final purification step on an ÄKTA™ Pure FPLC system using a HiLoad® 16/600 Superdex® 75 pg or Superdex® 75 increase 10/300 GL column (Cytiva) in SEC buffer (50 mM Tris-HCl pH 8, 100 mM NαCl, 5 mM β-mercaptocthanol) (FIGS. 2E-2F). A highly pure final preparation was obtained. The chromatogram revealed that NFR5 eluted as a monodisperse peak with a Ve of 67.83 ml, corresponding to a molecular size of ˜41 kDa and deviating only slightly from the theoretical molecular weight of 35.6 kDa. All purification steps were analyzed by SDS-PAGE (FIGS. 2B, 2D, and 2F) and relevant fractions were pooled to a final purity of >95% estimated by Coomassie® staining.

### Polyacrylamide Gel Electrophoresis Assays

Polyacrylamide gel electrophoresis (PAGE) was performed using self-cast gels and run on a Mini-PROTEAN® Tetra Cell system (Bio-Rad). Gels were cast using 12% acrylamide, Tris-HCl pH 8.8 buffered solutions supplemented with 0.1% sodium dodecyl sulfate (SDS) for denaturing PAGE or without SDS for native PAGE. All gels were cast as discontinuous gels with a Tris-HCl pH 6.8, 5% acrylamide stacking gel. A 25 mM Tris-HCl, 192 mM glycine pH 8.3 running buffer was used and supplemented with 1% w/v SDS for denaturing PAGE. Denaturing SDS-PAGE was performed at room temperature. Native PAGE was performed at 4° C. Gels were subsequently stained with Coomassic® brilliant blue.

### VHH Selection by Phage Display

One Lama glama was immunized with 400 μg NFR5 (residues 276-595; SEQ ID NO: 146) and a blood sample retrieved after 8 weeks. Peripheral blood lymphocytes were isolated by Ficoll® gradient centrifugation and total RNA was purified using an RNeasy® Plus Mini Kit (QIAGEN). cDNA was amplified using random hexamer primers and a SuperScript™ III First-Strand Synthesis System (Invitrogen™). The VHH regions were amplified by PCR and cloned into phagemid vectors as E-tag pIII fusions. E. coli ER2738 were transformed with VHH-inserted phagemid and M13 phage-display libraries were generated using VCSM13 helper phages. Nunc-Immuno™ MicroWell™ 96 well MaxiSorp™ Microplates (ThermoScientific) were coated with 1 μg NFR5 overnight at 4° C. and blocked by addition of phosphate buffered saline (PBS), 2% w/v BSA for 1 hour at room temperature. 3x 1012 phages per selection were blocked in PBS, 2% w/v BSA for 1 hour before applying to NFR5-coated wells for an additional 1 hour. Wells were washed 15 times in PBS-T (PBS supplemented with 0.1% Tween® 20) followed by 15 washes in PBS. Phages were eluted by addition of 100 μL 0.2 M glycine pH 2.2 and incubated on a shaking table at room temperature for 10 minutes. Elutions were pH adjusted by addition of 15 μL 1 M Tris-HCl pH 9.1 and used for infecting 0.8 mL ER2738 OD₆₀₀=0.6. The phage-library was amplified in ER2738 and used in a second round of selection against 0.1 μg NFR5. After the second selection round, infected ER2738 were plated on LA, supplemented with 2% w/v glucose, 100 μg/mL AMP and 10 μg/mL tetracycline (TET) and grown overnight at 37° C. Single colonies were picked and inoculated into a 96-well micro-titre plate filled with LB supplemented with 100 μg/mL AMP and 10 μg/mL TET. The E. coli 96-well micro-titre plate was covered with an AirPore Tape Sheet (QIAGEN) and incubated for 6 hours at 37° C., 95 RPM at high humidity before supplementing with 0.8 mM IPTG. E-tagged VHHs were expressed overnight at 30° C., 95 RPM in a shaking incubator. VHHs were screened for binding in an enzyme-linked immunosorbent assay (ELISA) by coating Nunc-Immuno™ MicroWell™ 96 well MaxiSorp™ Microplates (ThermoScientific) with 0.1 μg per well NFR5 overnight at 4° C. Wells were blocked the following day in PBS with 2% w/v BSA for 1 hour at room temperature; control plates only coated with PBS with 2% w/v BSA were included in parallel. The E. coli 96-well micro-titre plate was centrifuged at 1000 g for 15 minutes and 50 μL supernatant containing expressed E-tagged VHHs was transferred to the coated ELISA plates. E-tagged VHHs were allowed to bind NFR5 for 1 hour at room temperature and the ELISA wells were subsequently washed 6 times in PBS-T. The secondary anti-E-tag antibody coupled to horseradish peroxidase (Bethyl Laboratories®) was diluted 10000-fold in PBS with 2% w/v BSA, and 100 μL was added to each ELISA well and incubated for 1 hour at room temperature. Wells were washed 3 times in PBS-T and the ELISA plate was developed by addition of 100 μL 3,3′,5,5′-Tetramethylbenzidine (TMB Substrate Solution) (ThermoFisher) to each well. Reactions were quenched with 100 μL 1 M HCl and ELISA plates were read at 450 nm using a Varioskan™ LUX Multimode Microplate Reader (Thermo Scientific™). Positive clones were propagated in 5 mL LB supplemented with 100 μg/mL AMP and phagemid DNA was purified and sequenced. VHH candidates were selected based on phylogenetic alignment analysis in CLC Main Workbench 20.0.4 (QIAGEN) using neighbor-joining based on Jukes-Cantor distances.

### Analytical Size Exclusion Chromatography (SEC)

Analytical SEC was used to assay NFR5: VHH complex formation. 100 μg NFR5 was mixed in a 1:1.5 molar ratio with VHH Nb200 (SEQ ID NO: 119) and incubated on ice for 30 minutes. Assays were performed using a ÄKTA™ Pure FPLC system and a Superdex® 75 increase 10/300 (Cytiva) in SEC buffer. Samples were cleared by centrifugation at 16000×g, 4° C. for 30 minutes before sample application was performed using a 100 mL capillary loop. Protein was eluted in SEC buffer and chromatograms were processed in Unicorn™ 6 (Cytiva) and analyzed in GraphPad Prism 9 (Graphstats Technologies).

### Crystallization and Structure Determination

Milligram scale NFR5: VHH complexes were isolated utilizing SEC as described above in SEC buffer. Crystallization was screened by sitting drop vapor diffusion in a 96-well format using the Index™ commercial screen (Hampton Research). 100+100 nL sample/reservoir droplets were set up at room temperature, 85% humidity using a mosquito® Xtal3 (SPT Labtech) and plates were subsequently stored at 19° C. Optimization grid screens were manually pipetted in 1+1 mL droplets using 24-well sitting drop vapor diffusion plates (Molecular Dimensions) and stored at 19° C. Crystals were optionally produced through batch or streak seeding approaches. Crystals were manually retrieved using Mounted LithoLoops™ mounted in CryoCaps™ (SPINE format, Molecular Dimensions) attached on a Magnetic Cryo Wand (Molecular Dimensions) and step soaking cryo-protected in reservoir supplemented with 20% w/v PEG-200 (final concentration). Crystals were flash cooled at −196° C. in liquid nitrogen.

X-ray diffraction experiments were performed under cryogenic conditions. Native datasets were collected at λ˜1 Å using an Eiger 16M detector (Dectris®). Diffraction data was processed in xds and xscale (Kabsch (2010) Acta Cryst D. 66, 125-132). The NFR5-Nb200 dataset was analyzed in Xtriage from the PHENIX program suite (Adams et al. (2010) Acta Cryst D. 66:213-221) and was elliptically truncated using the UCLA-DOE Diffraction Anisotropy Server (Strong et al. (2006) PNAS 103:8060-8065), due to anisotropy. The crystallographic phase problem was solved by molecular replacement in Phaser (McCoy et al. (2007) J Appl Cryst. 40:658-674), using the Arabidopsis thaliana BAK1 protein kinase structure (PDB-ID: 3UIM) as a search model (Yan et al. 2012 Cell Research 22 (8): 1304-1308). Data refinement was performed using phenix.refine (Adams et al. (2010) Acta Cryst D. 66:213-221) and the atomic model was built in Coot (Emsley et al. (2010) Acta Cryst D. 66:486-501). The model was validated using MolProbity (Williams et al. (2018) Protein Scence 27:293-315) and the wwPDB Validation Server (validate-rcsb-1[dot]wwpdb[dot]org). Data collection statistics for both ellipsoidal and spherical datasets and refinement statements are reported in Table 1. Structural analyses were performed and figures were produced using PyMOL Molecular Graphics System, version 2.4 Schrödinger, LCC.

### Phosphorylation Assay

3 μg of NFR1 or NFR5 in 10 μL SEC buffer was supplemented with 5 mM MgCl₂, 100 nCi [γ-³²P] ATP (PerkinElmer®) and incubated for 1 hour at room temperature. Phosphorylation reactions were stopped by addition of SDS loading dye and 95° C. incubation. Phosphorylation reactions were assayed by SDS PAGE and the gel was placed in a Hypercassette™ Autoradiography Cassette (Amersham™/Cytiva Life Sciences™) overnight. The radiograph was developed using a Typhoon™ FLA 9500 (Amersham™/Cytiva Life Sciences™) and the gel was stained with Coomassie® brilliant blue.

### Bio-Layer Interferometry (BLI) Assays

Streptavidin (SA) biosensors (kinetic quality, Molecular devices, FortéBio) were equilibrated in Binding buffer (50 mM Tris-HCl pH 8, 200 mM NαCl, 0.01% TWEEN® 20) before use. AviTag™ NFR5 in Binding buffer was immobilized on SA sensors until 0.1 nm using an Octet® RED96 (Molecular devices, ForteBio) at 25° C. SA biosensors with immobilized Avi-tagged NFR5 were washed for 120 seconds in Binding buffer before binding was assayed over a 1:1 v/v dilution series (3.6-0.07 mg/ml, 100-1.56 μM) of NFR5 WT, NFR5 L290E L291E (2E; SEQ ID NO: 148), and NFR5 V294E Y297E V298E (3E; SEQ ID NO: 149). Unspecific binding was measured to a biotin loaded SA sensor and was subtracted from the interaction datasets. Data was processed and analyzed in FortéBio Data Analysis 7 (Molecular devices, ForteBio) and GraphPad prism 9 (Graphstats Technologies). Assays using VHH Nb200 and the VHH Nb200 control (A51E Y60E V100E; SEQ ID NO: 151) were performed using an immobilization degree of 1 nm Avi-tagged NFR5 and binding was assayed over a 1:1 v/v dilution series (5-0.08 M). The dissociation constant (K_{d}) was calculated in GraphPad prism 9 (Graphstats Technologies) using an association/dissociation non-linear regression model.

### Results

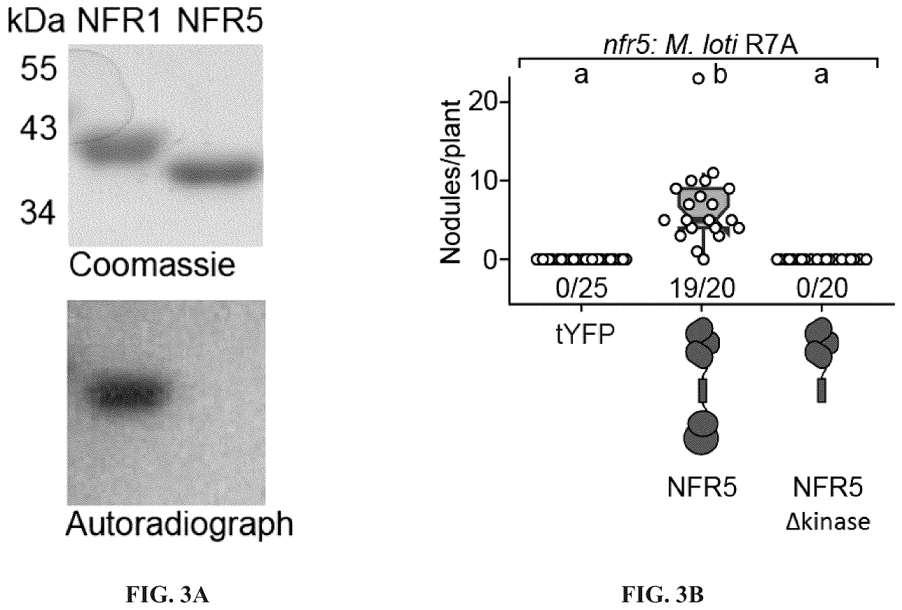

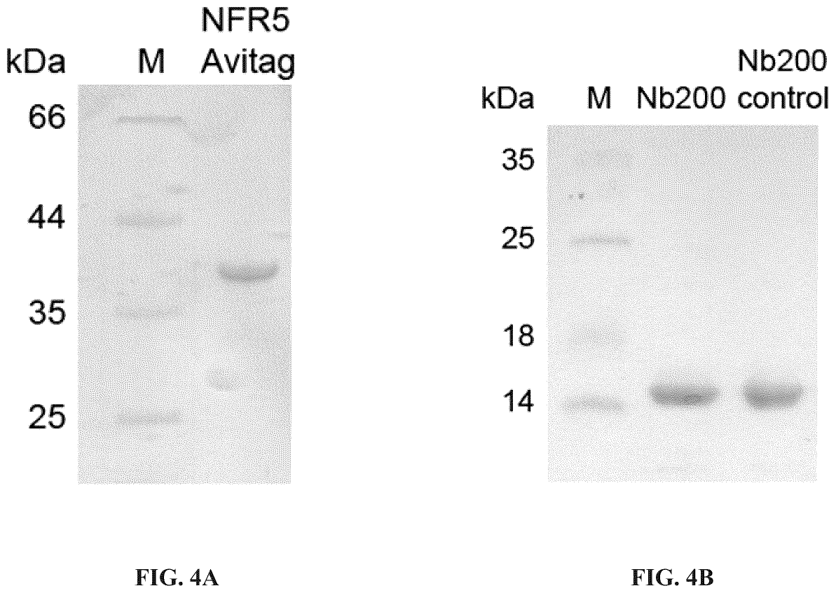

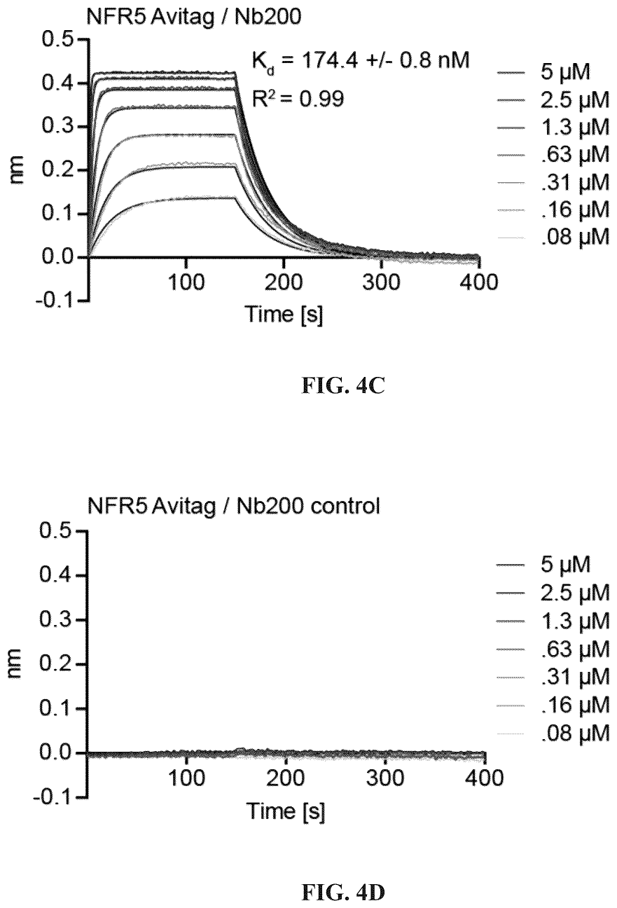

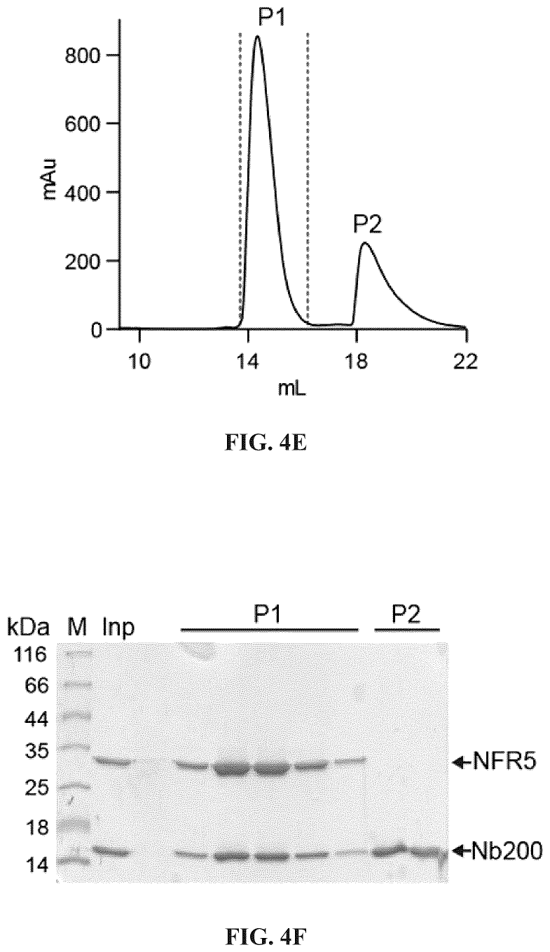

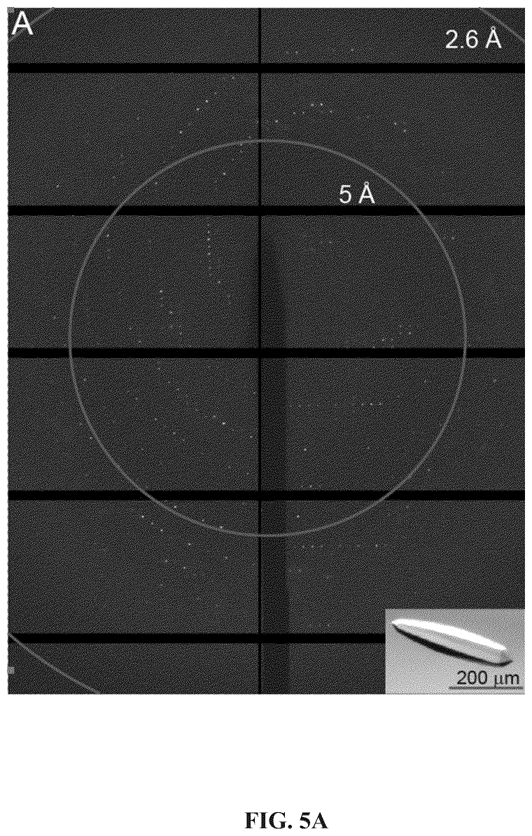

It was previously reported that NFR5 contains a catalytically inactive pseudokinase as opposed to its partner receptor NFR1 that contains a catalytically active protein kinase (Madsen et al. (2011), Plant J 65, 404-417). This observation was first validated using pure and stable recombinant protein (FIG. 1, FIGS. 2A-2F) in a radiolabeling phosphorylation assay, which confirmed that NFR5 is catalytically inactive (FIG. 3A). Despite the lack of catalytic activity, previous reports found that NFR5 signaling requires the intracellular domain (Miyata et al. (2016), Plant Cell Physiol 57, 2283-2290; Pietraszewska-Bogiel et al. (2013), PLOS One 8, e65055). The ability of an NFR5 receptor lacking the intracellular domain to drive M. loti nodulation when expressed from the Nfr5 promoter and terminator in Agrobacterium rhizogenes-induced roots was tested using Lotus nfr5 mutant plants. It was found that the NFR5 intracellular domain was essential for rhizobia symbiosis in Lotus (FIG. 3B). Next, determination of the molecular structure of the intracellular domain was sought in order to understand the signaling mechanisms of NFR5. There was a failure to produce well-diffracting crystals despite exhaustive attempts with multiple constructs of NFR5. To facilitate crystallization, a llama was immunized and a heavy chain variable domain (VHH) against NFR5 was selected using phage display. The dissociation constant of the VHH, referred to as Nb200, was measured to be in the nanomolar range for NFR5 binding, which allowed for isolation and crystallization of a stable complex between Nb200 and a C-terminal tailless NFR5 (residues 276-563 (SEQ ID NO: 147), FIGS. 4A-4F).

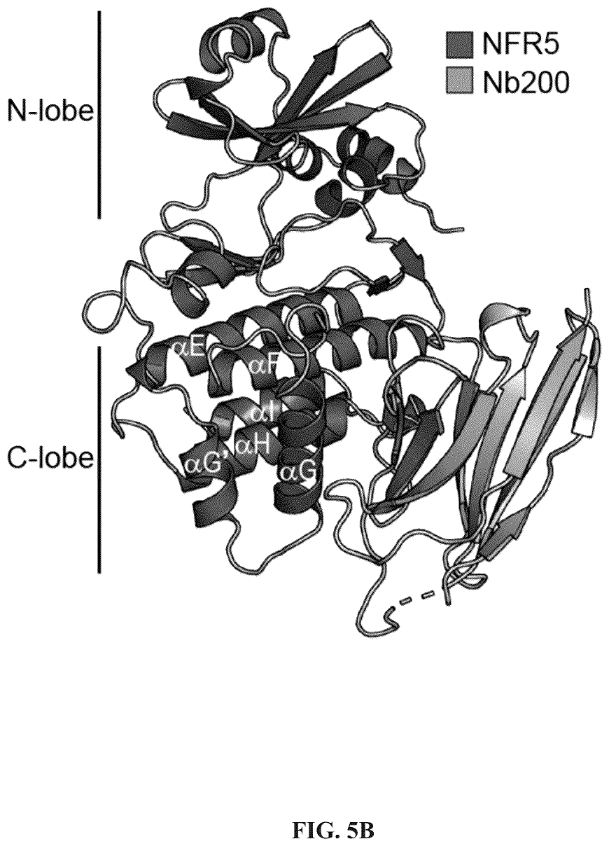

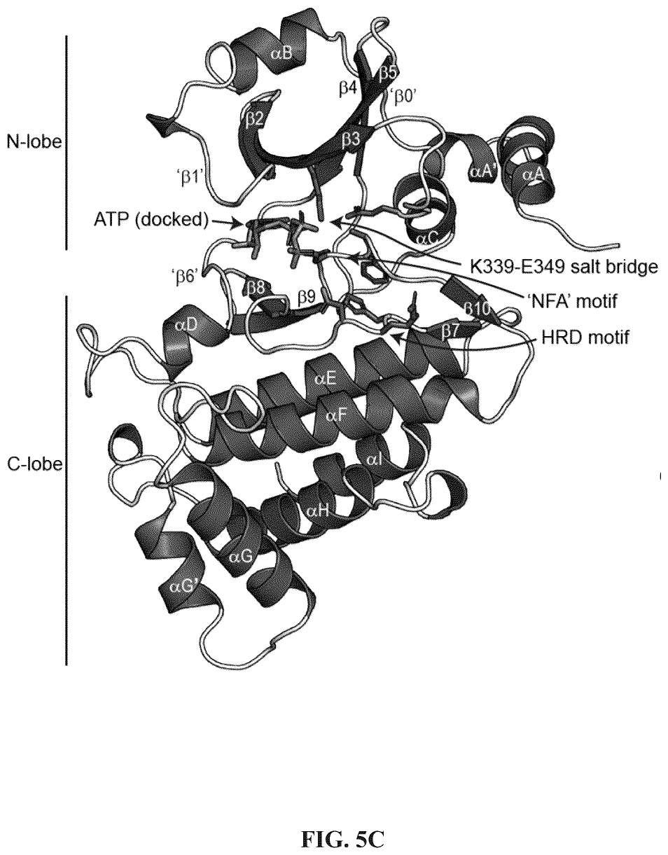

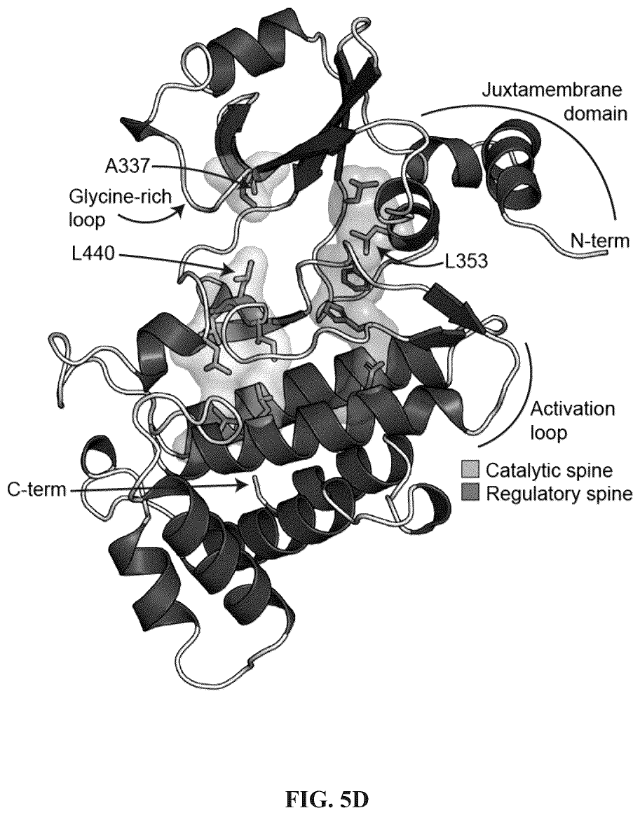

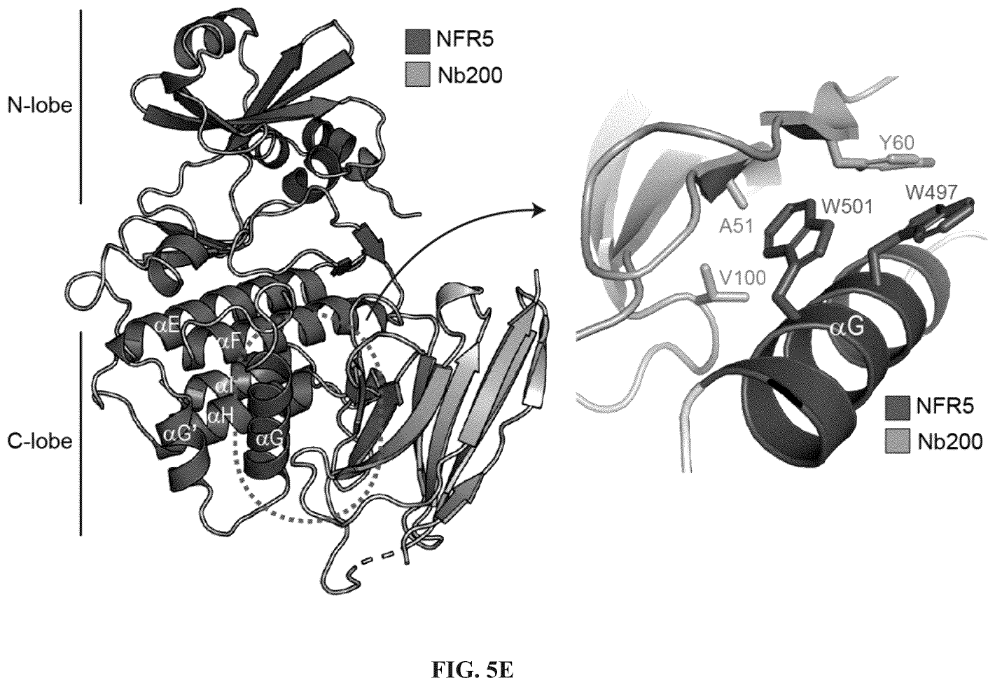

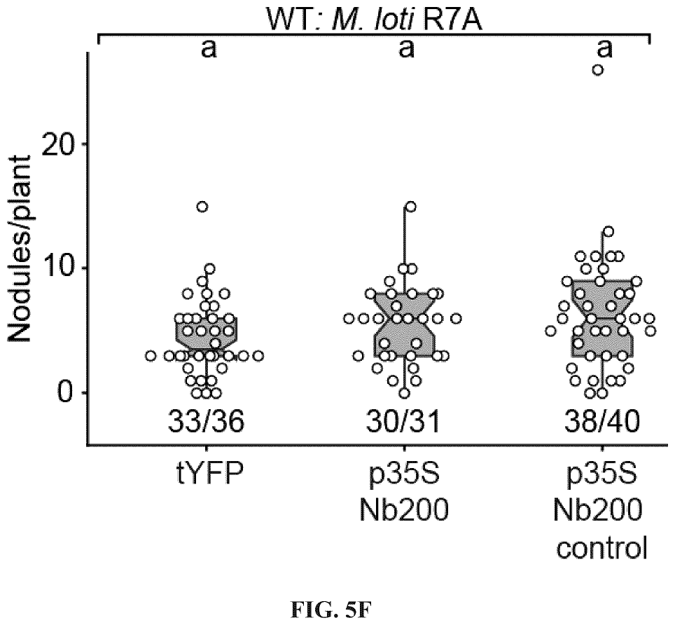

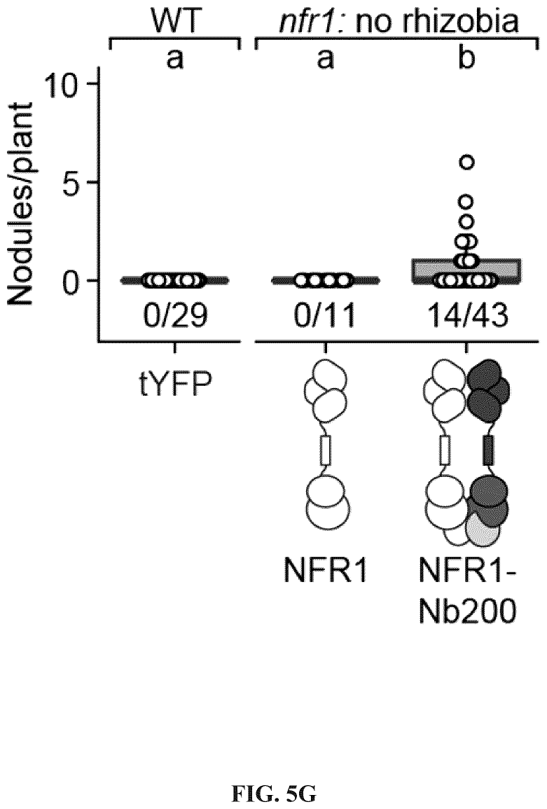

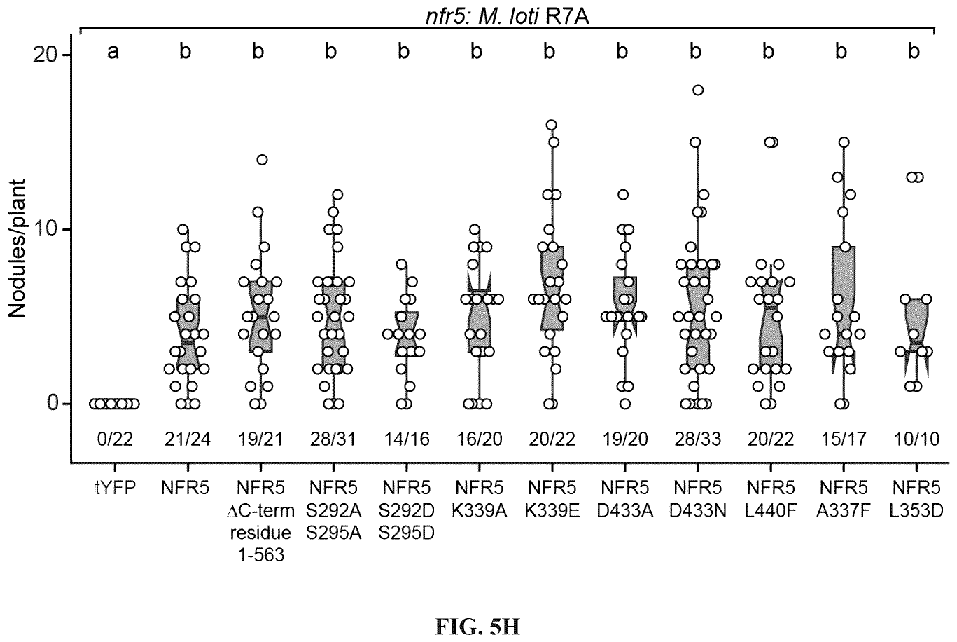

An anisotropic dataset to 2.6 Å resolution was collected and used to determine the VHH-bound structure of the NFR5 intracellular domain (FIGS. 5A-5B, Table 1).

TABLE 1

| Data collection | Ellipsoidal truncated | | Spherical truncated | |
|---|---|---|---|---|
| Beamline | BioMAX, MAX IV | | | |
| Wavelength (Å) | 0.92 | | | |
| Spacegroup | P 31 2 1 (No. 152) | | | |
| a, b, c (Å) | 126.83, 126.83, 137.94 | | | |
| α, β, γ (°) | 90, 90, 120 | | | |
| Max res. a*, b*, c* (Å) | 3.1, 3.0, 2.6 | | | |
| Resolution range (Å) | 39.75-2.66 | (2.73-2.66) | 50-2.65 | (2.72-2.65) |
| Total reflections | 285379 | (1444) | 373687 | (28508) |
| Unique reflections | 30151 | (203) | 37596 | (2728) |
| Redundancy | 9.46 | (7.11) | 9.9 | (10.5) |
| Completeness (%) | 81.1 | (7.5) | 99.6 | (99.3) |
| Mean I/s(I) | 22.45 | (6.13) | 18.42 | (1.05) |
| CC1/2 (%) | 99.9 | (94.4) | 99.9 | (65.4) |
| Rmeas (%) | 6.4 | (43.6) | 7.5 | (381) |

| Wilson B-factor (Å²) | 69.13 | |
|---|---|---|
| Refinement | | |
| Refined reflections | 30146 | (358) |
| Test set reflections | 1600 | (16) |
| Rwork (%) | 22.91 | (37.99) |
| Rfree (%) | 24.95 | (64.90) |

| | |
|---|---|
| Number of atoms | |
| Non-H in ASU | 6093 |
| Protein | 6069 |
| Ligand/Waters | 4/20 |
| R.M.S. bonds/angles | 0.003/0.60 |
| Ramachandran (%) | |
| Favoured/outlier | 96.71 |
| Allowed | 3.29 |
| Outlier | 0 |
| Rotamer outliers (%) | 2.25 |
| Clashscore | 9.03 |
| Average B-factors (Å²) | |
| Protein | 82.49 |
| Ligand/Waters | 49.81/58.6 |

Data collection and refinement statistics for the NFR5-Nb2_{NFR5}, PDB: 8S79 crystal structure. The NFR5-Nb2_{NFR5} dataset was analyzed in xtriage from the PHENIX program suite (P. D. Adams et al., PHENIX: a comprehensive Python-based system for macromolecular structure solution. Acta Crystallogr D Biol Crystallogr 66, 213-221 (2010)). Due to anisotropy the dataset was subjected to ellipsoidal truncation using the UCLA-DOE diffraction anisotropy server (Strong et al. (2006) PNAS 103(21): 8060-8065). The crystallographic phase problem was solved by molecular replacement in Phaser (A. J. McCoy et al., Phaser crystallographic software. J Appl Crystallogr 40, 658-674 (2007)), using the Arabidopsis thaliana BAK1 protein kinase structure (PDB-ID: 3UIM) as a search model (L. Yan et al., Structural basis for the impact of phosphorylation on the activation of plant receptor-like kinase BAK1. Cell Res 22, 1304-1308 (2012)). Data refinement was performed using phenix.refine and the atomic model was built in Coot (P. Emsley, B. Lohkamp, W. G. Scott, K. Cowtan, Features and development of Coot. Acta Crystallogr D Biol Crystallogr 66, 486-501 (2010)). The model was validated using MolProbity (C. J. Williams et al., MolProbity: More and better reference data for improved all-atom structure validation. Protein Sci 27, 293-315 (2018)) and the wwPDB Validation Server (validate-rcsb-1[dot]wwpdb[dot]org). Data collection statistics for both ellipsoidal and spherical datasets and refinement statistics are reported in Table 1.

### Example 2: The Determined NFR5 Intracellular Structure Represents the Complete Functional Intracellular Signaling Domain

The following example demonstrates that the determined structure of the intracellular NFR5 is complete and represents the entire functional intracellular signaling domain as determined by complementation assays.

### Materials and Methods

### Nodulation Assay

Constructs were made with the native NFR5 promoter upstream (or driving) NFR5 or mutant NFR5. Multi-level Golden Gate cloning was used to assemble constructs with a transformation marker containing a pLjUbi: tYFPnls-tNOS module in the pIV10 cloning vector. Constructs were conjugated into Agrobacterium rhizogenes AR1193 strain for hairy root transformation as described (S. Radutoiu, L. H. Madsen, E. B. Madsen, A. M. Nielsen, J. Stougaard, Agrobacterium rhizogenes pRi TL-DNA integration system: a gene vector for Lotus japonicus transformation. A. J. Márquez, Ed., Lotus japonicus Handbook (Springer, Dordrecht, The Netherlands, 2005), 10.1007/1-4020-3735-X_28). An A. rhizogenes strain containing only the transformation marker (pLjUbi:tYFPnls-tNOS) was used as negative control. Protein sequences used in constructs in this and following Examples are presented in Table 2.

TABLE 2
Protein sequences for plant experiments.

| Description | SEQ ID NO |
|---|---|
| NFR5 residues 1-595 | 8 |
| NFR5 residues 276-595 | 146 |
| NFR5 residues 276-563 | 147 |
| NFR5 residues 276-595, L290E/L291E (2E) | 148 |
| NFR5 residues 276-595, V294E Y297E | 149 |
| V298E (3E) | |
| NFR5 residues 276-595, Avitag | 150 |
| Nb200 A51E Y60E V100E (Nb2NFR5) | 151 |
| NFR1 residues 263-599 | 152 |
| Nb200 | 119 |
| NFR5 residues 1-300, Δkinase domain | 161 |
| NFR1 residues 1-623 | 162 |
| NFR1-Nb2NFR5 | 163 |
| NFR5 (L290E_L291E) | 164 |
| NFR5 (L290F_L291F) | 165 |
| NFR5 (V294E_Y297E_V298E) | 166 |
| NFR5/RLK10 chimera | 167 |
| NFR5/RLK10 (L322E_L323E) | 168 |
| NFR5/RLK10 (V326E_F329E_I330E) | 169 |
| NFR5 (S292A_S295A) | 170 |
| NFR5 (S292D_295D) | 171 |
| NFR5 (K339A) | 172 |
| NFR5 (K339E) | 173 |
| NFR5 (D433A) | 174 |
| NFR5 (D433N) | 175 |
| NFR5 (L440F) | 176 |
| NFR5 (A337F) | 177 |
| NFR5 (L353D) | 178 |

The Lotus japonicus wild-type (ecotype Gifu) and mutant line nfr5-2 were used for nodulation assays. Plants were grown at 21° C. (16/8-hour light/dark conditions).

Lotus seeds were scarified by sandpaper followed by a surface sterilization in bleach (3% chlorine) for 5 minutes, washed 5 times with water, then soaked in water and kept at 4° C. overnight. Seeds were placed on wet filter papers for 4-5 days at 21° C., under 16 hours photoperiod for germination. Germinated seedlings were transferred to square plates with ½ Gamborg's B5 nutrient (Duchefa Biochemie) solidified by 0.8% Gelrite (Duchefa Biochemie). A. rhizogenes AR1193 strains carrying the construct of interest were grown for three days on LB Agar containing Ampicillin, Rifampicin, and Spectinomycin (final concentration of each antibiotic was 100 μg/ml). For each construct the cells grown on one plate were resuspended in 4 mL YMB (5 g/L mannitol, 0.5 g/L yeast extract, 0.5 g/L K₂HPO₄, 0.2 g/L MgSO₄·7H₂O, 0.1 g NαCl, pH=6.8). The bacterial suspension was then used to transform 7-day-old seedlings using a 1 mL syringe with a needle (Sterican® Ø 0.40×20 mm), punching the hypocotyl with a syringe needling, and p dropping bacteria solution on top of the wounded area. Square plates containing the transformed seedlings were sealed and left in the dark overnight and then moved to 21° C. under 16/8-hour light/dark conditions. After three weeks, untransformed roots were removed and transformed plants were transferred to Magenta™ vessels (Sigma-Aldrich) filled with lightweight expanded clay aggregate (Leca®, 2-4 mm; Saint-Gobain Weber A/S) supplemented with 80 mL nitrogen-free ¼×B&D nutrient solution per box.

Each Magenta™ vessel was inoculated with 1 mL of Mesorhizobium loti R7A carrying DsRED marker at OD₆₀₀=0.04. Plants were harvested 6 weeks after inoculation and nodules were counted and imaged using a Leica FluoStereo M165FC microscope equipped with a Leica DFC310 FX camera.

A Kruskal-Wallis test (Kruskal et al. (1952) J Am Stat Assoc 47, 583-621) followed by Dunn's post-hoc test (Dunn (1964) Technometrics 6, 241-252) was used for statistical analysis of nodule counts.

### Results

All secondary structural elements of the kinase domain were resolved in the electron density map, including the αB helix and the tyrosine gatekeeper residue that are hallmarks of the IRAK/Pelle-type kinases (FIGS. 5C-5D) (Shiu & Bleecker (2001), Proc Natl Acad Sci USA 98, 10763-10768; Wang et al. (2006) Structure 14, 1835-1844). The generated model revealed that the VHH Nb200 contacted the αG helix, which led to speculation that Nb200 could possibly interfere with NFR5 signaling due to blocking this structural element (FIGS. 5B, 5E). The VHH Nb200 and a VHH Nb200 control incapable of NFR5 binding were overexpressed in wild-type Lotus, but no effect on nodulation efficiency was observed (FIG. 5F). Additionally, an NFR1-NFR5 complex was formed by fusing Nb200 to the C-terminus of NFR1 and spontaneous nodule formation was observed independent of M. loti inoculation, which demonstrated that Nb200 does not inhibit signaling from NFR5 (FIG. 5G) (Rübsam et al. (2023), Science 379, 272-277). Other work recently showed that the C-terminal tail of Lotus SYMRK had an essential function in root nodule symbiosis signaling (Abel et al. (2024), Proc Natl Acad Sci USA 121, c2311522121), but the NFR5 construct lacking the C-terminal tail was able to complement Lotus nfr5 and formed nodule numbers comparable to wild-type NFR5 complementation (FIG. 5H). The VHH-bound structure therefore represented the signaling competent intracellular domain of NFR5.

The degenerated glycine-rich loop is among the features that classify NFR5 as a pseudokinase. Despite this, an ATP molecule can be docked into the ATP binding pocket with no steric clashes and thereby assemble the catalytic spine (FIGS. 5C-5D). Interestingly, the regulatory spine (R-spine) of NFR5 was intact and fully formed along with the hallmark K339-E349 salt-bridge and αC “in” conformation, which mimicked an activated conformational state in conventional protein kinases (FIG. 5D). Signaling by conformational toggling of the αC, C- and R-spines was investigated by mutagenesis disruption of the catalytic site, K339-E349 salt-bridge, ATP binding pocket, and R-spine (FIG. 5F). All NFR5 variants were fully capable of driving nodulation in Lotus nfr5 comparable to wild-type NFR5 (FIG. 5H). These results suggested that conventional protein kinase motifs and conformational toggling of the αC helix, C-spine, and R-spine is unlikely to regulate NFR5 signaling.

### Example 3: Two α-Helices in the Juxtamembrane Define a Conserved Hydrophobic Motif in NFR5-Type Receptors

The following example describes the identification of a conserved juxtamembrane motif.

### Materials and Methods

NFR5 was isolated and characterized as in Examples 1 and 2.

### Sequence Alignment and Conservation Analysis

Sequences spanning the juxtamembrane to αB regions in NFR5-type receptors were aligned in CLC Main Workbench 22 (QIAGEN) using high gap cost settings. The alignment spanning the Lotus NFR5 αA, αA′, β0, and αB was used in structural conservation analysis and generation of a sequence logo. Lotus NFR5 uniprot: Q70KR1, Medicago NFP uniprot: Q0GXS4, Pea SYM10 uniprot: Q70KR3, Bean NFR5 uniprot: V7CHW9, Soybean NFR5a uniprot: A5YJV9, Chickpea NFP uniprot: A0A1S3EF43, Lupin NFR5 uniprot: A0A1J7GXGO, Peanut NFR5 NCBI: XP_025698788.1, Fragaria NFR5 NCBI: XP_004300586, Apple NFP uniprot: A0A498KGG5, Poplar NFR5 uniprot: A0A2K2AG15, Parasponia NFP2 genbank: PON37437.1, Datisca NFR5 gene: Datgl376S09111, Rice MYR0 gene: Os03g13080, Barley RLK10 NCBI: XP_044979240.1, Maize NFP NCBI: XP_020399958.1.

### Results

Two NFR5 molecules constituted the asymmetrical unit of the crystal (FIG. 6A). One molecule (chain A) is better resolved in the electron density map, and not only the core kinase domain but also the entire juxtamembrane connecting the kinase to the transmembrane helix of the receptor were able to be modelled (FIGS. 6B, 6D, 6E). The juxtamembrane appeared to be a separate structural part distinct from the kinase core with only a few intramolecular contacts to the αC helix. Unexpectedly, the juxtamembrane region formed two clearly resolved α-helices (FIG. 6B-6C). Based on the IRAK/Pelle-type kinase nomenclature these were termed α-helices αA and αA′. The αA and αA′ helices were connected by a small serine-glycine linker that bent the two helices by approximately 65° relative to each other. Notably, αA and αA′ each contained a cluster of exposed hydrophobic residues: αA L290, L291 and αA′ V294, Y297, V298 (FIG. 6F). Surface mapping the electrostatic potential of the juxtamembrane revealed these residues formed a spatially continuous hydrophobic surface (FIG. 6G). Alignment of NFR5-type receptors from different plant species revealed a striking conservation in sequence and hydrophobicity in this area, suggesting a functional role for this region, which was termed the “juxtamembrane motif” (FIGS. 6H-6J).

### Example 3: The Juxtamembrane Motif is Important for NFR5 Self-Interaction

The following example describes the determination that the juxtamembrane motif is important for NFR5 self-interaction.

### Materials and Methods

### Polyacrylamide Gel Electrophoresis Assays

NFR5 (residues 276-595; SEQ ID NO: 146) and NFR5 (residues 276-563; SEQ ID NO: 147) were expressed and purified as in Example 1.

Sodium dodecyl sulfate polyacrylamide gel electrophoresis (SDS-PAGE) was performed as in Example 1. Native PAGE was performed likewise, with the following modification. Gels were cast using Tris-HCl pH 8.8 buffered solutions without 0.1% sodium dodecyl sulfate (SDS) for native PAGE. A 25 mM Tris-HCl, 192 mM glycine pH 8.3 running buffer without SDS was used for native PAGE. Native PAGE was performed at 4° C.

### Dynamic Light Scattering Assay

A concentration series (1, 2, 3, 4, 5, 6 mg/mL) of NFR5 constructs in SEC buffer were loaded into Prometheus NT.48 Series nanoDSF Grade Standard Capillaries (NanoTemper Technologies). Particle diffusion was measured in a dynamic light scattering experiment using a NanoTemper Prometheus (NanoTemper Technologies). Samples were assayed in technical triplicates with 10 measurements performed for each capillary in High Sensitivity mode. Polydispersity indices, diffusion coefficients and particle radii were calculated in Panta Analysis (NanoTemper Technologies) and plotted using GraphPad prism 9 (Graphstats Technologies). Diffusion coefficients/concentration plots were used to derive self-interaction parameters, kSI (also known as the diffusion interaction parameter kD), by linear regression in GraphPad prism 9 (Graphstats Technologies).

### Results

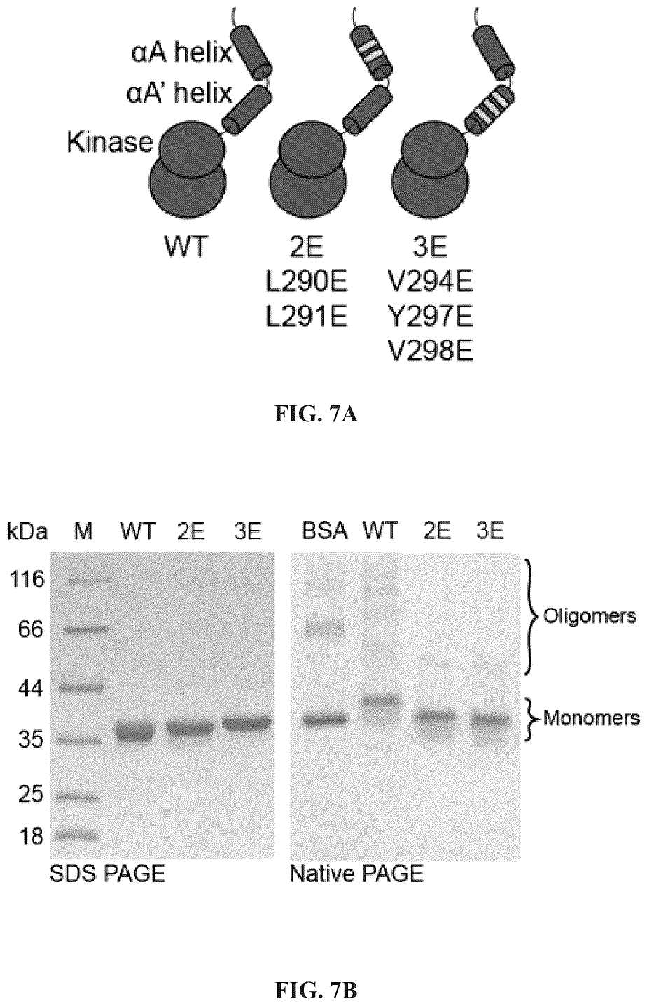

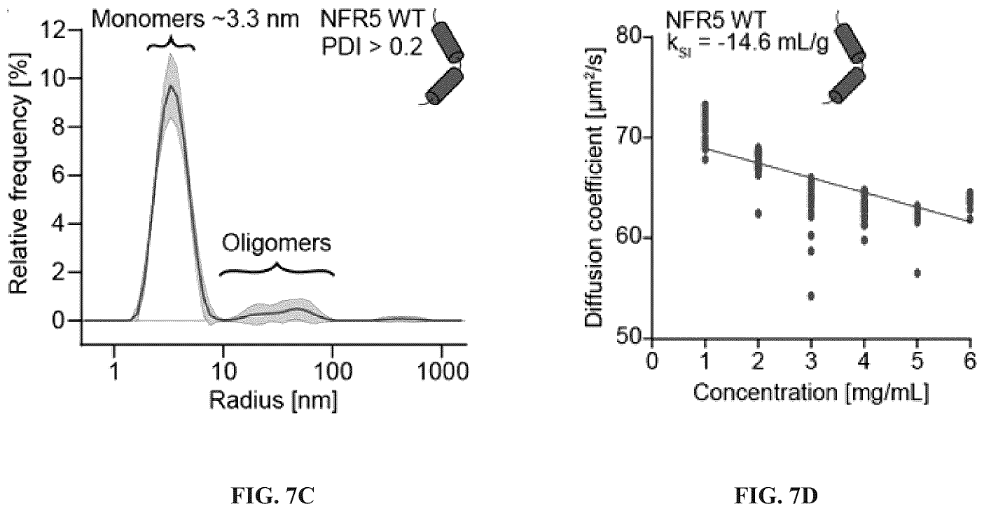

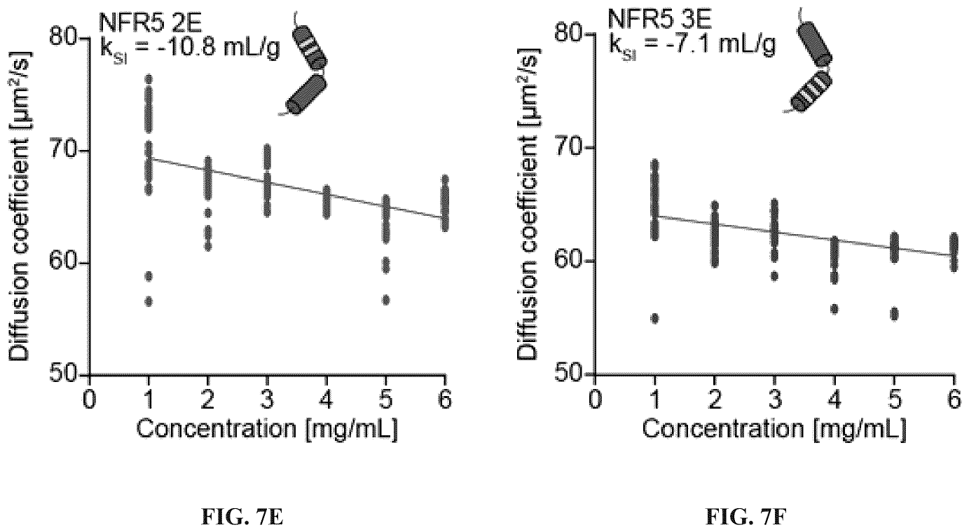

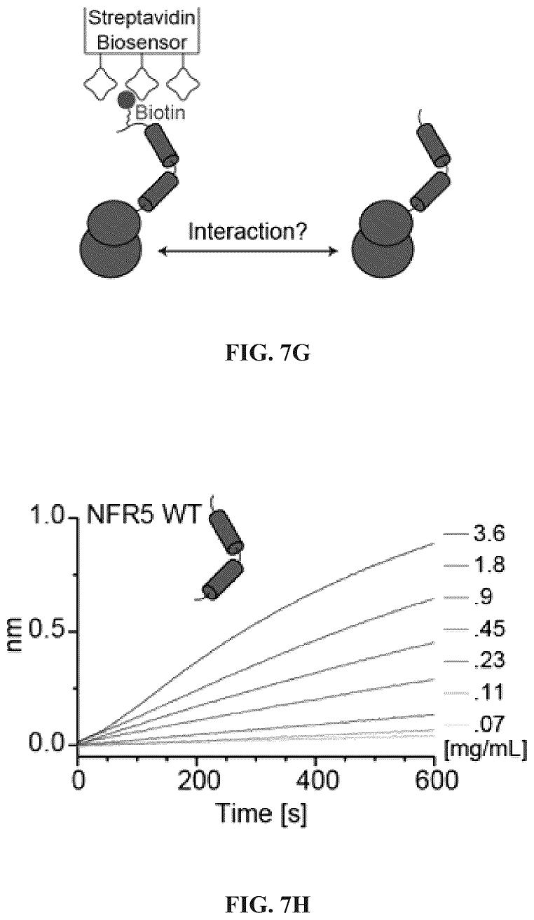

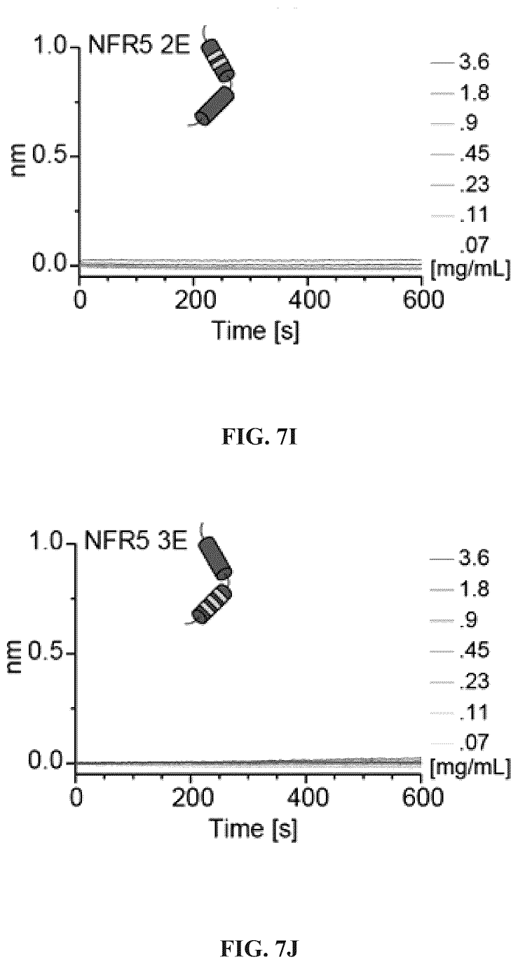

It was hypothesized that the exposed juxtamembrane motif could function as a hydrophobic interaction surface. Purified NFR5 (residues 276-595; SEQ ID NO: 146) migrated to the expected molecular mass in a denaturing SDS-PAGE experiment but showed a characteristic oligomeric migration pattern when assayed in parallel with native PAGE (FIGS. 7A-7B). The NFR5 pattern resembled the pattern formed by the well-known oligomer forms of Bovine Serum Albumin (FIG. 7B). Dynamic light scattering (DLS) was used to measure the in solution particle size dispersity of the NFR5 sample. A polydispersity index>0.2 was consistently obtained, which showed that populations of different particle sizes were present in the sample (FIG. 7C). Together this showed that the NFR5 intracellular domain self-interacted in vitro. To investigate whether this self-interaction was mediated by the juxtamembrane motif, two variants were generated with substitutions in either αA (L290E, L291E) or αA′ (V294E, Y297E, V298E) that were referred to as 2E (SEQ ID NO: 148) and 3E (SEQ ID NO: 149), respectively (FIG. 7A). It was hypothesized that the introduction of charged glutamates would abolish hydrophobic interactions engaged by the motif. By comparison, 2E and 3E appeared to be monomeric with a minor dimeric population and did not show the characteristic pattern observed for NFR5 WT in native PAGE (FIG. 7B). A second DLS experiment was performed to compare the in solution self-interaction of wildtype NFR5 with 2E and 3E by measuring their self-interaction parameter (k_{SI}) over a protein concentration series (FIGS. 7D-7F). All samples showed concentration dependent self-interaction (negative k_{SI} values), although WT displayed a higher degree of self-interaction compared to 2E and 3E. Next an N-terminal Avi-tagged NFR5 was purified in order to immobilize the NFR5 intracellular domain on a streptavidin biosensor (FIGS. 4A-4D, 7G). The immobilized NFR5 was used in a bio-layer interferometry experiment to measure interaction to WT, 2E, and 3E variants (FIGS. 7H-7J). A clear concentration dependent interaction was observed for WT, while no interaction was observed for 2E and 3E. In summary, the in vitro data showed the NFR5 intracellular domain self-interacted and that the juxtamembrane motif was important for this interaction.

It is noteworthy that the human Epidermal Growth Factor Receptor constitutively homodimerized through a juxtamembrane α-helix that contains a hydrophobic motif with striking resemblance to the NFR5 juxtamembrane motif (Endres et al. (2013), Cell 152, 543-556).

### Example 4: The Juxtamembrane-Motif in NFR5-Type Receptors is Required for Symbiosis Signaling

The following example describes identification of the role of the juxtamembrane motif of NFR5-type receptors in signaling.

### Materials and Methods

### Nodulation Assay

Nodulation assays were performed as in Example 2, with the following modifications. Mesorhizobium loti (M. loti) strain R7A constitutively expressing the fluorescent protein DsRed was grown in TY/YMB media at 28° C. After transfer to Magenta™ vessels, plants were inoculated with 150 μl per plant of M. loti R7A DsRed strain, at a final concentration of OD₆₀₀=0.04. At six weeks post inoculation, nodules were counted and pictures were acquired with a Leica M165FC Fluorescent Stereo Microscope equipped with the Leica DFC310 FX camera.

### Protein Subcellular Localization Assay

Expression of constructs were under the control of the pLjUbi promoter. Lotus japonicus protoplasts were isolated from roots of 4 days old Gifu wild-type seedlings as previously described (M. Frank et al., Single-cell analysis identifies genes facilitating rhizobium infection in Lotus japonicus. Nat Commun 14, 7171 (2023)). The viable protoplasts were resuspended in a freshly prepared 40% PEG4000 solution and 40 μg of the plasmid of interest was added. The mixture was incubated for 15 minutes at room temperature in dark. The reaction was stopped by adding 10 mL of the W5 buffer. With a gentle stir, the mixture was transferred to the centrifuge, spun down and the pellet was resuspended in 1 mL of W5 buffer. Protoplasts were imaged after 16 hours of incubation in the dark at room temperature with a LSM 980 confocal microscope.

### Results

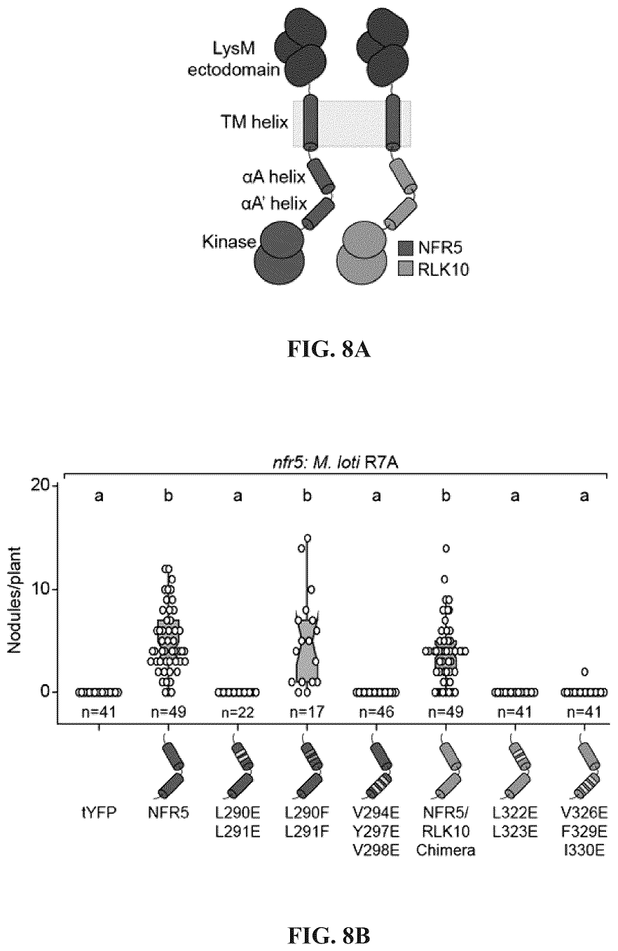

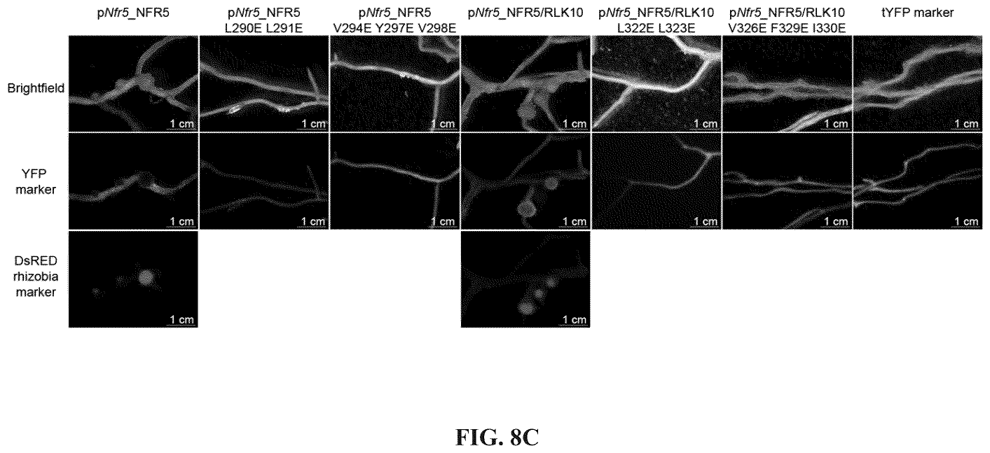

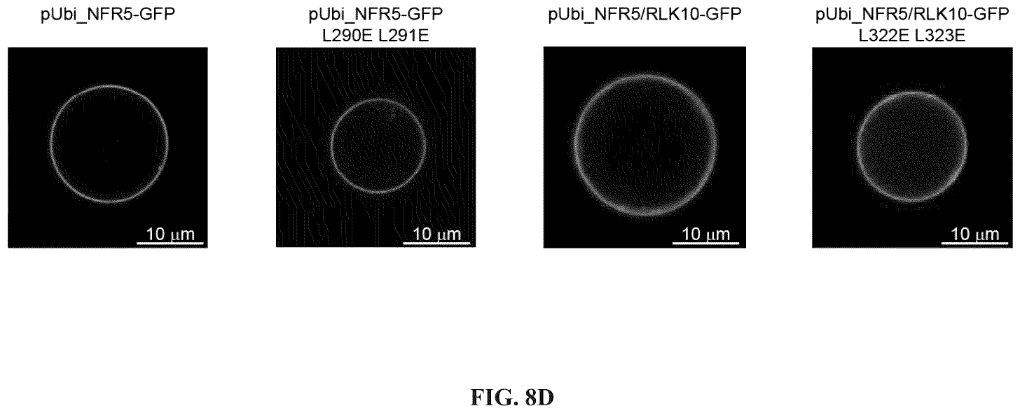

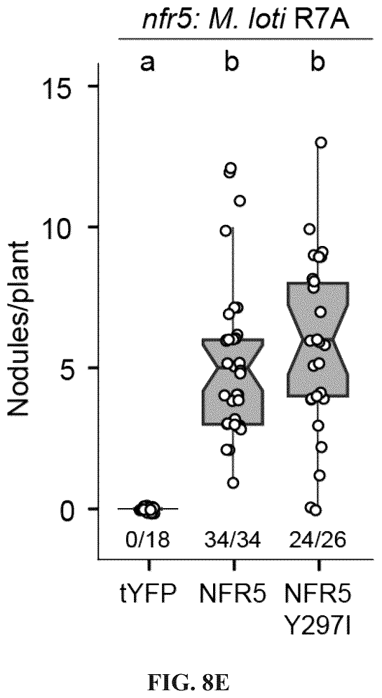

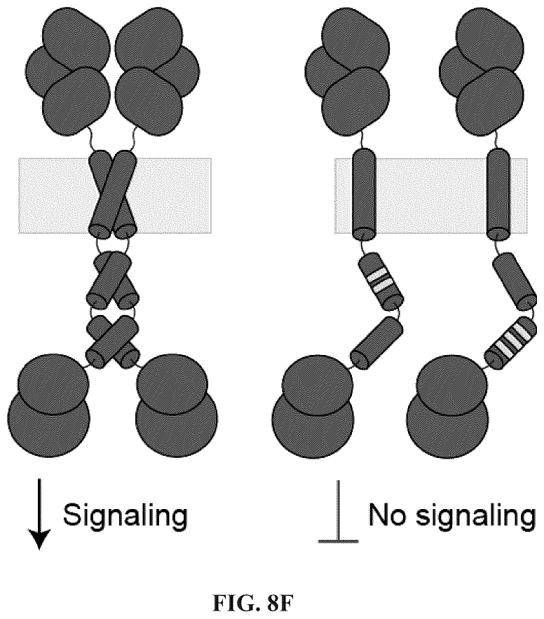

To understand whether the juxtamembrane motif was functionally important for symbiosis signaling, nodulation complementation experiments were performed in Lotus nfr5 mutant plants inoculated with M. loti. Nodulation capability of NFR5 WT, 2E (SEQ ID NO: 148), and 3E (SEQ ID NO: 149) was assayed on Agrobacterium rhizogenes induced hairy roots, using the native promoter and terminator of Nfr5 (FIGS. 8A-8B). As expected, no nodules were observed on roots transformed with a vector containing only a triple YFP transformation marker (negative control), while roots transformed with NFR5 WT formed nodules on almost all 49 plants tested (FIGS. 8B-8C). Interestingly, both 2E and 3E failed to form any nodules in a total of 68 plants (FIG. 8B). The expression and cellular localization of these receptor constructs were further investigated and determined to all localize as expected to the plasma membrane in transformed Lotus root protoplasts and Nicotiana benthamiana leaf epidermis cells (FIG. 8D). As an additional control experiment, NFR5-L290F/L291F and NFR5-Y297I were generated to test whether the specific residues or simply maintaining the hydrophobicity of the motif was important for signaling. Both these variants complemented to wild-type levels suggesting the hydrophobicity of the motifs was the essential feature for signaling (FIGS. 8B, 8E). Given the juxtamembrane motif is conserved throughout NFR5-type receptors (FIG. 6H), it was hypothesized that the motif of the barley NFR5-type receptor RLK10 could be important for signaling as well. Previous work showed that RLK10 does not complement the nodulation phenotype in Lotus nfr5 (Rübsam et al. (2023) Science 379, 272-277). This could have been due to the inability of RLK10 to perceive the specific M. loti nod factor required to initiate symbiosis signaling. Therefore, a chimeric receptor was created composed of the NFR5 extracellular and transmembrane segments coupled to the intracellular domain of RLK10 (FIG. 8A). This chimeric receptor was able to complement Lotus nfr5 to a degree comparable with wild-type NFR5, which showed that the intracellular domain of RLK10 was competent in symbiosis signaling (FIG. 8B). A 2E and 3E variant of this NFR5-RLK10 chimera were then generated, substituting the RLK10 residues L322E L323E and V326E F329E I330E, respectively. In a total of 82 plants, no nodules were observed for the RLK 10 2E variant and only a single nodule for the RLK10 3E variant. These data displayed the important signaling function for the juxtamembrane motif in barley RLK10 (FIGS. 8B-8D).

### Discussion

These data provide the crystal structure of the Lotus NFR5 intracellular domain, a result facilitated by utilizing VHH Nb200 as a crystallization chaperone. Through NFR5 mutant complementation, VHH-mediated NFR1-NFR5 complex formation, and VHH Nb200 overexpression, it was shown that the structural model represented a signaling competent NFR5. The structure revealed two structural elements in the NFR5 juxtamembrane region, αA and αA′, that contain an exposed hydrophobic surface, which may be analogous to the human Epidermal Growth Factor Receptor juxtamembrane α-helix. Self-interaction of the NFR5 intracellular domain was observed, and it was determined that the juxtamembrane motif had an important contribution in mediating this self-interaction. It is therefore likely that the phenotypic effects in the 2E and 3E variants were due to the disruption of a NFR5-NFR5 complex required for symbiosis signaling (FIG. 8F). However, these findings do not exclude a broader scaffolding function for NFR5 and the juxtamembrane motif in mediating protein-protein interactions to other important symbiosis signaling components.

In the crystal structure, the juxtamembrane motif formed a spatially continuous surface due to the bend in the linker between the αA and αA′ helices, bringing the hydrophobic residues together. In addition to this, the linker bend positioned the sidechains of the conserved NFR1 phosphorylation sites S292 and S295 into close proximity (FIG. 6F). Reduced nodulation efficiency was previously reported for single phosphorylation ablation mutants at these positions in a nfr5 complementation experiment (Madsen et al. (2011), Plant J 65, 404-417). The complementation capacity of the double ablation (S292A, S295A) and the double phosphorylation mimic (S292D, S295D) mutants was tested, but no reduction was observed in nodulation compared to wild-type NFR5 (FIG. 5H). This suggested the signaling function of the juxtamembrane motif was not regulated by phosphorylation. This result however indicates a dynamic nature of the juxtamembrane segment given that αA and αA′ could not adopt the conformation observed in the crystal structure in S292D, S295D due to electrostatic repulsion. Furthermore, the juxtamembrane region was not resolved in the electron density map of the second asymmetrical molecule of NFR5 (chain B), corroborating that the αA and αA′ helices are dynamic. A scenario where a single α-helix spans the αA and αA′ regions would however accommodate S292, S295D without repulsion and the spatial continuity of the juxtamembrane motif. It is therefore possible that NFR5-type receptors could adopt a single juxtamembrane α-helix that maintains a continuous hydrophobic surface and future studies can hopefully reveal how this could implicate NFR5 function.

It was previously shown that using heavy chain variable regions to bring RLK10 into an artificial complex with Lotus NFR1 or the NFR1-type barley receptor RLK4 was sufficient for inducing nodule organogenesis in Lotus (Rübsam et al. (2023) Science 379, 272-277). The present data built upon a dissection of the RLK10 receptor as a promising target and model for nitrogen-fixation engineering in cercals. The present data showed that equipping RLK10 with the extracellular domain of NFR5 is sufficient for nodulation in Lotus (FIG. 8B). This validated an carlier observation that RLK10 was fully capable of symbiosis signaling in Lotus (Rübsam et al. (2023) Science 379, 272-277). Importantly, the finding that the juxtamembrane motif is conserved and functional in non-legume receptors supports a more ancient function for this interaction motif in the common symbiotic signaling pathway, which could be important for arbuscular mycorrhizal symbiosis as well.

1. A modified plant LysM receptor polypeptide comprising:
(i) a juxtamembrane domain comprising a first αA motif, wherein the first αA motif has been modified as compared to a second αA motif of an unmodified plant LysM receptor polypeptide by insertion, deletion, or substitution of one or more amino acids, two or more amino acids, three or more amino acids, four or more amino acids, five or more amino acids, six or more amino acids, all seven amino acids, or wherein the juxtamembrane domain lacks the first αA motif and the second αA motif is inserted into the corresponding position in the juxtamembrane domain; and/or
(ii) a juxtamembrane domain comprising a first αA′ motif, wherein the first αA′ motif has been modified as compared to a second αA′ motif of an unmodified plant LysM receptor polypeptide by insertion, deletion, or substitution of one or more amino acids, two or more amino acids, three or more amino acids, four or more amino acids, five or more amino acids, six or more amino acids, all seven amino acids, or wherein the juxtamembrane domain lacks the first αA′ motif and the second αA′ motif is inserted into the corresponding position in the juxtamembrane domain.

2. The modified plant LysM receptor polypeptide of claim 1, wherein the first αA motif, the second αA motif, or both are selected from a polypeptide with 70% identity, 80% identity, 90% identity, 95% identity, or 99% identity to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 110, or SEQ ID NO: 159, optionally wherein the first αA motif, the second αA motif, or both are selected, comprise, or correspond to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 110, or corresponds to amino acids T286-S292 when aligned to SEQ ID NO: 8 (SEQ ID NO: 159).

3. The modified plant LysM receptor polypeptide of claim 1, wherein the first αA motif, the second αA motif, or both are selected, comprise, or correspond to the αA motif consensus sequence comprising X₁X₂X₃X₄LLX₅, where X₁ is T, I, G, or K; X₂ is A, Q, P, or G; X₃ is D or G; X₄ is K or E; X₅ is S, T, or P.

4. The modified plant LysM receptor polypeptide of claim 1, wherein the modification comprises disruption of the first αA motif, wherein disruption of the first αA motif includes removing, replacing, or substituting one or more residues, two or more residues, three or more residues, four or more residues, five or more residues, six or more residues, or all seven residues within the first αA motif.

5. (canceled)

6. The modified plant LysM receptor polypeptide of claim 1, wherein the first αA′ motif, the second αA′ motif, or both are selected from a polypeptide with 70% identity, 80% identity, 90% identity, 95% identity, or 99% identity to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 111, or SEQ ID NO: 160, optionally wherein the first αA motif, the second αA motif, or both are selected, comprise, or correspond to SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 111, or corresponds to amino acids G293-S299 when aligned to SEQ ID NO: 8 (SEQ ID NO: 160).

7. The modified plant LysM receptor polypeptide of claim 1, wherein the first αA′ motif, the second αA′ motif, or both are selected, comprise, or correspond to the αA′ motif consensus sequence comprising X₆VSX₇X₈X₉X₁₀, where X₆ is G or S; X₇ is G, S, E, D, or Q; X₈ is Y or F; X₉ is V, L, or I; X₁₀ is S, G, or D.

8. The modified plant LysM receptor polypeptide of claim 1, wherein the modification comprises disruption of the first αA′ motif, wherein disruption of the first αA′ motif includes removing, replacing, or substituting one or more residues, two or more residues, three or more residues, four or more residues, five or more residues, six or more residues, or all seven residues within the first αA′ motif.

9. (canceled)

10. (canceled)

11. (canceled)

12. (canceled)

13. (canceled)

14. (canceled)

15. The modified plant LysM receptor polypeptide of claim 1, wherein the modified plant LysM receptor polypeptide is a plant NFR5 LysM receptor polypeptide, optionally wherein the NFR5 receptor polypeptide is selected from the group of a polypeptide with 70% identity, 80% identity, 90% identity, 95% identity, or 99% identity to, or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23.

16. The modified plant LysM receptor polypeptide of claim 15, wherein the modified plant NFR5 LysM receptor polypeptide has reduced NFR5 nodulation signaling, as compared to the unmodified plant NFR5 LysM receptor polypeptide.

17. (canceled)

18. A genetically modified plant or part thereof comprising the modified plant LysM receptor polypeptide of claim 1, optionally wherein the plant is selected from the group consisting of cassava, yam, sweet potato, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, pigeon pea, lentil, Bambara groundnut, lupin, pulses, Medicago spp., Lotus spp., forage legumes, indigo, legume trees, and hemp.

19. A method of making the genetically modified plant or part thereof of claim 18, comprising introducing a genetic alteration to the plant or part thereof comprising a nucleic acid sequence encoding a heterologous plant LysM receptor polypeptide comprising a juxtamembrane domain comprising a modified αA motif and/or a modified αA′ motif, wherein the nucleic acid sequence is operably linked to a promoter.

20. The method of claim 19, wherein the promoter is a root specific promoter, an inducible promoter, a constitutive promoter, or a combination thereof; wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 24), a Lotus japonicus NFR1 promoter (SEQ ID NO: 124), a Lotus japonicus CERK6 promoter (SEQ ID NO: 26), a Medicago truncatula NFP promoter (SEQ ID NO: 25), a Medicago truncatula LYK3 promoter (SEQ ID NO: 27), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter; wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter; or wherein the nucleic acid sequence is inserted into the genome of the plant so that the nucleic acid sequence is operably linked to an endogenous promoter, optionally wherein the endogenous promoter is a root specific promoter.

21. A method of making the genetically modified plant or part thereof of claim 19, comprising genetically modifying the plant or part thereof by transforming the plant or part thereof with one or more gene editing components that target an endogenous nuclear genome sequence encoding an endogenous plant LysM receptor polypeptide comprising a juxtamembrane domain comprising an αA motif, wherein the αA motif is genetically modified by removing, replacing, or substituting one or more residues, two or more residues, three or more residues, four or more residues, five or more residues, six or more residues, or all seven residues within the αA motif, wherein the one or more gene editing components comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

22. (canceled)

23. A method of identifying a plant LysM receptor polypeptide able to initiate a downstream symbiosis pathway, by:
(a) providing a polypeptide sequence or a polypeptide model of a plant NFR5 receptor polypeptide juxtamembrane domain, an αA motif portion thereof, and/or an αA′ motif portion thereof and a candidate plant LysM receptor polypeptide; and
(b) aligning the candidate plant LysM receptor polypeptide to the polypeptide sequence or the polypeptide model.

24. The method of claim 23, wherein the plant LysM receptor polypeptide is able to initiate a downstream symbiosis pathway if it aligns with the αA motif and/or the αA′ motif of the NFR5 polypeptide juxtamembrane domain or is structurally similar to the αA motif and/or the αA′ motif of the NFR5 polypeptide juxtamembrane domain, optionally wherein the plant NFR5 receptor αA motif comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, or SEQ ID NO: 159.

25. An expression vector, isolated DNA molecule, or recombinant nucleic acid comprising one or more nucleic acid sequences encoding a heterologous plant LysM receptor polypeptide comprising a juxtamembrane domain comprising a modified αA motif and/or a modified αA′ motif, wherein the one or more nucleic acid sequences are operably linked to at least one expression control sequence, optionally wherein the plant LysM receptor polypeptide is a NFR5 receptor polypeptide.

26. A bacterial cell or an Agrobacterium cell comprising the expression vector, isolated DNA molecule, or recombinant nucleic acid of claim 25.

27. A genetically modified plant, plant part, plant cell, or seed comprising the expression vector, isolated DNA molecule, or recombinant nucleic acid of claim 25.

28. A composition or kit comprising the expression vector, isolated DNA molecule, or recombinant nucleic acid of claim 25 with a bacterial cell or Agrobacterium cell.

29. A method of reducing NFR5 nodulation signaling comprising: introducing a genetic alteration via an expression vector, isolated DNA molecule, or recombinant nucleic acid of claim 25, wherein the cell is a plant cell.

30. A modified plant NFR5 LysM receptor polypeptide comprising a first intracellular domain comprising a first juxtamembrane domain and a first kinase domain, wherein the first intracellular domain was modified by substituting one or more amino acids in the first intracellular domain with the corresponding amino acids from a second intracellular domain comprising a second juxtamembrane domain and a second kinase domain, wherein the first juxtamembrane domain comprises a first αA motif and/or a first αA′ motif, and wherein the second juxtamembrane domain comprises a second αA motif and/or a second αA′ motif, wherein the first plant species is a legume plant species, and wherein the second plant species is a non-legume plant species; or wherein the first plant species is a non-legume plant species, and the second plant species is a legume plant species; optionally wherein the legume plant species is selected from the group of bean, soybean, pea, chickpea, cowpea, pigeon pea, Bambara groundnut, lentil, pulses, Medicago spp., Lotus spp., forage legumes, indigo, and legume trees; and/or optionally wherein the non-legume plant species is selected from the group of cassava, yam, sweet potato, corn, rice, barley, wheat, Trema spp., apple, pear, plum, apricot, peach, almond, walnut, strawberry, raspberry, blackberry, red currant, black currant, melon, cucumber, pumpkin, squash, grape, and hemp.


SEQUENCE LISTING (178 sequences)

SEQ ID NO: 1 - protein, 595 aa, Arachis hypogaea
MAFFLPSLSSSIFLAFMLFSVTSIPTQSQQVNGTDFSCPVDSPSSCGTYVTYIAKSPNFLSLSNISDIFDTSPLSIARASNIKNEGDKLVPGQVLLIPVTCGCTQNQSFANITYELRQGDVYDIVSKTTYENLTNWRAVNNSNPDLNPVLLPIGVKVLFPLFCRCPSKKQLQKGIEYMITYVWQNNDNVSSVAAKFGASPVDILSENNYGGNFTAATYLPVLIPVTKLPVLTQPEASHGRKRSIQIPVIISISLGFTLVVAVIVISMVYAYLYQRKRTLNRRDLSAGTADKLLSGVSGYVSKPTVYEANEVIKATMNLSEQCKLGGTVYKAKIEGQVLAVKKVNQVVSEELNILQKVNHGNLVKLMGVSSDSDGNHFLVYEYADNGSLDEWLFSKLSLKASLTWYQRINIALDVAMGLQYLHEHTYPRIVHRDITTSNILLDSNFKAKIGNFSMVRTTTNPMISKIDVFAFGVVLIELLTGKKAMTTKADGEVVMLWKDIRKMFEVEDEKEKEECLRRWMDPKLECLYPVDYALSLVTLAANCTADVSLSRPTMAEVVLGLSLLTQPSQAALERSLTSSALEAEVTHVATPIAAR

SEQ ID NO: 2 - protein, 594 aa, Arachis ipaensis
MAFFLPSLSSSIFLVFMFSITSIPTQSQQVNGTDFSCPVDSPSSCGTYVTYIAKSPNFLSLSNISDIFDTSPLSIARASNIKNEGDKLVPGQVLLIPVTCGCTQNQSFANITYELRQGDMYDFVSKTTYENLTNWRAVNDSNPDLNPVLLPVGVKVLFPLFCRCPSKKQLQKGIEYMITYVWQNNDNVSSVAAKFGASAVDILSENNYGGNFTAATYLPVLIPVTKLPVLTQPEPSHGRKRSIQIPVIISISLGFTLVVAVIVISMVYAYLYQRKRTLNRRDSSAGTADKLLSGVSGYVSKPTVYEANEVIKATMNLSEQCKLGGTVYKAKIEGQVLAVKKVNQVVSEELNILQKVNHGNLVKLMGVSSDSDGNHFLVYEYADNGSLDGWLFSKLSLKASLTWYQRINIALDVAMGLQYLHEHTYPRIVHRDITTSNILLDSNFKAKIGNFSMVRTTTNPMISKIDVFAFGVVLIELLTGRKAMTTKADGEVVMLWKDIRKMFEVEDEKEKEECLRRWMDPKLECLYPVDYALSLATLAANCTADVSLSRPTMAEVVLGLSLLTQPSQAALERSLTSSALEAEVTHVATPIAAR

SEQ ID NO: 3 - protein, 590 aa, Medicago truncatula
MVSSFFHTLIFFSATHILLQLPQANGKNFSCTLNSSPSCDTYVAYFANSPNFLTLTAISDIFDTSPQSIARASNIKDENMNLIHGQLLLIPITCGCNGNGNYSFANISHLIKESESYYYLSTISYQNLTNWQTVEDSNPNLNPYLLKIGTKINIPLFCRCPSNYFAKGIEYLITYVWQPNDNLTLVASKLGASPKDIITANTNNFGQNFTVAINLPVFIPVKNLPALSQSYYSSSERKRINHFSIIISIGICLGCTILISLLLLLFYVYCLRKRKACENKCVPSVEITDKLISEVSNYVSKPTVYEVGMIMKATMNLNEMCKIGKSVYKAKIDGLVLAVKNVKGHITVTEELMILQKVNHANLVKLVGVSSGYDGNHFLVYEYAENGSLYNWLLSEFCTLSWSQRLSIAVDIAIGLQYLHEHTQPCIVHRNIKSSNILLDSKFKAKIANFSVARTTKNPMITKVDVLGYGMVLMELITGKKFLSYSEHSEVNMLWKDFKCVFDTEQKREEIVRRWMDPKLGRFYNVVEALSLFTLAVNCIEEQPLLRPTMGEVVLSLSLLTQPSPTLLEVSWTYGLDVEVAEMVTPIIAR

SEQ ID NO: 4 - protein, 595 aa, Medicago truncatula
MSAFFLPSSSHALFLVLMLFFLTNISAQPLYISETNFTCPVDSPPSCETYVAYRAQSPNFLSLSNISDIFNLSPLRIAKASNIEAEDKKLIPDQLLLVPVTCGCTKNHSFANITYSIKQGDNFFILSITSYQNLTNYLEFKNFNPNLSPTLLPLDTKVSVPLFCKCPSKNQLNKGIKYLITYVWQDNDNVTLVSSKFGASQVEMLAENNHNFTASTNRSVLIPVTSLPKLDQPSSNGRKSSSQNLALIIGISLGSAFFILVLTLSLVYVYCLKMKRLNRSTSSSETADKLLSGVSGYVSKPTMYEIDAIMEGTTNLSDNCKIGESVYKANIDGRVLAVKKIKKDASEELKILQKVNHGNLVKLMGVSSDNDGNCFLVYEYAENGSLEEWLFSESSKTSNSVVSLTWSQRITIAMDVAIGLQYMHEHTYPRIIHRDITTSNILLGSNFKAKIANFGMARTSTNSMMPKIDVFAFGVVLIELLTGKKAMTTKENGEVVILWKDFWKIFDLEGNREERLRKWMDPKLESFYPIDNALSLASLAVNCTADKSLSRPTIAEIVLCLSLLNQPSSEPMLERSLTSGLDAEATHVVTSVIAR

SEQ ID NO: 5 - protein, 603 aa, Cicer arietinum
MVSSFLPSHTLFFALMFFSATHILLQLPQINGTNFSHPLNSPPSCHTYAAYHAHSPNFLTLTSISDIFDTSPLSIARASNIKDENKELIPGQLLLVPITCACSGNGNYSSANISHFIKQGESYYYLSTISFQNLTNWQTVEDSNPNLNPYLLKLGTKIVIPLLCKCPSNYQFEKGIEYVITYVWQPNDNVTLVASKFGALPHDIITANANNFGQNFTTATNLPVFIPVTNLPVISQSYYYSSERKRSNHFPIIIAIGMSLVCTLLISLSLMVCVYCWRKRKACENRCVPSVEIANKLISEISNYVSKPKVYQVDIIMEATMNLNEQCKIGESVYKGKIDGLVLAVKNVKEKVTVTEELMILQKVNHTNLVKLIGVSSGYDGNHFLVYEYAENGSLYNWLFSEFSTSSSSGVFLTWSQRLSIAVDVAMGLQYMHEHTQPRIVHRDITSSNILLNSNFKAKIANFSAARTTKDPMITKVDVFAYGVVLLELLTGKKSLSYSENGEVNMSWKDIREIFDIEEKRDERLRRWIDPLLGRFFNVVEVLSLASIAVNCTKEQPLSRPTMGEVVLSLSLLTQHSTTTLERSWNFGLDLEDTGMVTPITAR

SEQ ID NO: 6 - protein, 595 aa, Cicer arietinum
MSVFFLPSRSHVLFLALMLFLTNISAQSQHLSGTNFSCPVDSPPSCETYVTYIAQSPNFLSLTNISDLFDISPLSIARASNIDDEDKELIPGQVLLVPVTCGCTKHRSFANNTYTIKLGDSYILVSTTSYQNLTNYLEMEDSNPGLNPNLIPPFIKVVVPIFCRCPSKTQLNKGIKYLITYVWHANDNVSTVSSKFGASQVDILTENNYNQNFASAANLPVLIPVTRLPILAQPSSNGRKRSIQLPVIIGISIGSAFFVTVLTVSLVYLYCLKMKRLNRTASLSETADKLLSGVSGYVSKPTMYEMDVIMEATMNLSDQCKIGESVYKANIDGKVLAVKKTKKDASEELKILQKVNHGNLVKLMGVSSDNEGNCFLVYEYAENGSLDEWLFLESSKTSDSTVSLTWSQRIGIAVDVAVGLQYMHEHTYPRIIHRDITTSNILLDANFKAKIANFSMARTSTNPMMPKIDVFAFGVVLIELLTGKKGVTTKENGEVVIMWKDFWMIFDLEGNKEERLRKWMDPKLENFYPIDNALSLASLAVNCTADKSLSRPTIEEIVLCLNLLNQPSSEPTLERSLTFGLDVEDTQIVTSIAAR

SEQ ID NO: 7 - protein, 578 aa, Lupinus angustifolius
MLFFTNIAVQSQQINATDFSCPVDSPPSCETYVTYIAHSPNFLSVVSISNIFDTSPLSIARASNLEAEDNTLIPDQVLLVPVTCSCIGNRSFANMSYVIKLGDTYELVVETIYEHLTNWLVVADLNPGLVPTLLTVGVEVIFPLFCRCPSKNQLNKGIKNLITYVWQPNDTVSTVSSKFGASSADILSENNYGQNFTAAIHQPFLIPVTQLPHLSQPEPSNARKSSINLMLIIGISLGCTLLVAALAVLLVYVCYLKKSVLKRKASFVETADRLLTGVSGYVSKPTVYEIDAIMEATMNLSELCKIGESVYKAKMDGRILAVKKAKEDVSEELKILQKVNHGNLVKLMGVSSDNDGNFFLVYEYAENGCLDEWMFSKSTSNSMVSLTWSQRISIAVDIAMGLQYMHEHTHPRIVHRDITTSNILIDSNFKAKISNFSMARTSTNPMMPKIDVFAFGVVLLELLTGKKALTTKENGEVVMLWKDIRKVFDLEEDNKEVMLQRWMDPKLENLYPVDYALHLASLAVNCTADKPLSRPTMAEIVLTLSLLAQPPPASLERSLTSSGLDAEATQTIAPVAAR

SEQ ID NO: 8 - protein, 595 aa, Lotus japonicus
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 9 - protein, 591 aa, Lotus japonicus
MTSFFLFTNTLFLALMMFFSTTHHILAQLSHTNGTNFSCPVDSPPSCDTYVTYFAQSPNFLTLTSISDLFDTSPLSIARASNIKDENQNLVPGQLLLVPVTCACSGSNSFSNISHMIKEGESYYYLSTTSYENLTNWETVQDSNPNYNPYLLPVGIKVVIPLFCKCPSNYHLNKGIEYLITYVWHNNDNVSLVASKFGVSTQDIISENNFSHQNFTAATNFPILIPVTQLPSLSQSYSSSERKRSNHIHIIISIGISLGSTLLIALLVLVSVTCLRKRKSSENKSLLSVEIAGKKLISGVSNYVSKSILYEFRLIMEATLNLNEQCKIGESVYKAKLDGQVLAVKKVKEDVTEEVMILQKVNHLNLVKLMGVSSGHDGNHFLVYEFAENGSLHNWLFSNSSTGSRFLTWSQRISIAVDVAMGLQYMHEHTQPSIVHRDITSSNILLDSNFKAKIANFSVARTSINPMILKVDVFGYGVVLLELLSGKKSLTNNEINHIREIFDLKEKREERIRRWMDPKIESLYPIDDALSLAFLAMNCTSEKPLSRPTMGEVVLSLSLLMTQHSPTTLERSWTCGLDVDVTEMQTLIAAR

SEQ ID NO: 10 - protein, 596 aa, Abrus precatorius
MAFFFLSYHTLFLALMFFTTTHILAQLPLTNGTNFSCPVNSIPSCDTYVAYFAQSPNFLTLTSISDIFDTSPLSIARASNMKSEYEDLIPGQLLLVPVNCSCNGNLSFATISHEIRQGDSYYSLSTTSYENLTTWEKVKDLNPHLDPYQLHAGIKIVIPLFCKCSSRYHLDKGIEYLITYVWQQNDNVTLVAAKFGASPVDIITENNISQNFNVATNLPVFIPVTQLPALSQIYSPHERKGSNHLSIVIISIGVSIVLFTLLLLVYVYCLRKKKTSSENRSGPSVETADKLISGVSCYVSKPTMYEVGVIMEATMNLNEQCRIGESMYMAKMDGQVLAVKKVKEDVTKEVMILQKVNHANLVKLTGVSLGNDGNFFLVYEYAENGSLHNWLFSKSSNVSSSMGFLNWSQRISIAIDVAMGLQYLHEHIQPCIVHMDITTSNILLDSNFKAKIANFSAARTSTNPMITKLDVFGYGMVLLELLTGKESLANNENGELAMVWSKVKGIFDQEVKREERFRKLMDPMLGEFYSLDDALSIAFWAVNCTAEKPLSRPTMGEVVLSLSLLTQHSPSTLERSWTHGLEVEVTRTLLTPVTAR

SEQ ID NO: 11 - protein, 590 aa, Abrus precatorius
MASRSHSLCLALMLFFTNIAAQSQQTNGTNFSCPADSPPSCETYVTYIAQSPNFLSLTNISNIFDTSPLSIARASNLEQEDDKLIPDQVLLIPVTCGCTGTLSYANISYEINQGDSYSFVATTLYENLTNWHAVMDLNPGLSPNMLPVGVKVIVPLFCRCPSKNQLDKGIQYLITYVWQPNDNVSLVSAKFGASLQNILTENNYGQNFTSATNLPVLIPVTHLPDLIQPHLDVKKGSIHLSVILGISLGCTVLTVVLMVLLVYVYCLKMKSLNRSASSAETADKLLSGVSGYLRKPTVYEIDVIIEATMSLSEQCKIGESVYKANIKDQVLAVKKIKEDASEELKILQKLNHGNLVKLMGVSSDNDGNCFVVYEYAENGSLDDWLFSKSSSDTSSSMASLTWCQRISIAVDVAMGLQYMHEHAYPRIVHRDITSSNILLDSNFKAKIANFSMARTFTNPMMPKIDVFAFGVVLIELLTGRKAITTKENGEVVMLWKDIWKIFDQEENREEMLRKWMDPKLESFYPIDYALSLASLAVNCTADKSLSRPTIAEIVLSLSLFTQPSPATLERSLTSGLDVEVTQIVTSIAAR

SEQ ID NO: 12 - protein, 597 aa, Cajanus cajan
MAVFSCFLPLCSQILCLALVLFSTNIASQSQQANGTNTNFSCPADSPPSCETYVTYIAQSPNFLSLTSISNLFDTSPLSIARASNLDPDNNKLIPDQILLIPVTCGNRSFANISYEINQGDTFSFLSTTSYENLTNWHVVDDLNPDLNPNMLPIGIQVVFPLFCKCPSKNQLDKGIKYLITYVWQPSDNVSLVSDKFDASPEDILSENNYGQNLTAANNLPVLIPVTRLPVLARSPSDGRKGRNHLPVIIGISLGCTLLVVVSMLLLVYVYCLKMKSLNRSASSAETADRLLSGVSGYVSKPTVYETNVIMEGTMNLSEQCKIGESVYKANIEGKVLAIKRFKEDVVEQLKILQKVNHGNLVKLMGVSSDNDGNCFVVYEYAENGSLDEWLFSKSSSETSNSMASLTWCQRISIAVDVAMGLQYMHEHAYPRIVHRDITSSNILLDSNFKAKIANFSMARTFTNPMMTKIDVFAFGVVLIELLTGRKAITTKENGEVVMLWKDIWKIFDQEEDREERLRRWMDPKLESYYPIDYALSLASLAVNCTSDKSLSRPTIAEIVLSLSLLTQPSPATLERSLTSSGLDVDATQIVTSVVAR

SEQ ID NO: 13 - protein, 597 aa, Phaseolus vulgaris
MAVFFVSLTLGAQILYVVLMFFTCIEAQSQQTNGTNFSCPSNSPPSCETYVTYISQSPNFLSLTSVSNIFDTSPLSIARASNLQHEEDKLIPGQVLLIPVTCGCTGNRSFANISYEINQGDSFYFVATTLYQNLTNWHAVMDLNPGLSPFTLPIGIQVVIPLFCKCPSKNQLDRGIKYLITHVWQPNDNVSFVSNKLGASPQDILSENNYGQNFTAASNLPVLIPVTLLPDLIQSPSDGRKHRIGLPVIIGISLGCTLLVVVSAILLVCVCCLKMKSLNRSASSAETADKLLSGVSGYVSKPTMYETGAILEATMNLSEQCKIGESVYKANIEGKVLAVKRFKEDVTEELKILQKVNHGNLVKLMGVSSDNDGNCFVVYEYAENGSLQEWLFAKSCSETLNSRTSLTWCQRISIAVDVSMGLQYMHEHAYPRIVHRDITSSNILLDSNFKAKIANFSMARTFTNPMMSKIDVFAFGVVLIELLTGRKAMTTKENGEVVMLWTDIWKIFDQEENREERLRKWMDPKLDNYYPIDYALSLASLAMNCTADKSLSRPTIAEIVLSLSLLTQPSPATLERSLTSSGLDVEATQIVTSISAR

SEQ ID NO: 14 - protein, 595 aa, Medicago truncatula; identical to SEQ ID NO: 4

SEQ ID NO: 15 - protein, 594 aa, Pisum sativum
MAIFFLPSSSHALFLALMFFVTNISAQPLQLSGTNFSCPVDSPPSCETYVTYFARSPNFLSLTNISDIFDMSPLSIAKASNIEDEDKKLVEGQVLLIPVTCGCTRNRYFANFTYTIKLGDNYFIVSTTSYQNLTNYVEMENFNPNLSPNLLPPEIKVVVPLFCKCPSKNQLSKGIKHLITYVWQANDNVTRVSSKFGASQVDMFTENNQNFTASTNVPILIPVTKLPVIDQPSSNGRKNSTQKPAFIIGISLGCAFFVVVLTLSLVYVYCLKMKRLNRSTSLAETADKLLSGVSGYVSKPTMYEMDAIMEATMNLSENCKIGESVYKANIDGRVLAVKKIKKDASEELKILQKVNHGNLVKLMGVSSDNDGNCFLVYEYAENGSLDEWLFSESSKTSNSVVSLTWSQRITVAVDVAVGLQYMHEHTYPRIIHRDITTSNILLDSNFKAKIANFSMARTSTNSMMPKIDVFAFGVVLIELLTGKKAITTMENGEVVILWKDFWKIFDLEGNREESLRKWMDPKLENFYPIDNALSLASLAVNCTADKSLSRPSIAEIVLCLSLLNQSSSEPMLERSLTSGLDVEATHVVTSIVAR

SEQ ID NO: 16 - protein, 598 aa, Glycine max
MAVFFPFLPLHSQILCLVIMLFSTNIVAQSQQDNRTNFSCPSDSPPSCETYVTYIAQSPNFLSLTNISNIFDTSPLSIARASNLEPMDDKLVKDQVLLVPVTCGCTGNRSFANISYEINQGDSFYFVATTSYENLTNWRAVMDLNPVLSPNKLPIGIQVVFPLFCKCPSKNQLDKEIKYLITYVWKPGDNVSLVSDKFGASPEDIMSENNYGQNFTAANNLPVLIPVTRLPVLARSPSDGRKGGIRLPVIIGISLGCTLLVLVLAVLLVYVYCLKMKTLNRSASSAETADKLLSGVSGYVSKPTMYETDAIMEATMNLSEQCKIGESVYKANIEGKVLAVKRFKEDVTEELKILQKVNHGNLVKLMGVSSDNDGNCFVVYEYAENGSLDEWLFSKSCSDTSNSRASLTWCQRISMAVDVAMGLQYMHEHAYPRIVHRDITSSNILLDSNFKAKIANFSMARTFTNPMMPKIDVFAFGVVLIELLTGRKAMTTKENGEVVMLWKDIWKIFDQEENREERLKKWMDPKLESYYPIDYALSLASLAVNCTADKSLSRPTIAEIVLSLSLLTQPSPATLERSLTSSGLDVEATQIVTSIAAR

SEQ ID NO: 17 - protein, 557 aa, Cicer arietinum
MSVFFLPSRSHVLFLALMLFLTNISAQSQHLSGTNFSCPVDSPPSCETYVTYIAQSPNFLSLTNISDLFDISPLSIARASNIDDEDKELIPGQVLLVPVTCGCTKHRSFANNTYTIKLGDSYILVSTTSYQNLTNYLEMEDSNPGLNPNLIPPFIKVVVPIFCRCPSKTQLNKGIKYLITYVWHANDNVSTVSSKFGASQVDILTENNYNQNFASAANLPVLIPVTRLPILAQPSSNGRKRSIQLPVIIDKLLSGVSGYVSKPTMYEMDVIMEATMNLSDQCKIGESVYKANIDGKVLAVKKTKKDASEELKILQKVNHGNLVKLMGVSSDNEGNCFLVYEYAENGSLDEWLFLESSKTSDSTVSLTWSQRIGIAVDVAVGLQYMHEHTYPRIIHRDITTSNILLDANFKAKIANFSMARTSTNPMMPKIDVFAFGVVLIELLTGKKGVTTKENGEVVIMWKDFWMIFDLEGNKEERLRKWMDPKLENFYPIDNALSLASLAVNCTADKSLSRPTIEEIVLCLNLLNQPSSEPTLERSLTFGLDVEDTQIVTSIAAR

SEQ ID NO: 18 - protein, 595 aa, Arachis hypogaea
  source 1..595: note = subsp. hypogaea
MAFFLPSLSSSIFLAFMLFSVTSIPTQSQQVNGTDFSCPVDSPSSCGTYVTYIAKSPNFLSLSNISDIFDTSPLSIARASNIKNEGDKLVPGQVLLIPVTCGCTQNQSFANITYELRQGDVYDIVSKTTYENLTNWRAVNNSNPDLNPVLLPIGVKVLFPLFCRCPSKKQLQKGIEYMITYVWQNNDNVSSVAAKFGASPVDILSENNYGGNFTAATYLPVLIPVTKLPVLTQPEASHGRKRSIQIPVIISISLGFTLVVAVIVISMVYAYLYQRKRTLNRGDLSAGTADKLLSGVSGYVSKPTVYEANEVIKATMNLSGQCKLGGTVYKAKIEGQVLAVKKVNQVVSEELNILQKVNHGNLVKLMGVSSDSDGNHFLVYEYADNGSLDGWLFSKLSLKASLTWYQRINIALDVAMGLQYLHEHTYPRIVHRDITTSNILLDSNFKAKIGNFSMVRTTTNPMISKIDVFAFGAVLIELLTGMKAMTTKADGEVVMLWKDIRKMFEVEDEKEKEECLRRWMDPKLECLYPVDYALSLATLAANCTADVSLSRPTMAEVVLGLSLLTQPSQAALERSLTSSALEAEVTHVATSITAR

SEQ ID NO: 19 - protein, 613 aa, Parasponia andersonii
MAISLYLLLFFITHISAQSPPTLATNFSCSTNSSQPSCKTYVAYFAQPPLFMDLKSISNLFGVSPSSISEASNLVSESTKLTRGQLLLIPLSCSCNGSHYFSNVTYNITMGDSYYLVSIHSFENLTNWPLVRDTNPTLNPNLLQIGTKVIFPLYCGCPSKSHSKNGIKYLITYVWQPSDDIYRVSAMFNASEVDIIIENNYQDFKAAVGYPVLIPVSRMPALSQPPYPSHSHHRSQLKHRWFLIAVISSAGALLILFLATFLVHSIGLYEKKKNLSHEESSLETTDLIQVKNFSKSDTLELQAKHDKLLPGVSVYLGKPIMYEIKMIMEATMNFNDQYKIGGSVYRAMINGSFLAVKKAKENVTEELHILQKVNHGNLVKLMGISLDRDGNCFFVYEYAENGSLDKWLNPQSSTSTSSSVGILSWSQRLNIALDVANGLQYMHEHTQPSIVHKEIRTSNILLDSRFKAKIANFSMARSAASAGMTKVDVFAFGVVLLKLLSGRKAMATRENGEIVMLWKEAKAVLEEEEKRAEKVREWIDPKLESFYPIDGALSLMTLAKACTQEKASARPSIGEVVFSLCVLTQSFSETLEPSWTCTLEGEDVVQITSPIVA

SEQ ID NO: 20 - protein, 582 aa, Parasponia andersonii
MADSYFPFQAIFLLLLLFSTLNMAASQLNNSATDFSCSDSPPSCEAYVAYFSQPPNYMNVGNISDLFGISQALIAKSSNLVSKDSPLIPQQLLLIPLTCTCTGNHYFANITYQVEPGDTYHYLSTLLFENLTNSQVMKKMNPEISPEYVLPYIDIIIPVFCRCPSKSHLKSEIQQFITYVWQPNDQVSNVSAKFNTSASEIVNENKYNNFSSAVGLPVLIPVSKLPVLARVKPPKSVRSKKQWILIGVESLGGIVLITLFATLLVYSNRLLKKRRKILEARRLEPRIIIQDKLLSGVSEYLGRPIMYDNKMVVEGTMDFSEQCRIGGSVYRGEIYGEVFAVKKTKQDITDELNLLQKVNHVNLVNLMGASYDTDGNRFLVYEYVENGSLERWLDLKPSSLAAASSSSSVQFLSWSQRIQIALDVANGLQYLHEHTQPNIAHWNIRTSTILLDSKFRAKIANFEVARPVGNPAMLKVDIFAFGIVLLALVSGKKALQTIENGEVIMLWKDLAKEVFEVEEKKEDRLRKWMDPNLQSFYPIDGALSLSSLARACIREKSSARPKMAEIVFSLSVLAQSSSPGTP

SEQ ID NO: 21 - protein, 620 aa, Malus domestica
MAISFLCSKPLCILLLLLFFTARILAQSTPSNSSTSFSCSVDAPPSCDTYVSYFARPQFMSLENISHLFGVSPLSIAKASNLVSEHIRLIAGQLLLVPISCGCSGNSYFSNITYEIKSGDSFYLVSINSFENLTDWHEVLNMNPTLDPSLLQIGQKVIFPLFCKCPSKMYTENGIKYHITYIWQPNDDISRVSSRFNVSTLDISSANNLHNDSAAVELPVVIPVSRLPALVQPKPPQGRNIFKQRWWLILIIILGGVLLVSSLLAIFAVYTRHQHKVKKALDGPGSSLESAEWFKMKEGKIDENFDLKFIQDKLLPGVSSYLGKPIMYEVKTIMEATMNLNEHCRIGGSVYRAIVDGQVLAVKNTKEDVTEELNILQKVNHANLVKLMGVSSETDGSRFLVYEYAANGSLDKWLYSKSSATSSSAELLTWNQRLSIALDIANGLQYMHEHTQRSIVHMDIRTSNILLDSKFKAKIANFSMARAAANDVTPKVDVFAFGVVLLALLSGKKGMEAKENGEAIMLWKDVRWVLEAEEEKVERLRKWMDPNLENFYPIDGALSLTALARACTQEKPSTRPSMGEVVFNLSVLTHSSSQSTLERSWTSALEAEEVLETISPIAAR

SEQ ID NO: 22 - protein, 624 aa, Fragaria vesca
  source 1..624: note = subsp. vesca
MAVSFLCSSMVCILLLFFFTSQILAQPAPQSNSTTSFSCAVDAPSSCETYVAYFVESPGYMNLENISDLFGVSVSSISQASNLASSYTGQTRLVAGQLLLVPITCGCTGNRSFANITYSIKRGDSYYVVSMYTFENLTRWPLVVEMNPALVPSLLQIGVKVIFPLFCKCPSKMYSDLGIKYLLTYVWQTNDDIFRVSAKFNISALNISGANNFDNGSPVVGQPVLIPLTKLPALSQPLPPHGKHIFKHRLMLIVIICLGVALSVASLLAIFLVHTHRLRKRQKLLNDKSLSLESAEWFRMKEGKSEEKIEMKFIQDKLLPGVSSYLGKAILYDVKTIMEATMNLNDHCGIGGSVYRAVIDGKVLAVKKTKEDVTEELNILQKVNHANLVKLMGISSEIDGVRFLVYEYAENGSLDKWLYHKTSTNSSSGAFLTWSQRLSIALDVANGLQYLHEHTQPSIVHMDIRTSNILLDSKYKAKIANFSMARTAANSVTPKVDVFSFGVILLSLLSGKKGMETTDNGEVIMLWKDVRGVLEAEEKKQEKLRAWMDPTLESFYPIDGALSLTALASACTQEKSSARPSMAEVVFNLSVLTHSSSESTLERSWNSALEVEEVLQTISPIKAR

SEQ ID NO: 23 - protein, 633 aa, Hordeum vulgare
MEPRRFLCCCLVAVLAVASRRCDAQGGAGNGTGRFACVVPAPCDTFVLYRTQSPGSLDLGAISDLFGVSRAMIAAANNLSLIDEDAALLPDQPLLVPVRCGCTGNRSFVNVTYPIHSGDTFYALALTGYENLTTPDVIQELNPQAVFNKLNVSQLVTVPLFCRCPTPAERSAGVLQQITYMWRPVDTMSRVSKLMGSDASAIAAANNVSADFTSTTMLPMLIPVARPPVLPPLRYGPSATTGDPGATKRFSGATVAASIAGSLVAVAALCVAIFGYRRYRRKKATVHSASRFASPRFCFNQNAYGIQSSSSIARMINGGDKLLTSVSQFIDKPVIFGTAEIMEATMNLDERCRIGSSYYRAKLEGEVFAVKPAKGDVSAELRMMQMVNHANLIRLAGISIGADGDYTFLVYEFAEKGSLDKWLYQKPPSSLPSSSSSVDTLSWNQRLGIALDVANGLLYMHEHTQPSMVHGDVRARNILLTADFRARISNFSVATPAMADAAATSSDVFAFGLLVLELLSGRTAMEARVGAEIGMLWRDIRAVLEAGDKRDAKLRKWMDPALGDEYYLDAALSLAGMARACTEEDAARRPKMADVVFSLSMLVQPLPVGDAFEKLWQPSSEENIRIVNEVAAR

SEQ ID NO: 24 - DNA, 1317 bp, Lotus japonicus
  source 1..1317: mol_type = unassigned DNA
ggacatgagattgaagctccaaaattagctcttttttctgatgaatacttaatgctttgttgtattcacttgattaagtgctagaaatcatctttgcatgatcatagattaaatgaatttccagttggtgtgtggagagctattttgttatgctgacatctgcaatttgcagggcatctaatgattgtcatttcttaaattattattggttgtttccgtttctttaattatctgttttaatcttgcaggtcatacaaattaaaatactagccaccacccaagacatactaaatggggtagtagagggaagggtaaggtcgataaggatgactttttattctataaaatttaggagaatttgagcttaagtggcaaggcaaacgacattactatacgaattggctttgtaccagaaacagggaacaaataatattttacaaataagctattatcatgtcagctcatttgttcaactttgatttgattaaaaattaaatgaagttgaatttgttgagctgctttattatatatgccactggatgtttccgcattctaagtgcatgtttgaaaacatttctacaattgattacgaaggaaaaattaatcatggagagaagcttatgtgcgtagcttctgtatttctgaattgattctatctgtacagtagcatttagataatgaatgatcttggttctcgctaagcatcaaaccaatctctacccttttaaaattgcaagaattataagtcatgcattgacccaaatccttctgtggttatgccccttaaaaatccggcaagacatcaagttagttggtcattagggttccaccagctagctgacaccttgtacaacaactggccgtcctaaagttgggtaagcattacaatactaaatgccattttattatattttgcgcatggttatatacctaagtaggatttgtccacagtttctttgattcggaaaggaaaaaatatttagttgacactgacagaagcagattttatatacatatattatgaaatgactcctacatgagatacacgaatctcatccccatgagttgcagtttgacagagtacacacttatcaacttgctggaatataggaaagtctaaccaatgatgtcgatccgtattgccttaattttggtaaatttagtattacatgatcattattgatatactaaaccacaggatattttattgacaatgtgaatgttccatattttcaacaatgctgattccctctgataaagaacaagttccttttctctttccctgttaactatcatttgttccccacttcacaaaca

SEQ ID NO: 25 - DNA, 1135 bp, Medicago truncatula
  source 1..1135: mol_type = unassigned DNA
cgatccttcgttgtattcacttggtcaagtgcaagaaatcatctttacacaatcatataatgaatggacctttggttgatgtgttgagagctacattcagtcctctgctgacttctgcaacttgaggggaatctaatgattgtcttattgcaattaacaattccttttaattttttgacagaaagagttggttacaaaatctcatctaacatattgttgagttcctcttttttttttttttttttttttttgataaaagggtacatctgttcgcagctctgaagattaatccctcgaatagggttggataacatgaacgacaaatttgtttcttaagagttttaacactgtcatattcttatggcagaattcgaactcgaacccactggttaagctagaagtaagaagtgacccatgccatctcattcaatcgttcttggcttgttttcggtttagatgaatatgaagtacaaaaagctacgctcaagcaaaggagagcaggtaagaagggcatcttatttttttaatatattagatgtcaagcttacgtggcgatgtaaacactgtttctatccgaattagctatgtactagaaatagggaacaaataatataaatggtgagtttgtcaaggtgttttaatatattgaaaccaatgtaactggaagtgtcaacattgaaagctgttcattgcctgtattatattataattgcatatatataatggcattagcctttgttaaacgttaaaccatatcttttgctagtttgccccttaaaaatttggtaaaggcatcaagttagatggtctttaggtaccagccagctagttgacatcgtgtatggacaccaaattgggttataataccatattattcttaccatctttattatattctgcgcatgatttcatacttgtttgggatttgccaacagtgtctaagatttgaaaaagaaaaatagtagaactaatgacagagacatcagcatatatttttaatatcaaaccaaaagatatttgatgtccaaagaagagataaatataaagttagaagtatgacaataagtcttggttgttatttgccataagaaactctcttttctcttcccctcataatttgcatttcctcacaaca

SEQ ID NO: 26 - DNA, 2496 bp, Lotus japonicus
  source 1..2496: mol_type = unassigned DNA
tcgactatataaggggttcctccatacctaataagacatgtttgtttcatgtcgatctacttactctattttatggttttctcgtgagcaatttacatataaaatacacattaaattgcacccaataactaactagttacaagattagagcacataaatcatggaatgaaacaatagcctatgacttgggtattcggtccacaacatctctagaagttcgtacaacaacgatcaaagagaaagtactgactagtgatatgagaaatttggtcaggaacaatgggctggacacattacgaacttgagcctgaatagtagcagcatatgtctgccttcgcaatgacgaagaaggttggacggagctaaagttaaagctggaagggtgtgtcgtggcatttgaaatttagggttcaactgtagctttgtttgtctaactacccttctagctttaataaaaaaacgtaaatcaattaattaaagagaaatcacgccaacctgtctcaaacccgctctaacttgttggttaaggttatgtgttgtatttcaatttgcttgttgcttcaacttgactccttacctcctttcatgttttttcttgaaaattgtgtaaactcagttttttttttttgagaatgtatgagaatgtgatacaaattaaaatacaaacatacattgaatgagcggggctaaaatgactcaaggtacatatgagttaagtttttaccaacccatctctttgatgagttgggtttaattgggttagctcacgtgttaactatttcgacaattctaactttttggaataatgccatccaaccctaggtaaaactcaacatgacttctcttaaagaaatgaagagtgaaagtctacataaatcttaaacacttaaaacactacaacttggtagatcattaaaatatgctccagaaataccataatacttgaaaataaataatttataaatgtaaaatataaatttgttgctgcacccaagtctgagtgaggaatgaataaagataaataaaatatatatatgcgttgatactaacatatgatatgatgtgatagatgtcatatataatgaacagaatgatagaaattgttgagcgcatggaagcttcttgttcaataattcaagagctgcttgtggagggctgctgcagggttgttatggatgcaattatcaaatatcttgctggaaattgatccaaaagttgactattaccttatcaatgtccggtgtccttcaagccaccattcaattagtcaataaccttttgtttttgtttttttataattttggtgccaaataattttaaccatttatttttatcatatatattatataagatggcaagtctataatcatttaaaattaaattagtctaaatttttctaatatgctgtataaatttattatatataagaataaataatattaaaaatattgtttaattgctaccagttcaatcattaattaactattgactgaaaagaaaaatgtctaacaaattttgttacattctgtttgttttgagaaaaaaaaaattgtttttcattttctgaaaatatttatttttaaatttattaaatttagaaattatgtttgtttttacgctttgttttcatcttcaccgtcaccaccatcgctaccactgtcgtcactattactatataccactaccatcgttgttgccgttgtcattgtcaacaccattgctgtcgtcgttgttattatcgccccacaaccactatcatcatcactgctatcgtcatagctaccacgaacaccaccatcacttttcattgaaaatgaaaaatgtttatttatttattttgattatattttaaaaaatatttttaatcaaccaaatttctttttaactttttttttcttatgaaaaaaaataatatgaaaagaacaaaccaaacagatccttatattcttgtttccattctttatcacccatgagagtagctgtagcgcgttaactttctaatagaggcgggaatttaccgttaaccgttgggtcatcactcagcttactgagtcatcttcttcctactgtaggcggaacccacacgccactgcctctctattattacatcaaattctattttttttacatcatcatcaagataaataaattgcattattcagaaattaattcctgaatctccctatcttaactcaggtgtctctagctattaccacttaagctattattagaagtctacgtcaaattctataataaacacatattcttattcttattattatactaatacaaaccttgatttgaagccaaaaatgataaaacaattgcagactcgttcaaacaacagacaacatgtaccattaaattccaccccaaattctcaaagcaaattctgaattctccaactagtttccattctcctagtcaactcaactcaaaacccagatgagaatgagataattccaactttaacaaacacacacaattcatcattca

SEQ ID NO: 27 - DNA, 2000 bp, Medicago truncatula
  source 1..2000: mol_type = unassigned DNA
aaataatgtgtctaaaattaacaaagatattttatggttaatttattgagcttagttaaagtcattttctaaaacgagacattgtagttgttagataaataatttcctttaattttcatcaaagtttgtaaataattatagggcaggacacatatgtccatcaattgatatatatatatcttttgtaataacgatcttatagaagaatcaatatctacaaagaaagttatcacttgtctcaaattatgctttatgcaactggtattgtcatgtagattccattttggtttatatgatattgaagtcaacgaatcacagctgcttaggcgtggttcataatttaaaattgaaactatggtgtttgttggaattatgagaggacagactgtgaagtggttgcacagctaccgacagggttgccattactgatgcaaaggattaaacaatagaacattttcaatcaacttaatctatgtttgacatcacgctcgatgtgtcagaatcacgacgacccactgtaatttttaaaaaaattacattttataatttttgtagaatcacggtgtatcacagtgattttgacatacccactgtaataccaaacatgcacttgcattctttcttccgtttgatgccaaaaatggtattgtttataatacacttaaaataattcatatccaagttgttgtataaaaaaataaacaatgtgatttcccaaaaaaaaaaaataataagtgattggcatgcaacatcagattttgttgtccaagtggtcacatttgaaatatttgagtttacatttttctcactaatatctttatattgaacttcaatctgtctagctataatcaaatggttgactaaaatggttaataagcttcaattaaattcaaattatttgaaagaacttgaatttaaatcttaacttgaacataaccccgaagtggatgatttacccaaaaaaattacctaactactctaataatcattttaacaataagagtattttactcaaagaaatttatataatcacaaaatcctagtatactcaatgattttcttaaaaaactattataaccagaaatgacaatatgagacaaatataattattttaataaaaatatctaaattagaattatttgaaaatgtaaaactatattattttttgtagttttccaaactttattaagcatcgtttaattgcacacaaaatttactccaattttttttagtctctgaaagttgctttttggtccatgttttagttttggtaattataattaagtttgttcatattaattagttctaatgtgtgtactatatagatcatactaattttgttcccaaactcatcttagttaggatctttgtatctactataagattaatccttgtgccgatataagaatatttgtcatgttatcaactatcacaatattgtattggtgagatcatataagatttgatgtcaacatcttcgtaaaggtgtcagattcgattctccccggtatcaatttaagtgagctaatttagcttcttaaaaaataaaatcaaacaacttttacataaactcagtgaaaacttggatataaagtatccttatactactctttagtcttgattagtctctgcaaagatatttatatgtactttgtattatcataagaacattcattgacattttaagttaatgaattactaacatgtcaactcttattctagccaacagttactttgttccctccacattctctttgaaatagtcaaacgtatccaatcatgcatgtctgttctgatcataacagcaaaagcatgtgtatagaaaattgatagttgaattagagtcattttccataaaaaaatattcaataagtgtgacattatttttcgtatgaattaatccattttttgctgatttgagattctttctttctttgcttcttgctttccttcatcagccattttttttgttttctctttctctctctcttcttgattca

SEQ ID NO: 28 - protein, 42 aa, Lotus japonicus
EIVLSLSFLTQQSSNPTLERSLTSSGLDVEDDAHIVTSITAR

SEQ ID NO: 29 - protein, 40 aa, Medicago truncatula
EIVLCLSLLNQPSSEPMLERSLTSGLDAEATHVVTSVIAR

SEQ ID NO: 30 - protein, 40 aa, Cajanus cajan
EIVLSLSLLTQPSPATLERSLTSSGLDVDATQIVTSVVAR

SEQ ID NO: 31 - protein, 40 aa, Phaseolus vulgaris
EIVLSLSLLTQPSPATLERSLTSSGLDVEATQIVTSISAR

SEQ ID NO: 32 - protein, 39 aa, Abrus precatorius
EIVLSLSLFTQPSPATLERSLTSGLDVEVTQIVTSIAAR

SEQ ID NO: 33 - protein, 40 aa, Lupinus angustifolius
EIVLTLSLLAQPPPASLERSLTSSGLDAEATQTIAPVAAR

SEQ ID NO: 34 - protein, 39 aa, Sphagnum fallax
EIVLCLSLLAQPSPPILERSLTSGLDVEVTQTMAPIAAR

SEQ ID NO: 35 - protein, 40 aa, Trifolium pratense
EIVLCLSLLNQPSSEPMLERSLTSGLDVEATHVVTSITAR

SEQ ID NO: 36 - protein, 40 aa, Cicer arietinum
EIVLCLNLLNQPSSEPTLERSLTFGLDVEDTQIVTSIAAR

SEQ ID NO: 37 - protein, 40 aa, Arachis hypogaea
EVVLGLSLLTQPSQAALERSLTSSALEAEVTHVATPIAAR

SEQ ID NO: 38 - protein, 40 aa, Arachis hypogaea; identical to SEQ ID NO: 37

SEQ ID NO: 39 - protein, 40 aa, Abrus precatorius
EVVLSLSLLTQHSPSTLERSWTHGLEVEVTRTLLTPVTAR

SEQ ID NO: 40 - protein, 39 aa, Cicer arietinum
EVVLSLSLLTQHSTTTLERSWNFGLDLEDTGMVTPITAR

SEQ ID NO: 41 - protein, 38 aa, Chamaecrista sp.
EVVLSLSFLTQSSPIMLERWTTGLDTEDLQLASTIAAR

SEQ ID NO: 42 - protein, 38 aa, Chamaecrista sp.
EVVLSLSFLTQSSPIMLERWTTGLDTEDLQLASPIAAR

SEQ ID NO: 43 - protein, 41 aa, Mimosa pudica
EVVLSLSFLSQTCPGVMVEQWTSPGGLDAEVPQMISPITAR

SEQ ID NO: 44 - protein, 33 aa, Cercis chinensis
EVVLSLSLPTLEGSLTSGLDAELPQMVAPIAAR

SEQ ID NO: 45 - protein, 39 aa, Chamaecrista sp.
EIVLTLSLLTQSASVDPMLDMSIDVEVEFVPPNSPVTAR

SEQ ID NO: 46 - protein, 39 aa, Medicago truncatula
EVVLSLSLLTQPSPTLLEVSWTYGLDVEVAEMVTPIIAR

SEQ ID NO: 47 - protein, 39 aa, Mimosa pudica
EIVLSLSLLIQSSSHAASLKGSCTLDFDVASLNSPVTAR

SEQ ID NO: 48 - protein, 40 aa, Lotus japonicus
EVVLSLSLLMTQHSPTTLERSWTCGLDVDVTEMQTLIAAR

SEQ ID NO: 49 - protein, 18 aa, Parasponia andersonii
EIVFSLSVLAQSSSPGTP

SEQ ID NO: 50 - protein, 40 aa, Parasponia andersonii
EVVFSLCVLTQSFSETLEPSWTCTLEGEDVVQITSPIVAR

SEQ ID NO: 51 - protein, 41 aa, Dryas drummondii
EVVFNLSVLTHSTSESTLEGSWTSTLEAEEVLQTISPITAR

SEQ ID NO: 52 - protein, 41 aa, Fragaria vesca
EVVFNLSVLTHSSSESTLERSWNSALEVEEVLQTISPIKAR

SEQ ID NO: 53 - protein, 35 aa, Discaria trinervis
EIVFNLTVLLTQSSSSETLEGSWTSTVLETEEAIH

SEQ ID NO: 54 - protein, 42 aa, Ziziphus jujuba
EIVFNLSVLLAQSSSSETFEGSWASTVETEEVVHVISPITAR

SEQ ID NO: 55 - protein, 40 aa, Juglans regia
EIVFNLSVLTQSPSETSERSWTSGAEGDQVIHIISPVTAR

SEQ ID NO: 56 - protein, 43 aa, Casuarina glauca
EVVFSISVIVQSSSEASQESWASAVHGEEEKEEIPIGTPVTAR

SEQ ID NO: 57 - protein, 26 aa, Discaria trinervis
EIVFNLYILAQSSSDGYDRSLTSGVX

SEQ ID NO: 58 - protein, 42 aa, Datisca glomerata
EIVFNLSVLAQTSPFVQEKSWVSSLEEAAEDVSHNVNPLTAR

SEQ ID NO: 59 - protein, 40 aa, Datisca glomerata
EIVFNLSILSQSTIEMQVGIWFSRVELDEVNHNISPVTAR

SEQ ID NO: 60 - protein, 39 aa, Begonia fuchsioides
EIVFNLSVLVQSANVFAQKSWVFGLEDEVGPRLNPLSPR

SEQ ID NO: 61 - protein, 39 aa, Dryas drummondii
KIVFNLSVLAQSSPKTLDRSKTSAPETEIIVEVVTQVIX

SEQ ID NO: 62 - protein, 39 aa, Dryas drummondii
EIVFNLSVLAQSSPKTLDRSKTSAPETEIIAEVVTQVIX

SEQ ID NO: 63 - protein, 41 aa, Cucumis melo
EIVFNLSVLTQSSSLISLERSWVLGLESIEISEKVDTVLAR

SEQ ID NO: 64 - protein, 40 aa, synthetic construct
EIVLSLSLLTQQSSSPTLERSWTSGLDVEVTQIVTPITAR

SEQ ID NO: 65 - protein, 15 aa, Homo sapiens
KRTLRRLLQERELVE

SEQ ID NO: 66 - protein, 15 aa, Lotus japonicus
AETADKLLSGVSGYV

SEQ ID NO: 67 - protein, 15 aa, Medicago truncatula
SETADKLLSGVSGYV

SEQ ID NO: 68 - protein, 15 aa, Cajanus cajan
AETADRLLSGVSGYV

SEQ ID NO: 69 - protein, 15 aa, Phaseolus vulgaris
AETADKLLSGVSGYV

SEQ ID NO: 70 - protein, 15 aa, Abrus precatorius
AETADKLLSGVSGYL

SEQ ID NO: 71 - protein, 15 aa, Trifolium pratense
AETADKLLSGVSGYV

SEQ ID NO: 72 - protein, 15 aa, Sphagnum fallax
VETADKLLSGVSGYV

SEQ ID NO: 73 - protein, 15 aa, Lupinus angustifolius
VETADRLLTGVSGYV

SEQ ID NO: 74 - protein, 15 aa, Cicer arietinum
SETADKLLSGVSGYV

SEQ ID NO: 75 - protein, 15 aa, Lotus japonicus
EIAGKKLISGVSNYV

SEQ ID NO: 76 - protein, 15 aa, Hordeum vulgare
INGGDKLLTSVSQFI

SEQ ID NO: 77 - protein, 15 aa, Hordeum vulgare
VGGERFLVTDISEWL

SEQ ID NO: 78 - protein, 15 aa, Hordeum vulgare
SASSSALVSSDARAA

SEQ ID NO: 79 - protein, 15 aa, Hordeum vulgare
SSSSSAFVSSDARAA

SEQ ID NO: 80 - protein, 10 aa, Homo sapiens
KRTLRRLLQE

SEQ ID NO: 81 - protein, 10 aa, Lotus japonicus
AETADKLLSG

SEQ ID NO: 82 - protein, 10 aa, Medicago truncatula
SETADKLLSG

SEQ ID NO: 83 - protein, 10 aa, Cajanus cajan
AETADRLLSG

SEQ ID NO: 84 - protein, 10 aa, Phaseolus vulgaris
AETADKLLSG

SEQ ID NO: 85 - protein, 10 aa, Abrus precatorius
AETADKLLSG

SEQ ID NO: 86 - protein, 10 aa, Trifolium pratense
AETADKLLSG

SEQ ID NO: 87 - protein, 10 aa, Sphagnum fallax
VETADKLLSG

SEQ ID NO: 88 - protein, 10 aa, Lupinus angustifolius
VETADRLLTG

SEQ ID NO: 89 - protein, 10 aa, Cicer arietinum
SETADKLLSG

SEQ ID NO: 90 - protein, 10 aa, Lotus japonicus
EIAGKKLISG

SEQ ID NO: 91 - protein, 10 aa, Hordeum vulgare
INGGDKLLTS

SEQ ID NO: 92 - protein, 10 aa, Hordeum vulgare
VGGERFLVTD

SEQ ID NO: 93 - protein, 10 aa, Hordeum vulgare
SASSSALVSS

SEQ ID NO: 94 - protein, 10 aa, Hordeum vulgare
SSSSSAFVSS

SEQ ID NO: 95 - protein, 5 aa, Homo sapiens
RELVE

SEQ ID NO: 96 - protein, 5 aa, Lotus japonicus
VSGYV

SEQ ID NO: 97 - protein, 5 aa, Medicago truncatula
VSGYV

SEQ ID NO: 98 - protein, 5 aa, Cajanus cajan
VSGYV

SEQ ID NO: 99 - protein, 5 aa, Phaseolus vulgaris
VSGYV

SEQ ID NO: 100 - protein, 5 aa, Abrus precatorius
VSGYL

SEQ ID NO: 101 - protein, 5 aa, Trifolium pratense
VSGYV

SEQ ID NO: 102 - protein, 5 aa, Sphagnum fallax
VSGYV

SEQ ID NO: 103 - protein, 5 aa, Lupinus angustifolius
VSGYV

SEQ ID NO: 104 - protein, 5 aa, Cicer arietinum
VSGYV

SEQ ID NO: 105 - protein, 5 aa, Lotus japonicus
VSNYV

SEQ ID NO: 106 - protein, 5 aa, Hordeum vulgare
VSQFI

SEQ ID NO: 107 - protein, 5 aa, Hordeum vulgare
ISEWL

SEQ ID NO: 108 - protein, 5 aa, Hordeum vulgare
DARAA

SEQ ID NO: 109 - protein, 5 aa, Hordeum vulgare
DARAA

SEQ ID NO: 110 - protein, 10 aa, synthetic construct
  VARIANT 1: note = K, A, S, V, E, or I
  VARIANT 2: note = R, E, I, N, G, A, or S
  VARIANT 3: note = T, A, G, or S
  VARIANT 4: note = L, A, G, E, or S
  VARIANT 5: note = R, D, K, or S
  VARIANT 6: note = R, K, F, or A
XXXXXXLLSG

SEQ ID NO: 111 - protein, 5 aa, synthetic construct
  VARIANT 3: note = L, G, N, Q, E, or R
VSXYV

SEQ ID NO: 112 - protein, 23 aa, Lotus japonicus
IMVAKSMEFSYQELAKATNNFSL

SEQ ID NO: 113 - protein, 23 aa, Lotus japonicus
GYVSKPNVYEIDEIMEATKDFSD

SEQ ID NO: 114 - protein, 480 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQGALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 115 - protein, 448 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQGALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLT

SEQ ID NO: 116 - protein, 504 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQGLNDIFEAQKIEWHESGGGSGGGSGALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 117 - protein, 131 aa, synthetic construct
MQVQLVETGGGLVQDGDSLRLSCAASGFTFSNYYMSWVRQAPGKGLEWVSGITSGGTYTKYADAVKDRFTISRDNAKNTLYLQMNSLKPEDTAVYYCAKGGTSVAVRGFYDYRGQGTQVTVSSLEHHHHHH

SEQ ID NO: 118 - protein, 139 aa, synthetic construct
MQVQLVETGGGLVQPGGSLRLSCAASGFTFSNYYMTWVRQAPGKGLQWVASIYGDGTYAIYADSVKGRFTISSDDSKNTVYLQMNSLEFDDTAEYYCAAAPWAVVAGTFVDGPAEDDYDYWGQGTQVTVSSLEHHHHHH

SEQ ID NO: 119 - protein, 124 aa, synthetic construct
MQVQLVESGGGSVQAGDSLRLSCTGPGRTDGPYVMGWFRQAPEKEREFVAAISRYGSGTYYADSVRGRFTISRDNVKNTVYLQMNSLKPEDTAIYYCNRVPPVLSWGQGTQVTVSSLEHHHHHH

SEQ ID NO: 120 - protein, 123 aa, synthetic construct
MQVQLVESGGGLVQAGGSLGLSCAASESTFRINAMGWYRQAPGKRRELVASISSAGETNYSPSVKGRFTISRDNAKNTVYLQMNSLKPEDTAVYYCNYLGPKGDWGQGTQVTVSSLEHHHHHH

SEQ ID NO: 121 - protein, 6 aa, synthetic construct
TLRRLL

SEQ ID NO: 122 - protein, 6 aa, synthetic construct
TADKLL

SEQ ID NO: 123 - protein, 6 aa, synthetic construct
HHHHHH

SEQ ID NO: 124 - DNA, 4171 bp, Lotus japonicus
  source 1..4171: mol_type = unassigned DNA
gcatgcatatagctctatttctttagtaatgttacacctgcacgatgtccataataatagaagtcataatacatatacagattaaaattaaataaacaatttctaatcaaatttaaaaatgtcaacttaatttcattattaaaatataacaatatgaataaccaaaaataaattaagacattcacccccccccccccgaaaagaaatttaagacaattacaattttttggtatatatattaaagacttccaattatggacataggatctcaacttagtaatcttcactttaggaaagtcttttccccacaagtcacaaccatctattaatatcaatacaaaatgaagtcaactcaataaaaagatccttttataggaaattgatgaataaaactgatatatatttcagttaaaattgttcaaacattagtgcaatggacagaagtatcctttgtgccctcatttgccaacaactggctcatcaagcaataaattaattcgccatttccaaacttttgcagttttaagtagaagatatccattcgttgaaactttcttcacaccaccaatttcctcctaaatgggttaacaaatgtgcaatgatcgaaatatatagttgaaacgatcaagatcctctcaatggtaaaagaatttgaccacgctaagttttattatctcactagctattaatttaattatcatttatcttttcaattattaaacacacaaataatcaatcctaaaatgatgaaacttgacatggtctatttttacaataacttaaccaaaaacttataagttagcaactttcaaaaacaggttttcccttgttaagaatagacaaatcaaatggagtgtgttaaatattgtgtttaaaatagtgtgttgcaagcatttctcttataaaaaatcagtataaatatgtttggaactgtttattttagtttatcttatattataaatacaaaacaagtgtttggtaaagctaatgaaaataacttaaaacatacctatggtttgtcggatgtgtcgcgggtggagctccttgccattttgtgtggcctttgtatgtgttgagatatggccatgggttattgtagaatccttcttcttttggattcttctttgttcctgtctctcattcaacgctcatgttacccatttcatccatgccaagttttttttatacatgcatctaaattttttccgccatatcttaattttgtttttattaaaattaaaaagaatatgattgaatgtcaatgtaaattttttttacacagacaatgcatatccattaaggtttgttagaattacactccaccccatttttatctaaaatctacatcccaccccattttatatagaggcaaatttagtgacgaaaaatattcttcattaataattagttattatttaaattgttaatcaataatttcaaaaaaaaattcaatataatccaataaatttaaaaatgaaaacatcacaatcctcctcattctctcaatcgcgttttacctccgtaaatttacaatgcaaattatgcaatagcacacctgcccgatttacaaaccatatttcgaacatagtgaaacatgcttgtgttttcatatttggtgataattcaattttaatcaaaataatctctttatacctccaattttcaaaattgggttgtaggccaaaaaagcaacacaaatgggtgaagaaaatagagaaacaaaattatgaaaatatgaagtggatctgaggttattagagcccaacgaggcggtggagcatcgtttttaacaaaatccaacaatatctttaggggtgaaatccaccaaccgagcgttcgctcacaacttaaaggggtgaaattcacaagaagtagtttaaaggggtgaaattcacaagaagtagttgaacaagtgactttaggaatgtgcgattcacgttctggggttcaggtcgcaacaaaactttgaggcatggtggtgccatgtggtttcacattgtcggacagtggagccgtgttaaagggagtaaaggcttggtggtggccgtttgtggtgaaaaatatgatttggagatagatgggacgtggacttaacagacagagtggatggtttttttttaaatttaattcagtaaaattatttttataaattaatgtatgatagtgtatatgcattaaatttatttaaatttttactaattagtaatttgtttttagtagtgacgaatttgtttttgtcactaaattttgcctttataaaaaatggggtggagtgtagatttttaaaaagaatggggtggagtgtcattcttgcaaatcttgaggggggtgagtgtattttactcaactcttaaaaaaattaggaattaattagttgtaaattataaaagtttatttcattgaataacataacaaattaaaggcaaaaaaatacaaaacttcattttatatgtatttcagaaaaattgcctactttcaattatgagaaactaaaattatgtttagtttaaaatgagcatagattcaaaaattaataaataatatatatagcaggatacatgcctatcaattaacatatcgtttgtccacgatgatgatcttattggaggatcaatatcttcaaattaacaaagttatcacttggctcttattggtcataatgcaataaaaaaattgcaattagtatcaaatcaaactgaaatttgcaactatatgctgctggtgttgtcgcgcagattcctttttgatttttatgggaatgaagtcaatgaagcaacagtttcacaggcgtgcttaaaaataaaaaaattggaaatttgatgtttgttaggattatgagaggacacaatgggaggatgtttcacaagctgcagacagggttgccacttcagatgcaaaggattaaataaacaaagccaaggtttgcaatcaacaagattccatcgtcgttttgcttcctttaatcgtattaatcaaaagcacaccaagtaaagcatcaatatataacatccaagaaatcacaacatgatagttgctcgtgtcgtctattaactatgatgtcaggagttcgatccccgctcatgtgaatggaagtcatttcgttgttagatgtttaccgtttaatgcaaatactcgcggtgagataataagtcattgttgtgggcgaataccctaaaataagaataaaattaaatatagcatccaagttattgcccaaatatataaacaatggtattgttgacattattaggcataaaagcagtaggtaagtgtattatatttatttaattttttaaaattttgaaattaattaataattgttaacataagtaaaccatttttagcaaaaactctacacttctattaccttaacaagtacatttttgatggtacaccttaacaattaacaagtcatatgattgacaaacatattttatatgctttacaatttattctaaaatcaaagtttatgggaagaagctcataaaagtagttcctgggtgttttttagaatagagaagttgatcatgttagaaattaagttaaaaatgagttgaaagtgatttatgtttgattatatttatgagaaaaatgaattgtctgatgtaatattgtaaaatctaacaattaattaagtaccacagaaactagaatttatagcttcaccttagaattgattttggagttaaaatcaattattaaaggagcaattattaaaggagacatccaaatacactagttaattttgacaatcaattctaacacttgcaaatgtgtaaccaaacttactatcagtaagtgaactaatgattcccaagtcaacttttgttctagctagccaaccgttactatgttccctccacaatacattctccttgaaactgtcaagtgtcaactgcacccaaacatccttgtttgtgatgaaaagatcgaaaacgtgtgcttatgaatttacatgtttacattcaccaaaaatcaaaagttacacctctatacttatcacatatgtttgagtcactttccatataaaatcccatagtctattaattatcagagtaagtgtgacattagtttcaagagaaaaataaatgatcaaaacctggtagagagtcctagaaattcaatgttctgatttctttcattcatctctgctgccattttgatttgcaca

SEQ ID NO: 125 - protein, 19 aa, Lotus japonicus
SSAETADKLLSGVSGYVSK

SEQ ID NO: 126 - protein, 19 aa, Hordeum vulgare
RMINGGDKLLTSVSQFIDK

SEQ ID NO: 127 - protein, 19 aa, synthetic construct
  VARIANT 1: note = S, E, D, P, R, or A
  VARIANT 2: note = S, L, F, M, A, R, or H
  VARIANT 3: note = A, S, V, K, I, or N
  VARIANT 4: note = E, G, F, I, N, or M
  VARIANT 5: note = T, I, G, or K
  VARIANT 6: note = A, Q, P, or G
  VARIANT 7: note = D or G
  VARIANT 8: note = K or E
  VARIANT 11: note = S, T, or P
  VARIANT 12: note = G or S
  VARIANT 15: note = G, S, E, D, or Q
  VARIANT 16: note = Y or F
  VARIANT 17: note = V, L, or I
  VARIANT 18: note = S, G, or D
  VARIANT 19: note = K, R, or Q
XXXXXXXXLLXXVSXXXXX

SEQ ID NO: 128 - protein, 34 aa, Lotus japonicus
TASSAETADKLLSGVSGYVSKPNVYEIDEIMEAT

SEQ ID NO: 129 - protein, 34 aa, Medicago truncatula
STSSSETADKLLSGVSGYVSKPTMYEIDAIMEGT

SEQ ID NO: 130 - protein, 34 aa, Pisum sativum
STSLAETADKLLSGVSGYVSKPTMYEMDAIMEAT

SEQ ID NO: 131 - protein, 34 aa, Phaseolus vulgaris
SASSAETADKLLSGVSGYVSKPTMYETGAILEAT

SEQ ID NO: 132 - protein, 34 aa, Glycine max
SASSAETADKLLSGVSGYVSKPTMYETDAIMEAT

SEQ ID NO: 133 - protein, 34 aa, Cicer arietinum
TASLSETADKLLSGVSGYVSKPTMYEMDVIMEAT

SEQ ID NO: 134 - protein, 34 aa, Lupinus angustifolius
KASFVETADRLLTGVSGYVSKPTVYEIDAIMEAT

SEQ ID NO: 135 - protein, 34 aa, Arachis hypogaea
RDSSAGTADKLLSGVSGYVSKPTVYEANEVIKAT

SEQ ID NO: 136 - protein, 34 aa, Fragaria vesca
KIEMKFIQDKLLPGVSSYLGKAILYDVKTIMEAT

SEQ ID NO: 137 - protein, 34 aa, Malus domestica
NFDLKFIQDKLLPGVSSYLGKPIMYEVKTIMEAT

SEQ ID NO: 138 - protein, 34 aa, Populus sp.
SFEAKITPDELLPGVSGYLSKPIIYEVKEIMEGT

SEQ ID NO: 139 - protein, 34 aa, Parasponia andersonii
LEPRIIIQDKLLSGVSEYLGRPIMYDNKMVVEGT

SEQ ID NO: 140 - protein, 34 aa, Datisca glomerata
KLESNIIQDKLLSGVSDYLGQPIIYEFKVIMEAT

SEQ ID NO: 141 - protein, 34 aa, Oryza sativa
IARMINGGDKLLTSVSQFIDKPIIFREEEIMEAT

SEQ ID NO: 142 - protein, 34 aa, Hordeum vulgare
IARMINGGDKLLTSVSQFIDKPVIFGTAEIMEAT

SEQ ID NO: 143 - protein, 34 aa, Zea mays
SFAHVMKGGKLLTGVSQFIDKPIIFVEEEIVEAT

SEQ ID NO: 144 - protein, 34 aa, synthetic construct
SASSAETADKLLSGVSGYVSKPXMYEXDEIMEAT

SEQ ID NO: 145 - protein, 34 aa, synthetic construct
  VARIANT 1: note = T, S, K, R, N, L, or I
  VARIANT 2: note = A, T, D, I, F, E, or L
  VARIANT 3: note = S, E, D, P, R, or A
  VARIANT 4: note = S, L, F, M, A, R, or H
  VARIANT 5: note = A, S, V, K, I, or N
  VARIANT 6: note = E, G, F, I, N, or M
  VARIANT 7: note = T, I, G, or K
  VARIANT 8: note = A, Q, P, or G
  VARIANT 9: note = D or G
  VARIANT 10: note = K or E
  VARIANT 13: note = S, T, or P
  VARIANT 14: note = G or S
  VARIANT 17: note = G, S, E, D, or Q
  VARIANT 18: note = Y or F
  VARIANT 19: note = V, L, or I
  VARIANT 20: note = S, G, or D
  VARIANT 21: note = K, R, or Q
  VARIANT 22: note = P or A
  VARIANT 23: note = N, T, I, or V
  VARIANT 24: note = V, M, L, or I
  VARIANT 25: note = Y or F
  VARIANT 26: note = E, D, R, G, or V
  VARIANT 27: note = I, M, T, A, V, N, F, or E
  VARIANT 28: note = D, G, N, K, E, or A
  VARIANT 29: note = E, A, V, T, or M
  VARIANT 30: note = I or V
  VARIANT 31: note = M, L, I, or V
  VARIANT 32: note = E or K
  VARIANT 33: note = A or G
XXXXXXXXXXLLXXVSXXXXXXXXXXXXXXXXXT

SEQ ID NO: 146 - protein, 320 aa, synthetic construct
ALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 147 - protein, 288 aa, synthetic construct
ALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLT

SEQ ID NO: 148 - protein, 320 aa, synthetic construct
ALNRTASSAETADKEESGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 149 - protein, 320 aa, synthetic construct
ALNRTASSAETADKLLSGESGEESKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 150 - protein, 345 aa, synthetic construct
GLNDIFEAQKIEWHESGGGSGGGSGALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 151 - protein, 124 aa, synthetic construct
MQVQLVESGGGSVQAGDSLRLSCTGPGRTDGPYVMGWFRQAPEKEREFVAEISRYGSGTEYADSVRGRFTISRDNVKNTVYLQMNSLKPEDTAIYYCNREPPVLSWGQGTQVTVSSLEHHHHHH

SEQ ID NO: 152 - protein, 337 aa, synthetic construct
AKLPTDISMALSTQDGNASSSAEYETSGSSGPGTASATGLTSIMVAKSMEFSYQELAKATNNFSLDNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLS

SEQ ID NO: 153 - protein, 16 aa, Lotus japonicus
GYVSKPNVYEIDEIME

SEQ ID NO: 154 - protein, 594 aa, Pisum sativum
MAIFFLPSSSHALFLALMFFVTNISAQPLQLSGTNFSCPVDSPPSCETYVTYFARSPNFLSLTNISDIFDMSPLSIAKASNIEDEDKKLVEGQVLLIPVTCGCTRNRYFANFTYTIKLGDNYFIVSTTSYQNLTNYVEMENFNPNLSPNLLPPEIKVVVPLFCKCPSKNQLSKGIKHLITYVWQANDNVTRVSSKFGASQVDMFTENNQNFTASTNVPILIPVTKLPVIDQPSSNGRKNSTQKPAFIIGISLGCAFFVVVLTLSLVYVYCLKMKRLNRSTSLAETADKLLSGVSGYVSKPTMYEMDAIMEATMNLSENCKIGESVYKANIDGRVLAVKKIKKDASEELKILQKVNHGNLVKLMGVSSDNEGNCFLVYEYAENGSLDEWLFSELSKTSNSVVSLTWSQRITVAVDVAVGLQYMHEHTYPRIIHRDITTSNILLDSNFKAKIANFSMARTSTNSMMPKIDVFAFGVVLIELLTGKKAITTMENGEVVILWKDFWKIFDLEGNREESLRKWMDPKLENFYPIDNALSLASLAVNCTADKSLSRPSIAEIVLCLSLLNQSSSEPMLERSLTSGLDVEATHVVTSIVAR

SEQ ID NO: 155 - protein, 633 aa, Malus domestica
MIMSGIVPDIQTFSGLIHHFALNADIKTVQKFFTMVRQTQSTPSNSSTSFSCSVDAPPSCDTYVSYFARPQFMSLENISHLFGVSPLSIAKASNLVSEHIRLIAGQLLLVPISCGCSGNSYFSNITYEIKSGDSFYLVSINSFENLTDWHEVLNMNPTLDPSLLQIGQKVIFPLFCKCPSKMYTENGIKYHITYIWQPNDDISRVSSRFNVSTLDISSANNLHNDSAAVGLPVVIPVSRLPALVQPKPPQGRNIFKQRWWLILIIILGGVLLVSSLLAIFAVYTRRQHKVKKALDGPGSSLESAEWFKMKEGKIDENFDLKFIQDKLLPGVSSYLGKPIMYEVKTIMEATMNLNEHCRIGGSVYRAIVDGQVLAVKNTKEDVTEELNILQKVNHANLVKLMGVSSETDGSRFLVYEYAANGSLDKWLYSKSSATSSSAELLTWNQRLSIALDIANGLQYMHEHTQRSIVHMDIRTSNILLDSKFKAKIANFSMARAAANDVTPKVDVFAFGVVLLALLSGKKGMEAKENGEAIMLWKDVRWVLEAEEEKVERLRKWMDPNLENFYPIDGALSLTALARACTQEKPSTRPSMGEVVFNLSVLTHSSSQSTLERSWTSALEAEEVLETISPIAAR

SEQ ID NO: 156 - protein, 619 aa, Populus sp.
MAISLLPFFSTQALLLLILLFSSTYVTSQAPPASNFSCPVDSPTTCSTYFTYLAQPPNFLDLGNISDLFGVSRKEIATASNLESEDTPLFPNQLLLVPKPCGCTGNRSFANITYQIQQGESFYLVSTTSFENLTRWQEVEALNPSLTPTLLHAGDKVIFPLFCKCPSNTHLESGIAYLITYVWQPSDDLTKVAAKLNASERNIVIENNYVNFTAAVYLPVLIPVSQLPVLSQQYPSPERRESRNRWIIIVAASIASALLIFLLIAFLAHKRCSYKRTKAFDRTGSCLETGDLIQTKELTKLESFEAKITPDELLPGVSGYLSKPIIYEVKEIMEGTMDLNEHYKIRGSVYRAKINGRVLAVKKTKDDVTEELKILQKVSHANLVKLMGMSSGFDREGNRFLVYEYAENGSLEKWLHPTSESSSSSAGFLTWSQRLHVALDVANGLQYMHEHTQPSIVHKDIRTTNILLDSTFRAKIANFSMARPATDSLMPKVDVFDYGVVLLQLLSGKKAMVTKENGEIVLLCKEIKAVLEIEEKREESLRKWMDPCLERFYPIDSALSLATLARLCTLEESSERPSMAEIVFNLTVLTQSPPETFERWTSGMETEDFTRLISPVTAR

SEQ ID NO: 157 - protein, 638 aa, Zea mays
MEPRHFCRALLLLLVVLLLGFRRAGAQDSTNYTVPARFACNVSSPCDTYVVYRTQSPGYLDLGSISDLFGTSQARIASANGLSSEDGVLQPGQPLLVPVRCGCTGAWSFANATYPIRQGDTFYNLARLSYENLTEYHLIHDLNPRSEPTSLQIGQEVTVPLLCRCPPARAVQSFITYVWQPGDTLSQVSKLMNATADEIAEANNVTSSSVSSASAAGLPMLIPVQQRPRLPPLLYAASAGEGRSSRSRRRALIIIGASVSGSLVALAALLVAIMAQRRYRRKKPSMRLGSPFAVNTKLSWSVNQYGHGSSNSFAHVMKGGKLLTGVSQFIDKPIIFVEEEIVEATMNLDERCKIGSTYYRAKLDGEVFAVKPAKGDVSAELRMMQMVNHANLIKLAGISIGADGDYAFLVYEFAEKASLDKWLYHNHQKPPSALLPSSSCTVPTTLSWGQRLSIALDVANGLLYMHEHTQPSMVHGDIRARNILLTADFRAKISSFSLAKPATADAAATSSDVFAFGLLLLELMSGRRAMEARIGSEIGMLWREIRAVLEAGDKREAKLRKWMDPALGSEYQMDAALSLAGMARACTDEDAARRPNMTEVVFSLSMLAQPLSVADGFEKLWQPSSEDNIRIAGSVAAR

SEQ ID NO: 158 - protein, 598 aa, Glycine max; identical to SEQ ID NO: 16

SEQ ID NO: 159 - protein, 7 aa, Lotus japonicus
TADKLLS

SEQ ID NO: 160 - protein, 7 aa, Lotus japonicus
GVSGYVS

SEQ ID NO: 161 - protein, 300 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSK

SEQ ID NO: 162 - protein, 623 aa, synthetic construct
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTAGLASGAAVGISIAGTFVLLLLAFCMYVRYQKKEEEKAKLPTDISMALSTQDGNASSSAEYETSGSSGPGTASATGLTSIMVAKSMEFSYQELAKATNNFSLDNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLSSLTEDCDDESSYESQTLINLLSVR

SEQ ID NO: 163 - protein, 792 aa, synthetic construct
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTAGLASGAAVGISIAGTFVLLLLAFCMYVRYQKKEEEKAKLPTDISMALSTQDGNASSSAEYETSGSSGPGTASATGLTSIMVAKSMEFSYQELAKATNNFSLDNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLSSLTEDCDDESSYESQTLINLLSVRGGGSGSGGGGGSGGGGSGGGGSGMQVQLVESGGGSVQAGDSLRLSCTGPGRTDGPYVMGWFRQAPEKEREFVAAISRYGSGTYYADSVRGRFTISRDNVKNTVYLQMNSLKPEDTAIYYCNRVPPVLSWGQGTQVTVSSSSMYPYDVPDYAYPYDVPDYAYPYDVPDYA

SEQ ID NO: 164 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKEESGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 165 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKFFSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 166 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGESGEESKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 167 - protein, 626 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVLVRRYRRKKATVHSASRFASPRFCFNQNAYGIQSSSSIARMINGGDKLLTSVSQFIDKPVIFGTAEIMEATMNLDERCRIGSSYYRAKLEGEVFAVKPAKGDVSAELRMMQMVNHANLIRLAGISIGADGDYTFLVYEFAEKGSLDKWLYQKPPSSLPSSSSSVDTLSWNQRLGIALDVANGLLYMHEHTQPSMVHGDVRARNILLTADFRARISNFSVATPAMADAAATSSDVFAFGLLVLELLSGRTAMEARVGAEIGMLWRDIRAVLEAGDKRDAKLRKWMDPALGDEYYLDAALSLAGMARACTEEDAARRPKMADVVFSLSMLVQPLPVGDAFEKLWQPSSEENIRIVNEVAAR

SEQ ID NO: 168 - protein, 624 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVRRYRRKKATVHSASRFASPRFCFNQNAYGIQSSSSIARMINGGDKEETSVSQFIDKPVIFGTAEIMEATMNLDERCRIGSSYYRAKLEGEVFAVKPAKGDVSAELRMMQMVNHANLIRLAGISIGADGDYTFLVYEFAEKGSLDKWLYQKPPSSLPSSSSSVDTLSWNQRLGIALDVANGLLYMHEHTQPSMVHGDVRARNILLTADFRARISNFSVATPAMADAAATSSDVFAFGLLVLELLSGRTAMEARVGAEIGMLWRDIRAVLEAGDKRDAKLRKWMDPALGDEYYLDAALSLAGMARACTEEDAARRPKMADVVFSLSMLVQPLPVGDAFEKLWQPSSEENIRIVNEVAAR

SEQ ID NO: 169 - protein, 624 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVRRYRRKKATVHSASRFASPRFCFNQNAYGIQSSSSIARMINGGDKLLTSESQEEDKPVIFGTAEIMEATMNLDERCRIGSSYYRAKLEGEVFAVKPAKGDVSAELRMMQMVNHANLIRLAGISIGADGDYTFLVYEFAEKGSLDKWLYQKPPSSLPSSSSSVDTLSWNQRLGIALDVANGLLYMHEHTQPSMVHGDVRARNILLTADFRARISNFSVATPAMADAAATSSDVFAFGLLVLELLSGRTAMEARVGAEIGMLWRDIRAVLEAGDKRDAKLRKWMDPALGDEYYLDAALSLAGMARACTEEDAARRPKMADVVFSLSMLVQPLPVGDAFEKLWQPSSEENIRIVNEVAAR

SEQ ID NO: 170 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLAGVAGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 171 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLDGVDGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 172 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVAKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 173 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVEKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 174 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRAITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 175 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRNITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 176 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNIFLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 177 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVFVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 178 - protein, 595 aa, synthetic construct
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKIDQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR